(19) United States
(12) Patent Application Publication
(10) Pub. No.: US 2026/0209828 A1
(43) Pub. Date: Jul. 23, 2026

(54) REAGENTS AND METHODS FOR NORMALIZATION

(71) Applicant: Twist Bioscience Corporation, South San Francisco, CA (US)

(72) Inventors: Siyuan CHEN, Palo Alto, CA (US); Ramsey Ibrahim ZEITOUN, Westminster, CO (US); Xuan Yu Elian LEE, San Mateo, CA (US); Steve Dojin OH, San Carlos, CA (US); Kristin Deann BUTCHER, Oakland, CA (US); Owen Kabnick SMITH, Mountain View, CA (US)

(73) Assignee: Twist Bioscience Corporation, South San Francisco, CA (US)

(21) Appl. No.: 19/157,126

(22) PCT Filed: Feb. 16, 2024
(86) PCT No.: PCT/US2024/016284

§ 371 Date: Aug. 15, 2025

Related U.S. Application Data
(60) Provisional application No. 63/485,676, filed on Feb. 17, 2023.
(60) Provisional application No. 63/511,086, filed on Jun. 29, 2023.
(60) Provisional application No. 63/550,574, filed on Feb. 6, 2024.

(51) Int. Cl. C12Q 1/6806; C40B 40/06; C40B 70/00; C40B 80/00

(52) U.S. Cl. CPC C12Q 1/6806; C40B 40/06; C40B 70/00; C40B 80/00

(57) ABSTRACT

Provided herein are compositions and methods for normalizing sequencing libraries. Further provided herein are adapter conjugates and hybrid circular adapters, composition comprising the same, and methods of generating the same. Further provided herein are methods of using the adapter conjugates, hybrid circular adapters, and compositions comprising the same to normalize genomic DNA libraries for next-generation sequencing.

### CROSS-REFERENCE TO RELATED APPLICATIONS

This application claims priority to U.S. Provisional Patent Application No. 63/485,676 filed Feb. 17, 2023, U.S. Provisional Patent Application No. 63/511,086 filed Jun. 29, 2023, and U.S. Provisional Patent Application No. 63/550,574 filed Feb. 6, 2024, each of which is hereby incorporated by reference in its entirety.

### SEQUENCE LISTING

This application contains a sequence listing submitted electronically in XML format under the file name “00415-0056-00304 Sequence Listing.xml”, which is hereby incorporated by reference in its entirety. The sequence listing was created on Feb. 16, 2024 and is 103,870 bytes in size.

### BACKGROUND

Identification of genomic variants in complex nucleic samples with high fidelity and low cost plays a central role in biotechnology, medicine, and basic biomedical research. To achieve balanced sequence loads for genomic DNA (gDNA) libraries, the number of nucleic acid molecules must be normalized across all samples. Conventional methods of normalization are inefficient as they require additional handling, reagents, time, labor, and cost to measure individual gDNA concentrations followed by a dilution step. Further, such conventional methods are prone to inaccuracies because they require manual quantitative polymerase chain reaction (qPCR) followed by numerous liquid transfer steps for dilution. In high-throughput environments, these inefficiencies and inaccuracies from normalization are compounded.

Alternative known methods of normalization require significant modifications to the library preparation workflow (e.g., use of protected adaptors followed by enzymatic processing) or are restricted to transposase-based methods, which increase errors and inaccuracies and reduce conversion efficiency. Such modifications to the library preparation workflow increase cost and decrease accuracy.

What is needed are novel reagents and methods for generating and normalizing gDNA libraries with increased efficiency while maintaining accuracy during sequencing.

### SUMMARY

Provided herein are compositions and methods for library size normalization.

Provided herein are compositions comprising: (a) a first polynucleotide adapter comprising: a first strand, wherein the first strand comprises a first terminal adapter region, a first non-complementary region, and a first yoke region; a second strand, wherein the second strand comprises a second terminal adapter region, a second non-complementary region, and a second yoke region; wherein the first yoke region and the second yoke region are complementary, wherein the first non-complementary region and the second non-complementary region are not complementary, (b) a second polynucleotide adapter comprising: a third strand, wherein the third strand comprises a third terminal adapter region, a third non-complementary region, and a third yoke region; a fourth strand, wherein the fourth strand comprises a fourth terminal adapter region, a fourth non-complementary region, and a fourth yoke region; wherein the third yoke region and the fourth yoke region are complementary, wherein the third non-complementary region and the fourth non-complementary region are not complementary, and wherein the first polynucleotide adapter and the second polynucleotide adapter are connected via linker. Further provided herein are compositions wherein Further provided herein are compositions wherein the linker is attached to the first terminal adapter region and the third terminal adapter region. Further provided herein are compositions wherein the linker is attached to the second terminal adapter region and the fourth terminal adapter region. Further provided herein are compositions wherein the linker comprises an alkene, alkyne, triazine, triazole, oxime, sulfide, amide, or cyclooctane. Further provided herein are compositions wherein the linker comprises a PEG unit. Further provided herein are compositions wherein the linker comprises 5-50 PEG units. Further provided herein are compositions wherein the linker comprises Sp18. Further provided herein are compositions wherein the linker comprises 1-10 Sp18. Further provided herein are compositions wherein one or more of the first polynucleotide adapter and the second polynucleotide adapter comprises at least one barcode. Further provided herein are compositions wherein the at least one barcode comprises one or more of a cell index, sample index, or unique molecular identifier (UMI). Further provided herein are compositions wherein one or more of the first polynucleotide adapter and the second polynucleotide adapter comprises at least one alpha thiophosphate. Further provided herein are compositions wherein the 5′ end of at least one of the strands comprises thymidine. Further provided herein are compositions wherein the linker connects the 5′ termini of one or more strands. Further provided herein are compositions wherein the linker connects the 3′ termini of one or more strands. Further provided herein are compositions wherein the linker comprises a polynucleotide. Further provided herein are compositions wherein the linker comprises at least one cleavable base. Further provided herein are compositions wherein the linker comprises uracil. Further provided herein are compositions wherein the linker comprises at least one restriction endonuclease site. Further provided herein are compositions wherein the linker comprises a duplex region. Further provided herein are compositions wherein the linker comprises at least one splint polynucleotide. Further provided herein are compositions wherein the at least one splint polynucleotide is at least partially complementary to a portion of the first polynucleotide adapter or the second polynucleotide adapter. Further provided herein are compositions wherein the linker comprises at least two splint polynucleotides, wherein the at least two splint polynucleotides are at least partially overlapped with each other. Further provided herein are compositions wherein the linker comprises at least two splint polynucleotides, wherein the at least two splint polynucleotides are at least partially overlapped with a portion of the first polynucleotide adapter or the second polynucleotide adapter. Further provided herein are compositions wherein the first polynucleotide adapter and the second polynucleotide adapter are covalently attached to the linker. Further provided herein are compositions wherein the first polynucleotide adapter and the second polynucleotide adapter are non-covalently attached to the linker. Further provided herein are compositions wherein one or more of the first polynucleotide adapter and the second polynucleotide adapter comprises at least one barcode.

Provided herein are adapter-ligated sample polynucleotides comprising a composition provided herein, wherein the composition further comprises at least one sample polynucleotide. Further provided herein are adapter-ligated sample polynucleotide wherein the at least one sample polynucleotide is attached to the 3′ end of the first strand and the 5′ end of the second strand. Further provided herein are adapter-ligated sample polynucleotide wherein the at least one sample polynucleotide is attached to the 3′ end of the third strand and the 5′ end of the fourth strand. Further provided herein are adapter-ligated sample polynucleotide wherein the at least one sample polynucleotide is attached to the 3′ end of the first strand and the 3′ end of the second strand. Further provided herein are adapter-ligated sample polynucleotide wherein the at least one sample polynucleotide is attached to the 5′ end of the third strand and the 5′ end of the fourth strand. Further provided herein are adapter-ligated sample polynucleotide wherein the sample polynucleotides comprise genomic DNA. Further provided herein are adapter-ligated sample polynucleotide wherein the sample polynucleotides comprise cDNA. Further provided herein are adapter-ligated sample polynucleotide wherein the sample polynucleotides comprise cDNA.

Provided herein are libraries of polynucleotides comprising a plurality of adapter-ligated sample polynucleotides disclosed herein. Provided herein are a plurality of sequencing libraries described herein. Further provided herein are libraries wherein each library is obtained from a different sample. Further provided herein are libraries wherein no more than 70% of the sample polynucleotides are present within one standard deviation of the mean sample polynucleotide amount. Further provided herein are libraries wherein no more than 50% of the sample polynucleotides are present within one standard deviation of the mean sample polynucleotide amount. Further provided herein are libraries wherein no more than 25% of the sample polynucleotides are present within one standard deviation of the mean sample polynucleotide amount.

Provided herein are methods of library preparation comprising: providing a plurality of sample polynucleotides; and ligating at least one composition provided herein to at least one sample polynucleotide. Further provided herein are methods wherein the sample polynucleotides comprise genomic DNA. Further provided herein are methods wherein the sample polynucleotides comprise cDNA. Further provided herein are methods wherein the molar ratio of the at least one composition to plurality of sample polynucleotides is no more than 1:5. Further provided herein are methods wherein the molar ratio of the at least one composition to plurality of sample polynucleotides is no more than 1:2. Further provided herein are methods wherein the molar ratio of the at least one composition to plurality of sample polynucleotides is no more than 1:1. Further provided herein are methods wherein ligating occurs with an efficiency of at least 25%. Further provided herein are methods wherein ligating occurs with an efficiency of at least 50%. Further provided herein are methods wherein ligating occurs with an efficiency of at least 75%. Further provided herein are methods wherein the method further comprises cleaving the linker. Further provided herein are methods wherein cleaving the linker comprises contacting the conjugate with an enzyme. Further provided herein are methods wherein cleaving the enzyme comprises USER or a site-specific restriction endonuclease.

Provided herein are conjugate comprising: a first strand, wherein the first strand comprises a first terminal adapter region, a first non-complementary region, and a first yoke region; a second strand, wherein the second strand comprises a second terminal adapter region, a second non-complementary region, and a second yoke region; wherein the first strand and the second strand are connected via linker. Further provided herein are conjugates wherein the linker is attached to the 5′ end of the first strand and the 5′ end of the second strand. Further provided herein are conjugates wherein the linker is attached to the 3′ end of the first strand and the 3′ end of the second strand.

Provided herein are methods of generating a composition or conjugate provided herein comprising: contacting the first strand with the second strand, wherein the first strand comprises a first reactive group and the second strand comprises a second reactive group, wherein reaction of the first reactive group and the second reactive group generates the conjugate. Further provided herein are methods wherein the first strand further comprises a first linker. Further provided herein are methods wherein the first strand further comprises a second linker. Further provided herein are methods wherein the first linker comprises the first reactive group. Further provided herein are methods wherein the second linker comprises the second reactive group. Further provided herein are methods wherein the first reactive group or the second reactive group.

Provided herein are methods of generating a composition or conjugate provided herein comprising: (a) coupling a nucleotide monomer to a growing chain on a surface; (b) repeating step (a) to generate a first strand; (c) coupling a linker comprising a reactive group to a terminus of the first strand; and (d) repeating step (a) to generate a third strand. Further provided herein are methods wherein the method further comprises cleaving the composition from the solid support. Further provided herein are methods wherein the method comprises hybridizing the second strand to the first strand. Further provided herein are methods wherein the method comprises hybridizing the fourth strand to the third strand.

### BRIEF DESCRIPTION OF THE DRAWINGS

FIG. 1A is an illustration depicting a fully assembled adapter comprising a 5′-5′ sequence annealed to a reverse-complement sequence so as to create two adapters covalently linked by a linker comprising a hexaethylene glycol spacer 18 (Sp18) group, according to aspects of the present disclosure;

FIG. 1B is an illustration depicting an adapter that may be conjugated to another adapter, or fragment thereof, where each adapter comprises a primer binding region (striped), a non-complementary region (white solid), and a yoke region (dotted), according to aspects of the present disclosure;

FIG. 2A is an illustration depicting a 5′ linked polynucleotide that may be subsequently assembled with reverse-complement sequences to create an adapter, according to aspects of the present disclosure;

FIG. 2B is an illustration depicting a 3′ linked polynucleotide that may be subsequently assembled with reverse-complement sequences to create an adapter, according to aspects of the present disclosure;

FIG. 2C is an illustration depicting an exemplary method for synthesis of hybrid circular adapters using “click” chemistry, according to aspects of the present disclosure;

FIG. 3A is a line graph depicting a simulated ligation of gDNA as a function of adapter input for standard adapters, where the solid line demonstrates relative gDNA conversion, as shown by amount of adapter used (gDNA amount out) as amount of adapter input (gDNA amount in) increases, and the dotted line represents the point at which adapter and gDNA amounts are equal, according to aspects of the present disclosure;

FIG. 3B is a line graph depicting simulated ligation of gDNA as a function of adapter input for hybrid circular adapters as provided herein, where the solid line demonstrates relative gDNA conversion, as shown by amount of adapter used (gDNA amount out) as amount of adapter input (gDNA amount in) increases, and the dotted line represents the point at which adapter and gDNA amounts are equal, according to aspects of the present disclosure;

FIG. 4A is a bar graph depicting read representation as a function of the ratio of gDNA to adapter; as shown in the graph, read representation remained between 0.6-1.3 even as the mass input of gDNA is increased relative to the input of adapter, according to aspects of the present disclosure;

FIG. 4B is a scatter plot graph depicting read representation at seven different gDNA mass amounts between 1-100 ng; as shown in the graph, read representation remained between 0.6-1.3 across the various amounts, according to aspects of the present disclosure;

FIG. 5 is a panel of two bioanalyzer readouts assessing cleavage of cleavable bases from exemplary adapter conjugates (e.g., hybrid circular adapters), according to aspects of the present disclosure;

FIG. 6A is an illustration depicting an exemplary adapter having a duplex linkage, according to aspects of the present disclosure;

FIG. 6B is an illustration depicting an exemplary adapter having an overhang linkage, according to aspects of the present disclosure;

FIG. 7A is an illustration depicting barcoded adapter pairings that are matched by adapter (A or B), according to aspects of the present disclosure;

FIG. 7B is an illustration depicting barcoded adapter pairings that are mismatched (cross-talked) with both adapters (A and B), according to aspects of the present disclosure;

FIG. 7C is a bar graph depicting relative amounts of reads with pairs of various barcoded adapters, according to aspects of the present disclosure;

FIG. 8A is a panel of six illustrations depicting various exemplary adapters, according to aspects of the present disclosure;

FIG. 8B is a bar graph depicting percentage of linear conflicting barcodes for adapters modified to have varying lengths of overhangs and to have varying numbers of spacers, according to aspects of the present disclosure;

FIG. 8C is a bar graph depicting passing-filter normalized reads across varying mass (ng) inputs of gDNA samples, according to aspects of the present disclosure;

FIG. 9A is an illustration depicting an exemplary inline barcoded adapter, according to aspects of the present disclosure;

FIG. 9B is a bar graph depicting passing-filter normalized reads across sixty barcoded adapters, each having a unique inline barcode, according to aspects of the present disclosure;

FIG. 9C is a bar graph depicting conversion rates (as measured by normalized matches) during ligation across twelve unique barcoded adapters, according to aspects of the present disclosure;

FIG. 10A is a histogram depicting 1,024 possible barcoded adapters, the left cluster containing 992 mismatched (cross-talked) barcodes and the right cluster containing 32 matched barcodes, according to aspects of the present disclosure;

FIG. 10B is a bar graph depicting amount of cross-talking after pooling ligation reaction at an initial test of three groups of barcoded adapters which would later undergo no additional steps (none), heat denaturation step (heat quench), or bead purification step (beads); as shown in the graph, prior to any additional steps, all three groups showed a relatively high amount of cross-talking;

FIG. 10C is a bar graph depicting amount of cross-talking in the groups of FIG. 10B, after the additional steps of heat denaturation or bead purification; as shown in the graph, amount of cross-talk was drastically reduced after heat denaturation and after bead purification, according to aspects of the present disclosure;

FIG. 11A is a bar graph depicting passing-filter normalized reads across human gDNA samples of six different mass inputs sequenced after ligation with twelve exemplary adapters, according to aspects of the present disclosure;

FIG. 11B is a bar graph depicting passing-filter normalized reads across maize gDNA samples of six different mass inputs sequenced after ligation with twelve exemplary adapters, according to aspects of the present disclosure;

FIG. 12 is a bar graph depicting fraction chimeras across human and maize gDNA samples of six different mass inputs sequenced after ligation with twelve exemplary adapters, according to aspects of the present disclosure;

FIG. 13 is a bar graph depicting mean target coverage after target enrichment across human and maize gDNA samples of six different mass inputs sequenced after ligation with twelve exemplary adapters, according to aspects of the present disclosure.

FIG. 14 is a bar graph depicting uniformity (fold 80 base penalty) after target enrichment across human and maize gDNA samples of six different mass inputs sequenced after ligation with twelve exemplary adapters, according to aspects of the present disclosure;

FIG. 15 is a genome coverage plot depicting genome coverage after target enrichment across human gDNA samples of six different mass inputs sequenced after ligation with twelve exemplary adapters, according to aspects of the present disclosure; and

FIG. 16 is a genome coverage plot depicting genome coverage after target enrichment across maize gDNA samples of six different mass inputs sequenced after ligation with twelve exemplary adapters, according to aspects of the present disclosure.

### DETAILED DESCRIPTION

Provided herein are adapter conjugates and compositions comprising the same. Further provided herein are compositions and methods for generating synthetic polynucleotide libraries. Further provided herein are compositions and methods for polynucleotide sample normalization.

### Definitions

The terminology used herein is for the purpose of describing particular embodiments only and is not intended to be limiting of any embodiment.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is intended to mean the stated number and numbers +/−10% thereof, or 10% below the lower listed limit and 10% above the higher listed limit for the values listed for a range.

As used herein, the terms “polynucleotide,” “oligonucleotide,” “oligo,” “oligonucleic acid,” and “nucleic acid molecule” are used interchangeably. The terms refer to nucleic acid molecules (e.g., DNA, RNA) that may be single stranded, double stranded, or triple stranded. Double stranded and triple stranded nucleic acid molecules need not be coextensive, e.g., a double stranded nucleic acid molecule need not be double stranded along the entire length of both strands. Unless stated otherwise, sequences of nucleic acid molecules, when provided, are listed in the 5′ to 3′ direction. The length of a nucleic acid molecule, when provided, is described as the number of bases and may be abbreviated, e.g., nt (nucleotides), bp (base pairs), kb (kilobases), Mb (megabases), or Gb (gigabases). Methods described herein provide for the generation of isolated nucleic acid molecules. Methods described herein additionally provide for the generation of isolated and purified nucleic acid molecules. As described herein, a polynucleotide may encode for genes or gene fragments from an organism. Exemplary organisms include, without limitation, prokaryotes (e.g., bacteria) and eukaryotes (e.g., mice, rabbits, humans, and non-human primates).

As used herein, the terms “synthetic” or “synthesized” refer to polynucleotides that are chemically synthesized and/or synthesized de novo.

As used herein, the term “library” refers to libraries of synthetic polynucleotides as described herein, which may comprise a plurality of polynucleotides collectively encoding for one or more genes or gene fragments. In some instances, the polynucleotide library comprises coding or non-coding sequences. In some instances, the polynucleotide library encodes for a plurality of circular DNA (cDNA) sequences. Reference gene sequences from which the cDNA sequences are based may contain introns, whereas cDNA sequences exclude introns. In some instances, the polynucleotide library comprises one or more polynucleotides, each of the one or more polynucleotides encoding sequences for multiple exons. Each polynucleotide within a library described herein may encode a different (e.g., non-identical) sequence. In some instances, each polynucleotide within a library described herein comprises at least one portion that is complementary to the sequence of another polynucleotide within the library. A polynucleotide library described herein may comprise at least 10, 20, 50, 100, 200, 500, 1,000, 2,000, 5,000, 10,000, 20,000, 30,000, 50,000, 100,000, 200,000, 500,000, 1,000,000, or more than 1,000,000 polynucleotides. A polynucleotide library described herein may have no more than 10, 20, 50, 100, 200, 500, 1,000, 2,000, 5,000, 10,000, 20,000, 30,000, 50,000, 100,000, 200,000, 500,000, or no more than 1,000,000 polynucleotides. A polynucleotide library described herein may comprise 10 to 500, 20 to 1000, 50 to 2000, 100 to 5000, 500 to 10,000, 1,000 to 5,000, 10,000 to 50,000, 100,000 to 500,000, or 50,000 to 1,000,000 polynucleotides. A polynucleotide library described herein may comprise about 370,000, 400,000, 500,000, or more than 500,000 different polynucleotides.

As used herein, the terms “preselected sequence,” “predefined sequence,” and “predetermined sequence” are used interchangeably. The terms refer to the sequence of a polymer which is known and chosen before synthesis or assembly of the polymer. In particular, various aspects of the invention are described herein with regard to the preparation of nucleic acid molecules, the sequences of polynucleotides known and chosen before synthesis or assembly of the nucleic acid molecules.

As used herein, the terms “electrophile group,” electrophile,” and the like refer to an atom, or a group of atoms, that can accept an electron pair to form a covalent bond. The term “electrophilic group” includes, but is not limited to, halide containing compounds, carbonyl contain compounds, and epoxide containing compounds. Common electrophiles include halides (e.g., thiophosgene, glycerin dichlorohydrin, phthaloyl chloride, succinyl chloride, chloroacetyl chloride, chlorosuccinyl chloride, etc.), ketones (e.g., chloroacetone, bromoacetone, etc.), aldehydes (e.g., glyoxal, etc.), isocyanates (e.g., hexamethylene diisocyanate, tolylene diisocyanate, meta-xylylene diisocyanate, cyclohexylmethane-4,4-diisocyanate, etc.), and derivatives of these compounds.

As used herein, the terms “nucleophilic group,” “nucleophile,” and the like refer to an atom, or a group of atoms, that have an electron pair capable of forming a covalent bond. Groups of this type may be ionizable groups that react as anionic groups. The term “nucleophilic group” includes, but is not limited to, hydroxyl, primary amines, secondary amines, tertiary amines, and thiols.

### Polynucleotide Adapter Conjugates

Conjugation can occur by joining two polynucleotide molecules (e.g., two adapters), such as through hybridization, chemical coupling of different groups, or enzymatically. In some instances, adapters are conjugated using solid phase synthesis. In some instances, adapters are conjugated via a nucleotide coupling process. In some instances, a linker is added during one or more steps of the nucleotide coupling process to generate the conjugate. In some instances, reverse amidites (e.g., phosphoramidite on the 3′ position) are employed. For example, a series of nucleotide monomers are added in the 3′ to 5′ direction to form a first polynucleotide, a linker is added, and then a series of reverse amidites are added to form a 5′ to 3′ second polynucleotide.

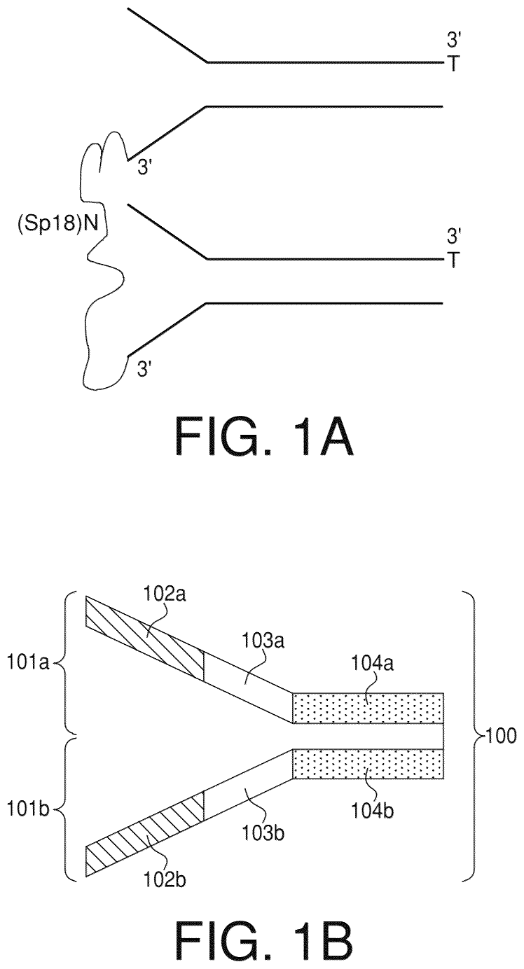

In some instances, two universal adapters are conjugated. As depicted in FIG. 1B, in some instances, the universal adapters disclosed herein may comprise a universal polynucleotide adapter 100 comprising a first strand 101a and a second strand 101b. In some instances, the first strand 101a comprises a first primer binding region 102a, a first non-complementary region 103a, and a first yoke region 104a. In some instances, the second strand 101b comprises a second primer binding region 102b, a second non-complementary region 103b, and a second yoke region 104b. In some instances, a primer (e.g., 102a/102b) binding region allows for PCR amplification of the universal polynucleotide adapter 100. In some instances, a primer (e.g., 102a/102b) binding region allows for PCR amplification of the polynucleotide adapter 100 and concurrent addition of one or more barcodes to the polynucleotide adapter. In some instances, the first yoke region 104a is complementary to the second yoke region 104b. In some instances, the first non-complementary region 103a is not complementary to the second non-complementary region 103b. In some instances, the universal polynucleotide adapter 100 is a Y-shaped or forked adapter. In some instances, one or more yoke regions comprise nucleobase analogues that raise the Tm between a first yoke region and a second yoke region. Primer binding regions as described herein may be in the form of a terminal adapter region of a polynucleotide. In some instances, a universal adapter comprises one index sequence. In some instances, a universal adapter comprises one unique molecular identifier (UMI).

Conjugation (e.g., of two adapters) can occur by reacting a nucleophilic reactive group of one adapter to an electrophilic reactive group of another adapter. In some instances, a first adapter and a second adapter are linked by reacting a nucleophilic reactive moiety on the first adapter with an electrophilic reactive moiety on the second adapter. In some instances, a first adapter comprises a linker, wherein the linker comprise a reactive group and a second adapter comprises a complementary reactive group. Non-limiting examples of nucleophilic reactive groups include amino, thiol, and hydroxyl. Non-limiting examples of electrophilic reactive groups include carboxyl, acyl chloride, anhydride, ester, succinimide ester, alkyl halide, sulfonate ester, maleimido, haloacetyl, and isocyanate. In some instances, an adapter comprises a reactive group. In some instances, an adapter comprises a linker. In some instances, a linker comprises a reactive group. In some instances, a linker connects a 5′ terminus of a first polynucleotide adapter (or fragment thereof) with a 5′ terminus of a second polynucleotide adapter (or fragment thereof). In some instances, a linker connects a 3′ terminus of a first polynucleotide adapter (or fragment thereof) with a 3′ terminus of a second polynucleotide adapter (or fragment thereof). In some instances, a linker comprises a polynucleotide. In some instances, a first polynucleotide adapter and a second polynucleotide adapter are covalently attached to the linker. In some instances, a first polynucleotide adapter and a second polynucleotide adapter are non-covalently attached to the linker.

Provided herein are conjugates comprising one or more polynucleotide strands. In some instances, a conjugate comprises a first strand. In some instances, a conjugate comprises a second strand. In some instances, a conjugate comprises a first strand and a second strand. In some instances, the first strand comprises one or more of a first terminal adapter region, a first non-complementary region, and a first yoke region. In some instances, the second strand comprises one or more of a second terminal adapter region, a second non-complementary region, and a second yoke region. In some instances, the first strand and the second strand are connected via a linker. In some instances, the linker is attached to the 5′ end of the first strand and the 5′ end of the second strand. In some instances, the linker is attached to the 3′ end of the first strand and the 3′ end of the second strand.

Provided herein are compositions comprising linked adapters (adapter conjugates). In some instances, the composition comprises a first polynucleotide adapter and a second polynucleotide adapter. In some instances, the first polynucleotide adapter comprises one or more of a first strand. In some instances, the first polynucleotide adapter comprises one or more of a second strand. In some instances, the first polynucleotide adapter comprises a first strand and a second strand. In some instances the first strand comprises one or more of a first non-complementary region, and a first yoke region. In some instances the second strand comprises one or more of a second non-complementary region, and a second yoke region. In some instances, the first yoke region and the second yoke region are complementary, wherein the first non-complementary region and the second non-complementary region are not complementary. In some instances, the second polynucleotide adapter comprises one or more of a third strand. In some instances, the second polynucleotide adapter comprises one or more of a fourth strand. In some instances, the second polynucleotide adapter comprises a third strand and a fourth strand. In some instances, the third strand comprises one or more of a third non-complementary region, and a third yoke region. In some instances, the fourth strand comprises one or more of a fourth non-complementary region, and a fourth yoke region. In some instances, the third yoke region and the fourth yoke region are complementary, wherein the third non-complementary region and the fourth non-complementary region are not complementary. In some instances, the first polynucleotide adapter and the second polynucleotide adapter are connected via a linker. In some instances, the linker is attached to the first terminal adapter region and the third terminal adapter region. In some instances, the linker is attached to the second terminal adapter region and the fourth terminal adapter region. In some instances, an adapter comprises at least one barcode. In some instances the barcode comprises one or more of a cell index, sample index, or unique molecular identifier (UMI). In some instances, an adapter comprises at least one exonuclease resistant base. In some instances, the at least one exonuclease resistant base comprises an alpha thiophosphate. In some instances, the 5′ of at least one strand comprises thymidine. In some instances, compositions comprise two or more adapter conjugates.

In some embodiments, conjugation can be carried out through organosilanes, for example, aminosilane treated with glutaraldehyde; carbonyldiimidazole (CDI) activation of silanol groups; or utilization of dendrimers. A variety of dendrimers are known in the art and include poly (amidoamine) (PAMAM) dendrimers, which are synthesized by the divergent method starting from ammonia or ethylenediamine initiator core reagents; a sub-class of PAMAM dendrimers based on a tris-aminoethylene-imine core; radially layered poly(amidoamine-organosilicon) dendrimers (PAMAMOS), which are inverted unimolecular micelles that consist of hydrophilic, nucleophilic polyamidoamine (PAMAM) interiors and hydrophobic organosilicon (OS) exteriors; Poly (Propylene Imine) (PPI) dendrimers, which are generally poly-alkyl amines having primary amines as end groups, while the dendrimer interior consists of numerous of tertiary tris-propylene amines; Poly (Propylene Amine) (POPAM) dendrimers; Diaminobutane (DAB) dendrimers; amphiphilic dendrimers; micellar dendrimers which are unimolecular micelles of water soluble hyper branched polyphenylenes; polylysine dendrimers; and dendrimers based on poly-benzyl ether hyper branched skeleton.

In some embodiments, conjugation can be carried out through olefin metathesis. In some embodiments, a first adapter, a second adapter, and/or a linker (L) comprise an alkene or alkyne moiety that is capable of undergoing metathesis. In some embodiments a suitable catalyst (e.g., copper, ruthenium) is used to accelerate the metathesis reaction. Suitable methods of performing olefin metathesis reactions are described in the art. See, e.g., Schafmeister et al., J Am Chem Soc, 2000, Vol. 122, Pages 5891-5892; Walensky et al., Science, 2004, Vol. 305, Pages 1466-1470; and Blackwell et al., Angew Chem Int Ed., 1998, Vol. 37, Pages 3281-3284.

In some embodiments, conjugation can be carried out using click chemistry. A “click reaction” is wide in scope and easy to perform, uses only readily available reagents, and is insensitive to oxygen and water. In some embodiments, the click reaction is a cycloaddition reaction between an alkynyl group and an azido group to form a triazolyl group. In some embodiments, the click reaction uses a copper or ruthenium catalyst. Suitable methods of performing click reactions are described in the art. See, e.g., Kolb et al., Drug Discovery Today, 2003, Vol. 8, Pages 1128-1137; Kolb et al., Angew Chem Int Ed., 2001, Vol. 40, Pages 2004-2021; Rostovtsev et al., Angew. Chem. Int. Ed. 41:2596 (2002); Tornoe et al., J Org Chem., 2002, Vol. 67, Pages 3057-3064; Manetsch et al., J Am Chem Soc., 2004, Vol. 126, Pages 12809-12818; Lewis et al., Angew. Chem. Int. Ed. 41:1053 (2002); Speers et al., J Am Chem Soc., 2003, Vol. 125, Pages 4686-4687; Chan et al., Organic Letters, 2004, Vol. 6, Pages 2853-2855; Zhang et al., J Am Chem Soc., 2005, Vol. 127, Pages 15998-15999; and Waser et al., J Am Chem Soc., 2005, Vol. 127, Pages 8294-8295. In some instances, click reactions are performed “copper-free” using strained olefins, such as trans-cyclooctene. Indirect conjugation via high affinity specific binding partners (e.g., streptavidin/biotin, avidin/biotin, or lectin/carbohydrate) is also contemplated.

Adapters may be connected via a linker L, e.g., in the formula adapter-L-adapter. In some embodiments, L is a linking group. In some embodiments, L is a bifunctional linker and comprises only two reactive groups before conjugation to adapters. In embodiments where both adapters have electrophilic reactive groups, L comprises two of the same nucleophilic group or two different nucleophilic groups (e.g., amine, hydroxyl, or thiol) before conjugation to adapters. In embodiments where both a first adapter and a second adapter have nucleophilic reactive groups, L comprises two of the same electrophilic group or two different electrophilic groups (e.g., carboxyl group, activated form of a carboxyl group, or compound with a leaving group) before conjugation to an adapter. In embodiments where a first adapter comprises a nucleophilic reactive group and a second adapter comprises an electrophilic reactive group, L comprises one nucleophilic reactive group and one electrophilic group before conjugation to the adapters.

The linker can be any molecule with at least one reactive group (before conjugation to adapters) capable of reacting with each of the adapters. In some embodiments, the linker has only two reactive groups and is bifunctional. Before conjugation to the adapters, the linker can be represented by the formula A-L-B, wherein A and B are independently nucleophilic or electrophilic reactive groups. In some embodiments, A and B are both nucleophilic reactive groups. In some embodiments, A and B are both electrophilic reactive groups. In some embodiments, A is a nucleophilic reactive group and B is an electrophilic reactive group. In some embodiments, A is an electrophilic reactive group and B is a nucleophilic reactive group.

In some embodiments, A and B may include alkene and/or alkyne functional groups that are suitable for olefin metathesis reactions. In some embodiments, A and B include moieties that are suitable for click chemistry (e.g., an alkene moiety, an alkyne moiety, a nitrile moiety, or an azide moiety). Other non-limiting examples of reactive groups (A and B) include pyridyldithiol, aryl azide, diazirine, carbodiimide, and hydrazide.

In some embodiments, the linker is hydrophobic. Hydrophobic linkers or linking groups are known in the art. See, e.g., Bioconjugate Techniques, G. T. Hermanson (Academic Press, San Diego, CA, 1996), which is hereby incorporated by reference in its entirety. Suitable hydrophobic linking groups known in the art include, for example, 8-hydroxy octanoic acid and 8-mercaptooctanoic acid. Before conjugation, the hydrophobic linker comprises at least two reactive groups (A and B), as described herein and as shown in the formula A-(hydrophobic linking group)-B.

In some embodiments, the hydrophobic linker comprises either a maleimido or an iodoacetyl group, and either a carboxylic acid or an activated carboxylic acid (e.g., NHS ester), as the reactive groups. In these embodiments, the maleimido or iodoacetyl group can be coupled to a thiol moiety on a first adapter and the carboxylic acid or activated carboxylic acid can be coupled to an amine on a second adapter with or without the use of a coupling agent. Any coupling agent known to one skilled in the art can be used to couple the carboxylic acid with the free amine such as, for example, DCC, DIC, HATU, HBTU, TBTU, and other activating agents described herein. In some embodiments, the hydrophilic linking group comprises an aliphatic chain of 2-100 methylene groups, wherein A and B are carboxyl groups or derivatives thereof (e.g., succinic acid). In some embodiments, the L is iodoacetic acid.

In some instances, before conjugation to adapters, a hydrophilic linking group comprises at least two reactive groups (A and B), as described herein and as shown below: A-(hydrophilic linking group)-B. In specific embodiments, the linking group comprises polyethylene glycol (PEG). In some instances, a PEG “unit” comprises —(OCH₂CH₂)—. The PEG in certain embodiments has a molecular weight of about 100 Daltons to about 10,000 Daltons, e.g. about 500 Daltons to about 5000 Daltons. The PEG in some embodiments has a molecular weight of about 10,000 Daltons to about 40,000 Daltons. In some instances, a PEG-containing linker comprises an Sp3, Sp9, Sp12, or Sp18 (hexaethylene glycol spacer 18; containing 18 PEG units) group. In some instances, a PEG-containing linker comprises about 1, 2, 3, 4, 5, 6, 8, 10, 12, 14, 16, 18, 20, 22, 24, 30, 40, or about 50 PEG units. In some instances, a PEG-containing linker comprises 1-1000, 1-500, 1-250, 1-200, 1-150, 1-100, 1-80, 1-75, 1-60, 1-50, 1-40, 1-30, 1-25, 1-20, 5-1000, 5-500, 5-250, 5-200, 5-80, 5-75, 5-60, 5-50, 5-40, 5-30, 5-25, 5-20, 10-1000, 10-500, 10-250, 10-200, 10-150, 10-100, 10-80, 10-75, 10-60, 10-50, 10-40, 10-30, 10-25, 10-20, 12-50, 12-40, 12-30, 12-20, 15-50, 15-30, 15-25, 15-20, 17-19, 17-25, 17-35, 18-50, 18-30, 18-25, 20-30, 20-50, 30-50, or 50-100 units. In some instances, a PEG-containing linker comprises no more than 1, 2, 3, 4, 5, 6, 8, 10, 12, 14, 16, 18, 20, 22, 24, 30, 40, or no more than 50 PEG units. In some instances, a PEG-containing linker comprises at least 1, 2, 3, 4, 5, 6, 8, 10, 12, 14, 16, 18, 20, 22, 24, 30, 40, or at least 50 PEG units.

In some embodiments, the hydrophilic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on an adapter and the carboxylic acid or activated carboxylic acid can be coupled to an amine on another adapter or linker with or without the use of a coupling reagent. Any appropriate coupling agent known to one skilled in the art can be used to couple the carboxylic acid with the amine such as, for example, DCC, DIC, HATU, HBTU, TBTU, and other activating agents described herein. In some embodiments, the linking group is maleimido-polymer (0.1-2.5 kDa)-COOH, iodoacetyl-polymer (0.1-2.5 kDa)-COOH, maleimido-polymer (0.1-2.5 kDa)-NHS, or iodoacetyl-polymer (0.1-2.5 kDa)-NHS.

In some embodiments, the linker comprises an amino acid, a dipeptide, a tripeptide, or a polypeptide, wherein the amino acid, dipeptide, tripeptide, or polypeptide comprises at least two activating groups, as described herein. In some embodiments, the linker comprises a moiety selected from the group consisting of: amino, ether, thioether, maleimido, disulfide, amide, ester, thioester, alkene, cycloalkene, alkyne, triazole, carbamate, carbonate, cathepsin B-cleavable, and hydrazone.

In some embodiments, the linker comprises a chain of atoms from 1 to about 60, 1 to about 30, 10 to 20, 2 to 10, 2 to 5, or 5 to 10 atoms long. In some embodiments, the chain atoms are all carbon atoms. In some embodiments, the chain atoms in the backbone of the linker are selected from the group consisting of C, O, N, and S. Chain atoms and linkers in some instances are selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

In some instances, a suitable polymer backbone has the formula X-polymer-L-Y, wherein polymer is poly(ethylene glycol), X is a functional group which does not react with azide groups, and Y is a suitable leaving group. Examples of suitable functional groups include, but are not limited to, hydroxyl, protected hydroxyl, acetal, alkenyl, amine, aminooxy, protected amine, protected hydrazide, protected thiol, carboxylic acid, protected carboxylic acid, maleimide, dithiopyridine, and vinylpyridine, and ketone. Examples of suitable leaving groups include, but are not limited to, chloride, bromide, iodide, mesylate, tresylate, and tosylate.

The linker may have a wide range of molecular weight or molecular length. Larger or smaller molecular weight linkers may be used to provide a desired spatial relationship or conformation between an adapter and the linked entity (e.g., a second adapter). Linkers having longer or shorter molecular length may also be used to provide a desired space or flexibility between an adapter and the linked entity.

In some embodiments, a linker comprises a water-soluble bifunctional linker that have a dumbbell structure that includes: a) an azide, an alkyne, a hydrazine, a hydrazide, a hydroxylamine, or a carbonyl-containing moiety on at least a first end of a polymer backbone; and b) at least a second functional group on a second end of the polymer backbone. The second functional group can be the same or different as the first functional group. The second functional group, in some embodiments, is not reactive with the first functional group. In some embodiments, water-soluble compounds comprise at least one arm of a branched molecular structure. For example, the branched molecular structure can be dendritic.

In exemplary embodiments, the polymer is linked to an adapter through a linker. For example, the linker can comprise one or two amino acids which at one end bind to the polymer (such as an albumin binding moiety) and at the other end bind to any available position on the polypeptide backbone. Additional exemplary linkers include a hydrophilic linker such as a chemical moiety which comprises at least 5 non-hydrogen atoms where 30-50% of these are either N or O.

In some embodiments, the adapters are joined by a polypeptide linker. In some embodiments, the polypeptide linker is one or more (e.g., 1, 1-2, 1-3, 1-4, 1-5, 1-6, 1-7, 1-8, 1-9, 1-10, 1-11, 1-12) amino acids in length, or longer in length.

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile. Non-limiting examples of carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other non-limiting examples of carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile. Non-limiting examples of non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C-X-C), wherein X is a heteroatom, including, but not limited to, oxygen, sulfur, or nitrogen.

In some cases, a polymer used herein terminates on one end with hydroxy or methoxy, i.e., X is H or CH₃ (“methoxy PEG”). Alternatively, the polymer can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester) and aldehydes) as well as functional groups that are inert to the 20 common amino acids but that react specifically with complementary functional groups (including but not limited to, azide groups, alkyne groups). It is noted that the other end of the polymer, which is shown in the above formula by Y, will attach either directly or indirectly to an adapter. Alternatively, an alkyne group on a polymer can be reacted with an azide group present on an adapter. In some embodiments, a strong nucleophile (including but not limited to, hydrazine, hydrazide, hydroxylamine, semicarbazide) can be reacted with an aldehyde or ketone group present on an adapter to form a hydrazone, oxime or semicarbazone, as applicable, which in some cases can be further reduced by treatment with an appropriate reducing agent. Alternatively, the strong nucleophile can be incorporated into the adapter and used to react preferentially with a ketone or aldehyde group present in the water-soluble polymer.

The activated ester of the carboxylic acid can be, for example, N-hydroxysuccinimide (NHS), tosylate (Tos), mesylate, triflate, a carbodiimide, or a hexafluorophosphate. In some embodiments, the carbodiimide is 1,3-dicyclohexylcarbodiimide (DCC), 1,1′-carbonyldiimidazole (CDI), 1-ethyl-3-(3-dimethylaminopropyl) carbodiimide hydrochloride (EDC), or 1,3-diisopropylcarbodiimide (DICD). In some embodiments, the hexafluorophosphate is selected from a group consisting of hexafluorophosphate benzotriazol-1-yl-oxy-tris(dimethylamino)phosphonium hexafluorophosphate (BOP), benzotriazol-1-yl-oxytripyrrolidinophosphonium hexafluorophosphate (PyBOP), 2-(1H-7-azabenzotriazol-1-yl)-1,1,3,3-tetramethyl uronium hexafluorophosphate (HATU), and o-benzotriazole-N,N,N′,N′-tetramethyl-uronium-hexafluoro-phosphate (HBTU).

Any molecular mass for a polymer can be used as practically desired, including but not limited to, from about 0.1 Daltons (Da) to 2,500 Da or more. The molecular weight of polymer may be of a wide range, including but not limited to, between about 100 Da and about 5,000 Da or more. In some instances the polymer is 50-5000 Da, 50-3000 Da, 50-2500 Da, 100-2500 Da, 250-2500 Da, 250-5000 Da, or 500-5000 Da. Branched chain polymers, including but not limited to, polymer molecules with each chain having a molecular weight ranging from 0.1-5 kDa, 0.1-4 kDa, 0.1-3 kDa, 0.1-2.5 kDa, 0.1-1.5 kDa.

Polymers may comprise azide- and acetylene-containing polymer derivatives comprising a water-soluble polymer backbone having an average molecular weight from about 800 Da to about 100,000 Da. The polymer backbone of the water-soluble polymer can be poly(ethylene glycol). However, it should be understood that a wide variety of water-soluble polymers including but not limited to poly(ethylene)glycol and other related polymers, including poly(dextran) and poly(propylene glycol), are also and that the use of the term PEG or poly(ethylene glycol) is intended to encompass and include all such molecules. The term PEG includes, but is not limited to, poly(ethylene glycol) in any of its forms, including bifunctional PEG, multiarmed PEG, derivatized PEG, forked PEG, branched PEG, pendent PEG (i.e. PEG or related polymers having one or more functional groups pendent to the polymer backbone), or PEG with degradable linkages therein.

In addition to these forms of polymer, the polymer can also be prepared with weak or degradable linkages in the backbone. For example, polymer can be prepared with ester linkages in the polymer backbone that are subject to hydrolysis. As shown below, this hydrolysis results in cleavage of the polymer into fragments of lower molecular weight: -polymer-CO₂-polymer-+H₂O àpolymer-CO₂H+HO-polymer-

Linkers may comprise a polymer, such as those comprising a water soluble backbone. In some embodiments, polymer backbones that are water-soluble comprise from 2 to about 300 termini. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da. The molecular weight of each chain of the polymer backbone may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of each chain of the polymer backbone is between about 100 Da and about 50,000 Da. In some embodiments, the molecular weight of each chain of the polymer backbone is between about 100 Da and about 40,000 Da. In some embodiments, the molecular weight of each chain of the polymer backbone is between about 1,000 Da and about 40,000 Da. In some embodiments, the molecular weight of each chain of the polymer backbone is between about 5,000 Da and about 40,000 Da. In some embodiments, the molecular weight of each chain of the polymer backbone is between about 10,000 Da and about 40,000 Da.

In some instances, adapters are linked via a water soluble polymer via methods described herein. In some embodiments, the method comprises contacting an adapter comprising a reactive amino acid side chain with a linker. In some instances a conjugate is synthesized by reacting a functional group present on an adapter with a reactive group present on the linker. In some instances an adapter conjugate is synthesized by reacting a functional group present on the linker with a reactive group present on the adapter.

In some embodiments, the linker has a molecular weight of 0.1 kDa to 5 kDa. In some embodiments, the linker has a molecular weight of 0.1 kDa to 2.5 kDa. In some embodiments, the linker or polymer is linear, branched, multimeric, or dendrimeric. In some embodiments, the linker or polymer is a bifunctional or multifunctional linker or a bifunctional or multifunctional polymer.

In other embodiments, the polymer is a water-soluble polymer. In other embodiments, the water-soluble polymer is polyethylene glycol (PEG). In some embodiments, the PEG has a molecular weight between 0.1 kDa and 10 kDa. In other embodiments, the PEG has a molecular weight between 0.1 kDa and 5 kDa. In other embodiments, the PEG has a molecular weight between 0.1 kDa and 4 kDa. In other embodiments, the PEG has a molecular weight between 0.1 kDa and 3 kDa. In other embodiments, the PEG has a molecular weight between 0.1 kDa and 2 kDa. In other embodiments, the PEG has a molecular weight between 0.1 kDa and 2.5 kDa. In some embodiments, the poly(ethylene glycol) molecule has a molecular weight of about 0.1 kDa to about 10 kDa. In some embodiments, the poly(ethylene glycol) molecule has a molecular weight of 0.1 kDa to 50 kDa. In some embodiments, the poly(ethylene glycol) has a molecular weight of 0.1 kDa to 2.5 kDa, or 0.2 to 2.2 kDa, or between 0.5 kDa and 2 kDa. For example, the molecular weight of the poly(ethylene glycol) polymer in some instances is about 0.5 kDa, or about 1 kDa, or about 2 kDa, or about 2.5 kDa. For example, the molecular weight of the poly(ethylene glycol) polymer in some instances is 0.1 kDa or 0.5 kDa or 1 kDa, or 2.5 kDa. In some embodiments the poly(ethylene glycol) molecule is a branched PEG. In some embodiments the poly(ethylene glycol) molecule is a branched 1K PEG. In some embodiments the poly(ethylene glycol) molecule is a branched 2.5K PEG. In some embodiments the poly(ethylene glycol) molecule is a branched 5K PEG. In some embodiments the poly(ethylene glycol) molecule is a linear PEG. In some embodiments the poly(ethylene glycol) molecule is a linear 2.5K PEG. In some embodiments the poly(ethylene glycol) molecule is a linear 10K PEG. In some embodiments the poly(ethylene glycol) molecule is a linear 2K PEG. In some embodiments the poly(ethylene glycol) molecule is a linear 0.5K PEG. In some embodiments, the molecular weight of the poly(ethylene glycol) polymer is an average molecular weight. In certain embodiments, the average molecular weight is the number average molecular weight (Mn). The average molecular weight may be determined or measured using GPC or SEC, SDS/PAGE analysis, RP-HPLC, mass spectrometry, or capillary electrophoresis.

Linkers may comprise cleavable bases. In some instances, cleavable bases may be removed to disconnect one or more adapters or portions thereof from each other. In some instances, a cleavable base comprises a base which may be enzymatically removed. In some instances, the cleavable base is uracil. In some instances, the cleavable base is removable with USER. A linker described herein may comprise nucleotide analogs that are recognized by specific enzymes. In some instances, a support linker comprises a nucleotide analog. In some instances, the support linker comprises deoxy uridine or 8-oxo-deoxyguanosine that are recognized by specific glycosylases (e.g., uracil deoxyglycosylase followed by endonuclease VIII, and 8-oxoguanine DNA glycosylase, respectively). In some embodiments, cleavage by glycosylases and/or endonucleases may require a double stranded DNA substrate. In some embodiments, support linkers comprise base analogs cleavable by endonuclease III which include, but are not limited to, urea, thymine glycol, methyl tartonyl urea, alloxan, uracil glycol, 6-hydroxy-5,6-dihydrocytosine, 5-hydroxyhydantoin, 5-hydroxycytocine, trans-1-carbamoyl-2-oxo-4, 5-dihydrooxyimidazolidine, 5,6-dihydrouracil, 5-hydroxy cytosine, 5-hydroxyuracil, 5-hydroxy-6-hydrouracil, 5-hydroxy-6-hydrothymine, 5,6-dihydrothymine. In some embodiments, support linkers comprise base analogs cleavable by formamidopyrimidine DNA glycosylase which include, but are not limited to, 7,8-dihydro-8-oxoguanine, 7,8-dihydro-8-oxoinosine, 7,8-dihydro-8-oxoadenine, 7,8-dihydro-8-oxonebularine, 4,6-diamino-5-formamidopyrimidine, 2,6-diamino-4-hydroxy-5-formamidopyrimidine, 2,6-diamino-4-hydroxy-5-N-methylformamidopyrimidine, 5-hydroxy cytosine, 5-hydroxyuracil. In some embodiments, support linkers comprise base analogs cleavable by hNeil 1 which include, but are not limited to, guanidinohydantoin, spiroiminodihydantoin, 5-hydroxyuracil, thymine glycol. In some embodiments, support linkers comprise base analogs cleavable by thymine DNA glycosylase which include, but are not limited to, 5-formylcytosine and 5-carboxycytosine. In some embodiments, In some embodiments, support linkers comprise base analogs cleavable by human alkyladenine DNA glycosylase which include, but are not limited to, 3-methyladenine, 3-methylguanine, 7-methylguanine, 7-(2-chloroehyl)-guanine, 7-(2-hydroxyethyl)-guanine, 7-(2-ethoxyethyl)-guanine, 1,2-bis-(7-guanyl) ethane, 1, N⁶-ethenoadenine, 1,N²-ethenoguanine, N²,3-ethenoguanine, N²,3-ethanoguanine, 5-formyluracil, 5-hydroxymethyluracil, hypoxanthine. In some embodiments, support linkers comprise 5-methylcytosine cleavable by 5-methylcytosine DNA glycosylase. In some instances, linkers comprise 1, 2, 3, 4, 5, 6, 8, 10, or more than 10 cleavable bases.

Linkers comprising polynucleotides may comprise a plurality of cleavable bases. In some instances, a plurality of cleavable bases comprise a restriction endonuclease (RE) site. Linkers may be cleaved by treatment with an appropriate endonuclease which recognizes the RE site. In some instances, an RE site is unique to the linker portion of the adapter conjugate. In some instances, treatment with a restriction enzyme does not cleave other portions of the conjugate, such as the adapter or sample nucleic acid (when ligated). In some instances, an RE site is present in both the linker and other portions of the adapter/sample nucleic acid, but these other portions are blocked from cleavage (e.g., methylated).

Linkers may be attached non-covalently. In some instances, complementary overlap regions of one or both adapters, and/or the linker are used for attachment. In some instances, a linker comprises a duplex region. In some instances, a duplex region comprises strands of at least partially complementary nucleic acids. Such linkers may be removed/cleaved by denaturation. In some instances, a linker is formed from at least a portion of a first adapter and a portion of a second adapter overlap. In some instances, one or more splint polynucleotides is used to generate a linker. In some instances, at least one splint polynucleotide is at least partially complementary to a portion of the first polynucleotide adapter or the second polynucleotide adapter. In some instances, a linker comprises at least two splint polynucleotides, wherein the at least two splint polynucleotides are at least partially overlapped with each other. In some instances, a linker comprises at least two splint polynucleotides, wherein the at least two splint polynucleotides are at least partially overlapped with a portion of the first polynucleotide adapter or the second polynucleotide adapter.

Provided herein are adapter-ligated samples. In some instances, an adapter-ligated sample comprises one or more adapters and a sample polynucleotide. In some instances, at least one sample polynucleotide is attached to the 3′ end of the first strand and the 5′ end of the second strand. In some instances, the at least one sample polynucleotide is attached to the 3′ end of the third strand and the 5′ end of the fourth strand. In some instances, the sample polynucleotides comprise genomic DNA. In some instances, the sample polynucleotides comprise cDNA. In some instances, the sample polynucleotides comprise cDNA. In some instances, further provided herein are libraries of adapter-ligated samples. In some instances, a library comprises a plurality of adapter-ligated samples. In some instances, each library is obtained from a different sample. Further provided herein are sequencing libraries comprising a plurality of libraries.

In some embodiments, sequencing libraries may comprise or be derived from different amounts of sample input (sample polynucleotides). In some instances, use of adapter conjugates described herein results in normalization of sample amount input. In some instances, no more than 10, 20, 25, 30, 35, 40, 45, 50, 55, 60, 65, 70, 75, 80, 85, 90, or no more than 95% of the sample polynucleotides are within one standard deviation of the mean sample polynucleotide amount. In some instances, no more than 10-95%, 10-90, 10-75, 10-60, 10-50, 20-95, 20-97, 40-95, 50-95, 75-99, 75-90 or 50-99% of the sample polynucleotides are within one standard deviation of the mean sample polynucleotide amount.

After adapters are generated, they may be resuspended. In some embodiments, an adapter may be resuspended in a buffer. In some embodiments, the buffer comprises tris-HCl and EDTA·Na₂ (“TE buffer”). In some embodiments, the TE buffer is at a 1× concentration. In some embodiments, an adapter is resuspended in 1× TE buffer to a concentration of about 100 UM and blended to reach a final working concentration of about 10 μM.

### Adapter Systems and Sample Normalization

Provided herein are adapter systems capable of library size normalization without additional steps, such as additional dilution or enzymatic processing, and methods of using the same. In some embodiments, the incorporation of inline barcodes into the adaptor system enables increased multiplexing capacity of the barcode systems for high-throughput library preparation.

Further provided herein are methods of sample normalization using the adapters described herein. In some instances, adapters comprise adapter conjugates. In some instances, a plurality of samples are processed for sequencing. In some instances, at least some of the plurality of samples are from different sources. In some instances, at least some of the plurality of samples comprise different amounts of sample nucleic acids. In some instances, sample nucleic acids are subjected to one or more steps of shearing/fragmentation (mechanical, or using amplification), end repair, a-tailing, ligation to adapter conjugates described herein, enrichment/capture, amplification, and sequencing. In some instances, a method comprises one or more steps of (a) providing a plurality of sample polynucleotides; and (b) ligating at least one composition described herein (e.g., adapter conjugate) to at least one sample polynucleotide. In some instances, the sample polynucleotides comprise genomic DNA. In some instances, the sample polynucleotides comprise cDNA. In some instances, the molar ratio of adapter conjugates to the plurality of sample polynucleotides is no more than 1:10, 1:5, 1:2, 1:1, 2:1, 3:1, 4:1, 5:1, 6:1, 10:1, 15:1, 20:1 or no more than 50:1. In some instances, the molar ratio of adapter conjugates to the plurality of sample polynucleotides is no more than 1:10-10:1, 1:10-5:1, 1:10-3:1, 1:10-2:1, 1:10-1:1, 1:5-10:1, 1:5-5:1, 1:5-2:1, 1:5-1:1, 1:3-1:5, and 1:3-1:1. In some instances, ligating occurs with an efficiency of at least 75%, 80%, 85%, 90%, 92%, 95%, 97%, 98%, or at least 99%. In some instances, ligating occurs with an efficiency of 75-99%, 80-99%, 85-99%, 90-99%, 90-95%, or 95-99%. In some instances, a plurality of sample polynucleotides represent a sequencing library. In some instances, use of adapter conjugates results in normalized representation of signal during sequencing for two or more samples.

Multiplexing gDNA on a sequencer may have improved efficiency and accuracy when the sequencing load is balanced evenly across all samples in terms of number of molecules. For example, some samples may include a relatively high concentration of gDNA, while other samples may include a relatively low concentration of gDNA. Yield and accuracy of sequencing results may be improved, and errors in sequencing may be reduced, by the normalization of sequencing load.

In conventional methods, a large excess of adapters is used for library construction. Because conventional methods use two independent ligation products per molecule, conversion cannot be easily controlled by modifying adapter concentration. In high-throughput settings the inability to control conversion by modifying adapter concentration may result in challenges which require qPCR and dilution for each sample.

The present disclosure describes systems and methods including hybrid-circular adapters (FIG. 1A) as opposed to conventional adaptors (FIG. 1B). The hybrid-circular adapters are generally compatible with standard sequencing workflows, without substantial modification to the workflow. By linking two adapters, two independent ligation events are replaced with a slower intermolecular ligation and a fast intramolecular ligation. In some embodiments, steric interactions of the adapters may be reduced by a flexible linker. Unlike conventional adapters, ligation conversion may be controlled by the number of adapter molecules to normalize sample amounts.

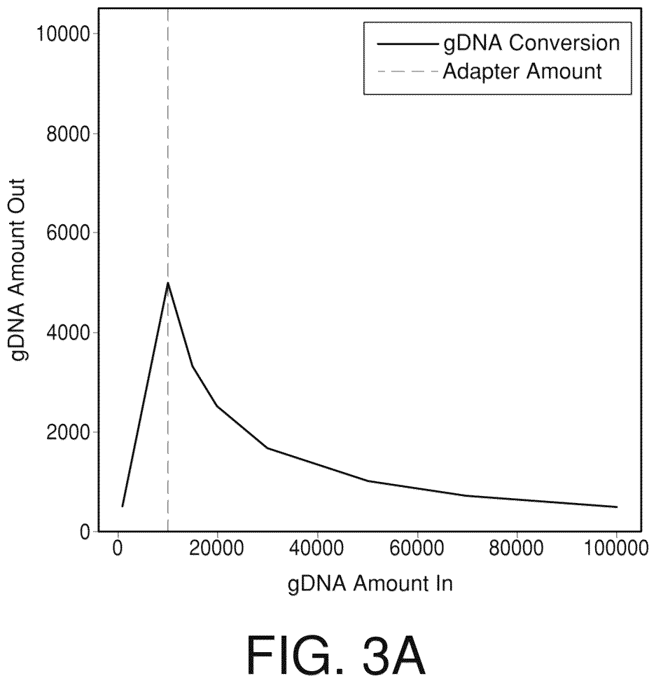

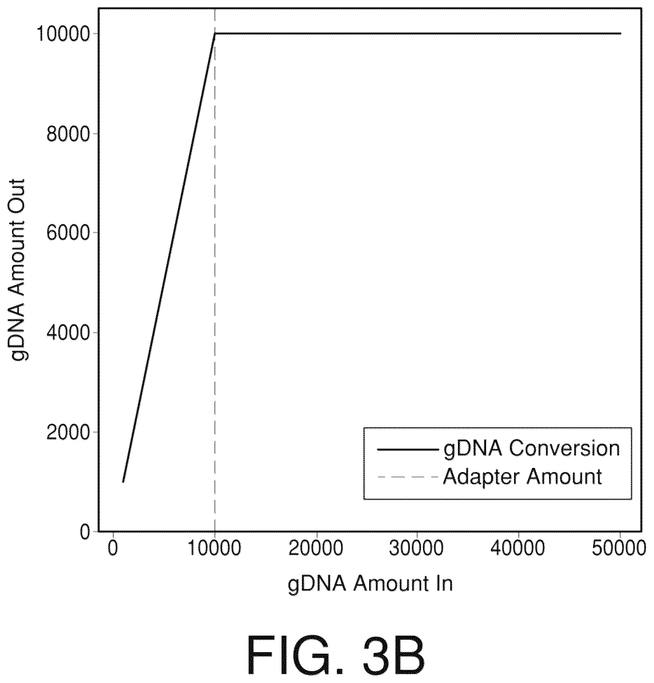

A comparison of ligation conversion between conventional adapters (FIG. 3A) and adapter systems of the present disclosure (FIG. 3B) is shown in the appendix. Additional examples, features, and functions of hybrid-circular adapters are described in U.S. Provisional Patent Application No. 63/511,086.

In some embodiments suitable for a high-throughput environment, more than about 1024 fragmented gDNA samples can be ligated to an equal concentration of hybrid-circular adapters generate adapter-ligated polynucleotide libraries for each sample. The concentration of hybrid-circular adapters may be adjusted based on the minimum sample concentration. In some examples, samples are enriched (e.g., exome enrichment), amplified, and/or subjected to next generation sequencing. The extent of signal normalization (e.g., counts) may be measured.

### Hybridization and Capture

Polynucleotide libraries may be designed to comprise polynucleotide sequences which are identical to or complementary (to target, hybridize) to one or more variants. In some instances, at least some of the polynucleotide molecules are each configured to hybridize to genomic regions which comprise at least two variants. In some instances, at least some of the polynucleotide molecules are each configured to hybridize to genomic regions which comprise at least one, two, three, four, five, six, or more than six variants. In some instances, at least some of the polynucleotides are each configured to hybridize to genomic regions which comprise one to four variants. In some instances, at least some of the polynucleotides are each configured to hybridize to genomic regions which comprise one to two or three variants. In some instances, at least 50% of the polynucleotides are each configured to hybridize to genomic regions which comprise at least two variants. In some instances, at least 50% of the polynucleotides are each configured to hybridize to genomic regions which comprise at least one, two, three, four, five, six, or more than six variants. In some instances, at least 50% of the polynucleotides are each configured to hybridize to genomic regions which comprise one to four variants. In some instances, at least 50% of the polynucleotides are each configured to hybridize to genomic regions which comprise one to two or three variants. In some instances, at least 25% of the polynucleotides are each configured to hybridize to genomic regions which comprise at least two variants. In some instances, at least 25% of the polynucleotides are each configured to hybridize to genomic regions which comprise at least one, two, three, four, five, six, or more than six variants. In some instances, at least 25% of the polynucleotides are each configured to hybridize to genomic regions which comprise one to four variants. In some instances, at least 25% of the polynucleotides are each configured to hybridize to genomic regions which comprise one to two or three variants. In some instances, at least 5% of the polynucleotides are each configured to hybridize to genomic regions which comprise at least two variants. In some instances, at least 5% of the polynucleotides are each configured to hybridize to genomic regions which comprise at least one, two, three, four, five, six, or more than six variants. In some instances, at least 5% of the polynucleotides are each configured to hybridize to genomic regions which comprise one to four variants. In some instances, at least 5% of the polynucleotides are each configured to hybridize to genomic regions which comprise one to two or three variants.

Polynucleotide libraries may be configured to bind to many variants. In some instances, a polynucleotide library is collectively configured to bind to genomic regions comprising about 50, 100, 200, 500, 800, 1000, 2000, 5000, 8000, 10,000, 20,000, 50,000, 80,000, 100,000, 250,000, 500,000, 750,000, 1 million, 1.5 million, 2 million, 2.5 million, 3 million, 3.5 million, 4 million, 4.5 million, or about 5 million variants. In some instances, a polynucleotide library is collectively configured to bind to genomic regions comprising at least 50, 100, 200, 500, 800, 1000, 2000, 5000, 8000, 10,000, 20,000, 50,000, 80,000, 100,000, 250,000, 500,000, 750,000, 1 million, 1.5 million, 2 million, 2.5 million, 3 million, 3.5 million, 4 million, 4.5 million, or at least 5 million variants. In some instances, a polynucleotide library is collectively configured to bind to genomic regions comprising 100-1000, 50-100, 50-500, 50-5000, 50-10,000, 100,000-5 million, 250,000-3 million, 500,000-2 million, 750,000-4 million, 1 million-5 million, 1 million-3 million, 1 million-4 million, or 4 million to 6 million variants.

Polynucleotide libraries for identifying variants may be optimized. In some instances, the library is uniform (each unique polynucleotide is equally represented). In some instances, the library is not uniform. In some instances, polynucleotides are represented in an amount within at least about 1.5 times the mean representation for the polynucleotide library. In some instances, polynucleotides are represented in an amount within at least about 2 times the mean representation for the polynucleotide library. In some instances, polynucleotides are represented in an amount within at least about 1.2 times the mean representation for the polynucleotide library. In some instances, polynucleotides are represented in an amount within at least about 1.7 times the mean representation for the polynucleotide library. In some instances, at least 80% polynucleotides are represented in an amount within at least about 1.5 times the mean representation for the polynucleotide library. In some instances, at least 80% polynucleotides are represented in an amount within at least about 2 times the mean representation for the polynucleotide library. In some instances, at least 80% polynucleotides are represented in an amount within at least about 1.7 times the mean representation for the polynucleotide library. In some instances, at least 80% polynucleotides are represented in an amount within at least about 2 times the mean representation for the polynucleotide library. In some instances, at least 90% polynucleotides are represented in an amount within at least about 1.5 times the mean representation for the polynucleotide library. In some instances, at least 90% polynucleotides are represented in an amount within at least about 2 times the mean representation for the polynucleotide library. In some instances, at least 80% polynucleotides are represented in an amount within at least about 1.7 times the mean representation for the polynucleotide library. In some instances, at least 90% polynucleotides are represented in an amount within at least about 2 times the mean representation for the polynucleotide library. In some instances, at least 95% polynucleotides are represented in an amount within at least about 1.5 times the mean representation for the polynucleotide library. In some instances, at least 95% polynucleotides are represented in an amount within at least about 2 times the mean representation for the polynucleotide library. In some instances, at least 95% polynucleotides are represented in an amount within at least about 1.7 times the mean representation for the polynucleotide library. In some instances, at least 95% polynucleotides are represented in an amount within at least about 2 times the mean representation for the polynucleotide library. Polynucleotide libraries in some instances comprise at least some polynucleotides which each comprise an overlap region with another polynucleotide in the library. In some instances at least 10%, 20%, 30%, 40%, 50%, 60%, 70%, 80%, or at least 90% of the polynucleotides each comprise an overlap region with another polynucleotide in the library. In some instances about 10%, 20%, 30%, 40%, 50%, 60%, 70%, 80%, or about 90% of the polynucleotides each comprise an overlap region with another polynucleotide in the library. In some instances 10%-90%, 10-80%, 10-75%, 25%-50%, 25-90%, 50-90%, 15-35%, or 80-99% of the polynucleotides each comprise an overlap region with another polynucleotide in the library. In some instances, the amount of at least some of the polynucleotides in the library is 5, 10, 20, 25, 50, 75, 100, 150, 200, 250, 300, 400, 500, or 600 times higher than the mean representation for the polynucleotide library. In some instances, the amount of at least 1% of the polynucleotides in the library is 5, 10, 20, 25, 50, 75, 100, 150, 200, 250, 300, 400, 500, or 600 times higher than the mean representation for the polynucleotide library. In some instances, the amount of at least 2% of the polynucleotides in the library is 5, 10, 20, 25, 50, 75, 100, 150, 200, 250, 300, 400, 500, or 600 times higher than the mean representation for the polynucleotide library. In some instances, the amount of at least 5% of the polynucleotides in the library is 5, 10, 20, 25, 50, 75, 100, 150, 200, 250, 300, 400, 500, or 600 times higher than the mean representation for the polynucleotide library. In some instances, the amount of no more than 5% of the polynucleotides in the library is 5, 10, 20, 25, 50, 75, 100, 150, 200, 250, 300, 400, 500, or 600 times higher than the mean representation for the polynucleotide library. In some instances, the amount of no more than 10% of the polynucleotides in the library is 5, 10, 20, 25, 50, 75, 100, 150, 200, 250, 300, 400, 500, or 600 times higher than the mean representation for the polynucleotide library. In some instances, the amount of at least 1%-10% of the polynucleotides in the library is 5, 10, 20, 25, 50, 75, 100, 150, 200, 250, 300, 400, 500, or 600 times higher than the mean representation for the polynucleotide library. In some instances, the amount of at least 1%-20% of the polynucleotides in the library is 5, 10, 20, 25, 50, 75, 100, 150, 200, 250, 300, 400, 500, or 600 times higher than the mean representation for the polynucleotide library. In some instances, the relative amount of a polynucleotide library is adjusted based on high or low GC content.

Polynucleotide libraries for identifying variants may collectively target a desired number of bases (bait territory). In some instances, a polynucleotide library comprise a bait territory of at least 5, 10, 15, 20, 25, 30, 40, 50, 60, 70, 80, 90 or at least 100 million bases. In some instances, a polynucleotide library comprise a bait territory of about 5, 10, 15, 20, 25, 30, 40, 50, 60, 70, 80, 90 or about 100 million bases. In some instances, a polynucleotide library comprise a bait territory of no more than 5, 10, 15, 20, 25, 30, 40, 50, 60, 70, 80, 90 or no more than 100 million bases.

Provided herein are systems and methods for generating polynucleotide libraries, such as those targeting variants. In some instances, systems comprise generation of in silico polynucleotide libraries comprising sequences. In some instances, systems generate nucleic acid standards described herein. In some instances, systems for generating a polynucleotide library comprise: a computing system comprising at least one processor and instructions executable by the at least one processor to perform operations comprising one or more of: (a) receiving as input; (b) generating a polynucleotide library by saturating at least one target region with one or more polynucleotides; and (c) generating one or more outputs comprising sequences of the polynucleotide library. In some instances the input comprises a nucleic acid reference sequence, one or more of at least one target region, a nucleic acid reference sequence, and one or more variables. In some instances an input nucleic reference sequence comprises a genome. In some instances an input nucleic reference sequence comprises mRNA. In some instances, the at least one target region comprises at least one exon. In some instances, the at least one target region comprises a variant. In some instances the variant comprises a single nucleotide variant (SNV), insertion/deletion (indel), or structural variant (SV).

Variables which control the sequences generated by systems may be tuned for specific applications or target regions. In some instances one or more variables independently comprise polynucleotide length, offset, number of probes, overlap, overhang, target region merges, and tiling depth. In some instances, the relationship between the size of the polynucleotide and the target is used to generate the library. In some instances the target region is smaller than a polynucleotide length. In some instances when the target region is smaller than a polynucleotide length, polynucleotides are generated with 1, 2, 3, 4, 5, or 6 base offsets. In some instances the target region is larger than a polynucleotide length. In some instances when the target region is larger than the polynucleotide length, polynucleotides are generated such that the entire target region is evenly covered.

### Unique Molecular Identifiers (UMIs)

Provided herein are adapters comprising unique molecular identifiers (UMIs). In some instances, adapters comprise universal adapters. In some instances, adapters comprise a Y-annealing region (anneals to form yoke), one or more Y-step non-annealing regions, a first index region, a second index region, a first UMI (index) region, a second UMI (index) region, and one or more regions exterior to the index. In some instances, adapters are ligated to sample polynucleotides to form an adapter-ligated polynucleotide. After denaturation of, top and bottom strand ligation products are formed. In some instances, each strand is labeled with a different UMI. After amplification with forward and backward primers, top strand and bottom strand PCR products are generated. In some instances, adapter ligated polynucleotides generated with universal adapters are further amplified with barcoded primers. In some instances adapters described herein comprise “in-line” UMIs, wherein at least one of a 5′ or 3′ UMI is not complementary to the other corresponding strand of the adapter. In some instances adapters described herein comprise “duplex” UMIs, wherein at least one of a 5′ or 3′ UMI is complementary to the other corresponding strand of the adapter.

Adapter-ligated libraries comprising unique molecular identifiers may be used to distinguish between “true” mutations from a polynucleotide sample library and artifacts generated during sequencing library preparation (e.g., PCR errors, sequencing errors, or other erroneous base call). In some instances, a workflow is used to analyze a library of adapter-ligated sample polynucleotides. Adapter-ligated sample polynucleotides each comprise two distinct UMIs represented by letters (A-F; six combinations of barcodes are shown for simplicity), and are attached to a sample polynucleotide. After sequencing, forward and reverse read pairs from sequencing are sorted into read pair groups. Next, read pairs are grouped by barcode and barcode position. Single-stranded consensus sequences are then generated from each group of barcode-grouped read pairs. Errors from D-C, and F-E are identified, although the error in A-B remains. Finally, duplex consensus sequences are generated by comparing each set of single stranded consensus sequences. The error in A-B can be identified, and true mutation E-F can be confirmed. In some instances, errors include substitutions, deletions, or insertions. In some instances, an error is present in the sample polynucleotide portion of an adapter-ligated polynucleotide. In some instances, an error is present in a barcode configured to identify a sample origin (e.g., index) or to uniquely identify a sample polynucleotide. In some instances, an error is present in a UMI. In some instances, an error is present in a sample index. Compositions and methods described herein in some instances are used to identify such errors.

Described herein are sets of UMIs, wherein the UMI sets have defined properties. In some instances, a UMI set comprises a plurality of different polynucleotides having unique sequences. In some instances, a UMI set is 8, 12, 16, 20, 24, 30, 32, 36, 39, 48, or 64 unique sequences. In some instances, the sequences of a UMI set differ by a Hamming distance of no more than 1, 2, 3, 4, or 5. In some instances, the sequences of a UMI set differ by a Hamming distance of at least 1, 2, 3, 4, or 5. In some instances, the sequences of a UMI set differ by a Hamming distance of at least 2. In some instances, the sequences of a UMI set differ by a Hamming distance of at least 1.

UMIs may be any length, depending on the desired application. In some instances, a UMI is no more than 15, 12, 10, 8, 7, 6, 5, 4, or not more than 3 bases in length. In some instances, a UMI is about 15, 12, 10, 8, 7, 6, 5, 4, or about 3 bases in length. In some instances, a UMI is about 3-12, 3-10, 3-8, 4-12, 4-10, 4-8, 6-12, or 8-12 bases in length. UMIs in a set may comprise more than one length. In some instances, 10, 20, 25, 30, 40, 50, 60, or 70 percent of UMIs in the set are a first length, and 90, 80, 75, 70, 60, 50, 40, or 30 percent are a second length. In some instances, the first length is 3-5 bases, and the second length is 3-5 bases. In some instances, UMIs comprise lengths of 5 or 6 bases.

After addition of UMI-containing adapters to sample polynucleotides, at least some of the sample polynucleotides may be uniquely labeled. In some instances, at least 30%, 50%, 75%, 80%, 90%, 95%, or at least 98% of the sample polynucleotides are ligated to adapters comprising UMIs. In some instances, at least 1%, 2%, 5%, 10%, 15%, 20%, 30%, 50%, 75%, 80%, 90%, 95%, or at least 98% of the sample polynucleotides are labeled with a unique UMI sequence. In some instances, no more than 1%, 2%, 5%, 10%, 15%, 20%, 30%, 50%, 75%, 80%, 90%, 95%, or no more than 98% of the sample polynucleotides are labeled with a unique UMI sequence. In some instances, at least 1%, 2%, 5%, 10%, 15%, 20%, 30%, 50%, 75%, 80%, 90%, 95%, or at least 98% of the sample polynucleotides are uniquely identifiable after labeling with a UMI.

In some embodiments, the UMI comprises one or more of the polynucleotide sequences AAGGA (SEQ ID NO: 1), ACAAC (SEQ ID NO: 2), ATACG (SEQ ID NO: 3), CACTG (SEQ ID NO: 4), CATGA (SEQ ID NO: 5), CGATA (SEQ ID NO: 6), CGTGT (SEQ ID NO: 7), GCCAT (SEQ ID NO: 8), GCTGT (SEQ ID NO: 9), GTCAC (SEQ ID NO: 10), GTCGT (SEQ ID NO: 11), TACGA (SEQ ID NO: 12), TCCTA (SEQ ID NO: 13), TCGTG (SEQ ID NO: 14), TGTCG (SEQ ID NO: 15), TTGGC (SEQ ID NO: 16), AACAC (SEQ ID NO: 17), AATGC (SEQ ID NO: 18), ACTAG (SEQ ID NO: 19), AGCAT (SEQ ID NO: 20), AGTAC (SEQ ID NO: 21), ATCTC (SEQ ID NO: 22), CAGAC (SEQ ID NO: 23), CAGTA (SEQ ID NO: 24), CGAAT (SEQ ID NO: 25), CGGTT (SEQ ID NO: 26), CTTGG (SEQ ID NO: 27), GCATA (SEQ ID NO: 28), GCTAA (SEQ ID NO: 29), GTGAG (SEQ ID NO: 30), GTGTC (SEQ ID NO: 31), and TGTGC (SEQ ID NO: 32). In some embodiments, the UMI comprises two or more of the polynucleotide sequences AAGGA (SEQ ID NO: 1), ACAAC (SEQ ID NO: 2), ATACG (SEQ ID NO: 3), CACTG (SEQ ID NO: 4), CATGA (SEQ ID NO: 5), CGATA (SEQ ID NO: 6), CGTGT (SEQ ID NO: 7), GCCAT (SEQ ID NO: 8), GCTGT (SEQ ID NO: 9), GTCAC (SEQ ID NO: 10), GTCGT (SEQ ID NO: 11), TACGA (SEQ ID NO: 12), TCCTA (SEQ ID NO: 13), TCGTG (SEQ ID NO: 14), TGTCG (SEQ ID NO: 15), TTGGC (SEQ ID NO: 16), AACAC (SEQ ID NO: 17), AATGC (SEQ ID NO: 18), ACTAG (SEQ ID NO: 19), AGCAT (SEQ ID NO: 20), AGTAC (SEQ ID NO: 21), ATCTC (SEQ ID NO: 22), CAGAC (SEQ ID NO: 23), CAGTA (SEQ ID NO: 24), CGAAT (SEQ ID NO: 25), CGGTT (SEQ ID NO: 26), CTTGG (SEQ ID NO: 27), GCATA (SEQ ID NO: 28), GCTAA (SEQ ID NO: 29), GTGAG (SEQ ID NO: 30), GTGTC (SEQ ID NO: 31), and TGTGC (SEQ ID NO: 32). In some embodiments, the UMI comprises five or more of the polynucleotide sequences AAGGA (SEQ ID NO: 1), ACAAC (SEQ ID NO: 2), ATACG (SEQ ID NO: 3), CACTG (SEQ ID NO: 4), CATGA (SEQ ID NO: 5), CGATA (SEQ ID NO: 6), CGTGT (SEQ ID NO: 7), GCCAT (SEQ ID NO: 8), GCTGT (SEQ ID NO: 9), GTCAC (SEQ ID NO: 10), GTCGT (SEQ ID NO: 11), TACGA (SEQ ID NO: 12), TCCTA (SEQ ID NO: 13), TCGTG (SEQ ID NO: 14), TGTCG (SEQ ID NO: 15), TTGGC (SEQ ID NO: 16), AACAC (SEQ ID NO: 17), AATGC (SEQ ID NO: 18), ACTAG (SEQ ID NO: 19), AGCAT (SEQ ID NO: 20), AGTAC (SEQ ID NO: 21), ATCTC (SEQ ID NO: 22), CAGAC (SEQ ID NO: 23), CAGTA (SEQ ID NO: 24), CGAAT (SEQ ID NO: 25), CGGTT (SEQ ID NO: 26), CTTGG (SEQ ID NO: 27), GCATA (SEQ ID NO: 28), GCTAA (SEQ ID NO: 29), GTGAG (SEQ ID NO: 30), GTGTC (SEQ ID NO: 31), and TGTGC (SEQ ID NO: 32). In some embodiments, the UMI comprises ten or more of the polynucleotide sequences AAGGA (SEQ ID NO: 1), ACAAC (SEQ ID NO: 2), ATACG (SEQ ID NO: 3), CACTG (SEQ ID NO: 4), CATGA (SEQ ID NO: 5), CGATA (SEQ ID NO: 6), CGTGT (SEQ ID NO: 7), GCCAT (SEQ ID NO: 8), GCTGT (SEQ ID NO: 9), GTCAC (SEQ ID NO: 10), GTCGT (SEQ ID NO: 11), TACGA (SEQ ID NO: 12), TCCTA (SEQ ID NO: 13), TCGTG (SEQ ID NO: 14), TGTCG (SEQ ID NO: 15), TTGGC (SEQ ID NO: 16), AACAC (SEQ ID NO: 17), AATGC (SEQ ID NO: 18), ACTAG (SEQ ID NO: 19), AGCAT (SEQ ID NO: 20), AGTAC (SEQ ID NO: 21), ATCTC (SEQ ID NO: 22), CAGAC (SEQ ID NO: 23), CAGTA (SEQ ID NO: 24), CGAAT (SEQ ID NO: 25), CGGTT (SEQ ID NO: 26), CTTGG (SEQ ID NO: 27), GCATA (SEQ ID NO: 28), GCTAA (SEQ ID NO: 29), GTGAG (SEQ ID NO: 30), GTGTC (SEQ ID NO: 31), and TGTGC (SEQ ID NO: 32).

UMIs may be represented at pre-selected percentages among a library of UMIs. In some instances at least 90% of the UMIs are present at fraction of 1-5%. In some instances at least 90% of the UMIs are present at fraction of 0.5%, 1%, 1.5%, 2%, 2.5%, 3%, 3.5%, 4%, 4.5%, 5%, 5.5%, 6%, 7%, or 8%. In some instances at least 90% of the UMIs are present at fraction of 0.5-8%, 1-7%, 1.5-7%, 2-7%, 2.5-6%, 3-8%, 3-6%, 1-5%, 0.5-5.5%, 1-4%, 1-6%, or 1-8%.

Any amount of sample polynucleotides (e.g., input DNA or other nucleic acid) may be ligated to adapters described herein. In some instances, the amount of sample polynucleotides is about 1, 5, 8, 10, 15, 20, 25, 30, 50, 75, or about 100 ng. In some instances, the amount of sample polynucleotides is no more than 1, 5, 8, 10, 15, 20, 25, 30, 50, 75, or no more than 100 ng. In some instances, the amount of sample polynucleotides is at least 1, 5, 8, 10, 15, 20, 25, 30, 50, 75, or at least 100 ng. In some instances, the amount of sample polynucleotides 1-10 ng, 1-100 ng, 3-10 ng, 5-100 ng, 5-75 ng, 5-50 ng, 10-100 ng, 10-50 ng, 25-100 ng, or 25-75 ng.

Provided herein are methods of generating adapters comprising UMIs. In a first method of adapter synthesis comprising synthesis of a top strand of an adapter comprising at least one UMI and a complementary bottom strand. After annealing the top and bottom adapter strands, an adapter comprising the structure of an adapter is formed. In a second method of adapter synthesis, a top strand is synthesized without a UMI, and a bottom strand comprising a complementary region and a UMI. After, annealing, PCR is used to generate a complementary UMI on the top strand, and a terminal transferase adds a T to the 3′ end of top strand to generate an adapter. In a third method of synthesis, a top strand which does not comprise a UMI, and a bottom strand comprising a UMI, a restrictions site, and a 5′ overhang are synthesized. After annealing, the top strand is extended with PCR, and a restriction endonuclease is used to cleave a portion of the 3′ top strand and 5′ bottom strand to generate an adapter. In a fourth method of adapter synthesis, two complementary strands each comprising a UMI, a restriction site, and an overhang portion (3′ top strand, 5′ bottom strand) are synthesized, annealed, and cleaved with a restriction enzyme to generate an adapter. More than one UMIs may be present per adapter. In some instances, an adapter comprises 1, 2, 3, 4, 5, or more UMIs. In some instances, adapters comprise a first UMI and a second UMI. In some instances, a first UMI and a second UMI are complementary. In some instances, adapters comprise a first UMI and a second UMI. In some instances, a first UMI and a second UMI are not complementary. In some instances adapters are combined into libraries of adapters. In some instances adapters in a library comprise UMIs. In some instances adapters in a library comprise unique combinations of a first UMI and a second UMI.

### Universal Adapters

Provided herein are universal adapters. In some instances, universal adapters comprise one or more unique molecular identifiers. In some instances, the universal adapters disclosed herein may comprise a universal polynucleotide adapter comprising a first strand and a second strand. In some instances, a first strand comprises a first primer binding region, a first non-complementary region, and a first yoke region. In some instances, a second strand comprises a second primer binding region, a second non-complementary region, and a second yoke region. In some instances, a primer binding region allows for PCR amplification of a polynucleotide adapter. In some instances, a primer binding region allows for PCR amplification of a polynucleotide adapter and concurrent addition of one or more barcodes to the polynucleotide adapter. In some instances, the first yoke region is complementary to the second yoke region. In some instances, the first non-complementary region is not complementary to the second non-complementary region. In some instances, the universal adapter is a Y-shaped or forked adapter. In some instances, one or more yoke regions comprise nucleobase analogues that raise the Tm between a first yoke region and a second yoke region. Primer binding regions as described herein may be in the form of a terminal adapter region of a polynucleotide. In some instances, a universal adapter comprises one index sequence. In some instances, a universal adapter comprises one unique molecular identifier. In some instances, universal adapters are configured for use with barcoded primers, wherein after ligation, barcoded primers are added via PCR.

A universal adapter comprises a polynucleotide sequence that may be shortened relative to the polynucleotide sequence of a typical barcoded adapter (e.g., full-length “Y adapter”). For example, a universal adapter may comprise a polynucleotide sequence that is 20-45 bases in length. In some instances, a universal adapter may comprise a polynucleotide sequence that is 25-40 bases in length. In some instances, a universal adapter may comprise a polynucleotide sequence that is 30-35 bases in length. In some instances, a universal adapter may comprise a polynucleotide sequence that is no more than 50 bases in length, no more than 45 bases in length, no more than 40 bases in length, no more than 35 bases in length, no more than 30 bases in length, or no more than 25 bases in length. In some instances, a universal adapter may comprise a polynucleotide sequence that is about 25, 27, 30, 32, 34, 36, 38, 40, 42, 44, 46, 48, 50, 52, 54, 56, 58, or 60 bases in length. In some instances, a universal adapter may comprise a polynucleotide sequence that is about 60 base pairs in length. In some instances, a universal adapter may comprise a polynucleotide sequence that is about 58 base pairs in length. In some instances, a universal adapter may comprise a polynucleotide sequence that is about 52 base pairs in length. In some instances, a universal adapter may comprise a polynucleotide sequence that is about 33 base pairs in length.

A universal adapter may be modified to facilitate ligation with a sample polynucleotide. For example, the 5′ terminus is phosphorylated. In some instances, a universal adapter comprises one or more non-native nucleobase linkages such as a phosphorothioate linkage. For example, a universal adapter comprises a phosphorothioate between the 3′ terminal base, and the base adjacent to the 3′ terminal base. A sample polynucleotide in some instances comprises nucleic acid from a variety of sources, such as DNA or RNA of human, bacterial, plant, animal, fungal, or viral origin. An adapter-ligated sample polynucleotide in some instances comprises a sample polynucleotide (e.g., sample nucleic acid) with adapters universal adapters ligated to both the 5′ and 3′ end of the sample polynucleotide to form an adapter-ligated polynucleotide. A duplex sample polynucleotide comprises both a first strand (forward) and a second strand (reverse).

Universal adapters may contain any number of different nucleobases (DNA, RNA, etc.), nucleobase analogues, or non-nucleobase linkers or spacers. For example, a universal adapter comprises one or more nucleobase analogues or other groups that enhance hybridization (Tₘ) between two polynucleotide strands of the universal adapter. In some instances, nucleobase analogues are present in the yoke region of a universal adapter. Nucleobase analogues and other groups include, but are not limited to, locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), xenonucleic acids (XNAs) morpholino backbone-modified bases, minor grove binders (MGBs), spermine, G-clamps, or a anthraquinone (Uaq) caps.

Universal adapters may comprise any number of nucleobase analogues (such as LNAs or BNAs), depending on the desired hybridization Tₘ. For example, a universal adapter comprises 1 to 20 nucleobase analogues. In some instances, a universal adapter comprises 1 to 8 nucleobase analogues. In some instances, an adapter comprises at least 1, 2, 3, 4, 5, 6, 7, 8, 9, 10, 11, 12, or at least 12 nucleobase analogues. In some instances, an adapter comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the adapter. For example, an adapter comprises at least 1%, 2%, 5%, 10%, 12%, 18%, 24%, 30%, or more than 30% nucleobase analogues. In some instances, universal adapters described herein comprise methylated nucleobases, such as methylated cytosine.

### Polynucleotide Barcodes

A barcode sequence (or indices sequence) is a defined, short polynucleotide sequence that can be used as a sample-specific label during PCR amplification. Polynucleotide primers may comprise barcode sequences (hereinafter, “barcodes”). Adapters, as described herein, may comprise one or more barcodes. In some instances, an adapter comprises at least one indexing barcode and at least one unique molecular identifier (UMI) barcode. Barcodes can be attached to universal adapters, for example, using PCR and barcoded primers to generate barcoded adapter-ligated sample polynucleotides. Primer binding sites, such as universal primer binding sites, facilitate simultaneous amplification of all members of a barcoded primer library, or a subpopulation of members. In some instances, a primer binding site comprises a primer linking region that binds to a flow cell or other solid support during next generation sequencing. In some instances, a barcoded primer comprises a P5 linking region or P7 linking region as shown in Table 1, below.

TABLE 1
Exemplary Primer Linking Regions

| Primer Linking | Polynucleotide Sequence |
|---|---|
| Region | (5′-3′) |
| P5 | AATGATACGGCGACCACCGA |
| | (SEQ ID NO: 33) |
| P7 | CAAGCAGAAGACGGCATACGAGAT |
| | (SEQ ID NO: 34) |

In some instances, primer binding sites are configured to bind to universal adapter sequences, and facilitate amplification and generation of barcoded adapters. In some instances, barcoded primers are no more than 60 bases in length. In some instances, barcoded primers are no more than 55 bases in length. In some instances, barcoded primers are 50-60 bases in length. In some instances, barcoded primers are about 60 bases in length. In some instances, barcodes described herein comprise methylated nucleobases, such as methylated cytosine.

The number of unique barcode sequences available for a barcode set (collection of unique barcodes or barcode combinations configured to be used together to unique define samples) may depend on length. In some instances, a Hamming distance is defined by the number of base differences between any two barcodes. In some instances, a Levenshtein distance is defined by the number changes needed to change one barcode into another (insertions, substitutions, or deletions). In some instances, barcode sets described herein comprise a Levenshtein distance of at least 2, 3, 4, 5, 6, 7, or at least 8. In some instances, barcode sets described herein comprise a Hamming distance of at least 2, 3, 4, 5, 6, 7, or at least 8.

Barcodes may be incorrectly associated with a different sample than they were assigned. In some instances, incorrect barcodes occur from PCR errors (e.g., substitution) during library amplification. In some instances, entire barcodes “hop” or are transferred from one polynucleotide sample to another. In some instances, such transfers result from cross-contamination of free adapters or primers during a library generation workflow. In some instances, a barcode set is chosen to minimize “barcode hopping”. In some instances, barcode hopping (for a single barcode) for a barcode set described herein is no more than 7%, 5%, 4%, 3%, 2%, 1%, 0.5%, or no more than 0.1%. In some instances, barcode hopping (for a single barcode) for a barcode set described herein is 0.1-6%, 0.1-5%, 0.2-5%, 0.5-5%, 1-7%, 1-5%, or 0.5-7%. In some instances, barcode hopping (for two barcodes) for a barcode set described herein is no more than 0.7%, 0.5%, 0.4%, 0.3%, 0.2%, 0.1%, 0.05%, or no more than 0.1%. In some instances, barcode hopping (for two barcodes) for a barcode set described herein is 0.01-0.6%, 0.01-0.5%, 0.02-0.5%, 0.05-0.5%, 0.1-0.7%, 0.1-0.5%, or 0.05-0.7%.

Barcoded primers comprise one or more barcodes. In some instances, the barcodes are added to universal adapters through PCR reaction. Barcodes are nucleic acid sequences that allow some feature of a polynucleotide with which the barcode is associated to be identified. In some instances, a barcode comprises an index sequence. In some instances, index sequences allow for identification of a sample, or unique source of nucleic acids to be sequenced. A barcode or combination of barcodes in some instances identifies a specific patient. A barcode or combination of barcodes in some instances identifies a specific sample from a patient among other samples from the same patient. After sequencing, the barcode (or barcode region) provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, a barcode is positioned on the 5′ and the 3′ sides of a sample polynucleotide. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, or more 512 barcoded libraries are used. In some instances, at least 400, 500, 800, 1000, 2000, 5000, 10,000, 12,000, 15,000, 18,000, 20,000, or at 25,000 barcodes are used.

Barcoded primers or adapters may comprise unique molecular identifiers (UMIs). In some instances, such UMIs uniquely tag all nucleic acids in a sample. In some instances, at least 60%, 70%, 80%, 90%, 95%, or more than 95% of the nucleic acids in a sample are tagged with a UMI. In some instances, at least 85%, 90%, 95%, 97%, or at least 99% of the nucleic acids in a sample are tagged with a unique barcode, or UMI. Barcoded primers in some instances comprise an index sequence and one or more UMI. UMIs allow for internal measurement of initial sample concentrations or stoichiometry prior to downstream sample processing (e.g., PCR or enrichment steps) which can introduce bias. In some instances, UMIs comprise one or more barcode sequences. In some instances, each strand (forward vs. reverse) of an adapter-ligated sample polynucleotide possesses one or more unique barcodes. Such barcodes are optionally used to uniquely tag each strand of a sample polynucleotide. In some instances, a barcoded primer comprises an index barcode and a UMI barcode. In some instances, after amplification with at least two barcoded primers, the resulting amplicons comprise two index sequences and two UMIs. In some instances, after amplification with at least two barcoded primers, the resulting amplicons comprise two index barcodes and one UMI barcode. In some instances, each strand of a universal adapter-sample polynucleotide duplex is tagged with a unique barcode, such as a UMI or index barcode.

Barcoded primers in a library comprise a region that is complementary to a primer binding region on a universal adapter. For example, universal adapter binding region is complementary to primer region of the universal adapter, and universal adapter binding region is complementary to primer region of the universal adapter. Such arrangements facilitate extension of universal adapters during PCR, and attach barcoded primers. In some instances, the Tm between the primer and the primer binding region is 40-65 degrees C. In some instances, the Tm between the primer and the primer binding region is 42-63 degrees C. In some instances, the Tm between the primer and the primer binding region is 50-60 degrees C. In some instances, the Tm between the primer and the primer binding region is 53-62 degrees C. In some instances, the Tm between the primer and the primer binding region is 54-58 degrees C. In some instances, the Tm between the primer and the primer binding region is 40-57 degrees C. In some instances, the Tm between the primer and the primer binding region is 40-50 degrees C. In some instances, the Tm between the primer and the primer binding region is about 40, 45, 47, 50, 52, 53, 55, 57, 59, 61, or 62 degrees C.

### Hybridization Blockers

Blockers may contain any number of different nucleobases (DNA, RNA, etc.), nucleobase analogues (non-canonical), or non-nucleobase linkers or spacers. In some instances, blockers comprise universal blockers. Such blockers may in some instances are described as a “set”, wherein the set comprises two or more blockers configured to prevent unwanted interactions with the same adapter sequence. In some instances, universal blockers prevent adapter-adapter interactions independent of one or more barcodes present on at least one of the adapters. For example, a blocker comprises one or more nucleobase analogues or other groups that enhance hybridization (Tm) between the blocker and the adapter. In some instances, a blocker comprises one or more nucleobases which decrease hybridization (Tm) between the blocker and the adapter (e.g., “universal” bases). In some instances, a blocker described herein comprises both one or more nucleobases which increase hybridization (Tm) between the blocker and the adapter and one or more nucleobases which decrease hybridization (Tm) between the blocker and the adapter.

Described herein are hybridization blockers comprising one or more regions which enhance binding to targeted sequences (e.g., adapter), and one or more regions which decrease binding to target sequences (e.g., adapter). In some instances, each region is tuned for a given desired level of off-bait activity during target enrichment applications. In some instances, each region can be altered with either a single type of chemical modification/moiety or multiple types to increase or decrease overall affinity of a molecule for a targeted sequence. In some instances, the melting temperature of all individual members of a blocker set are held above a specified temperature (e.g., with the addition of moieties such as LNAs and/or BNAs). In some instances, a given set of blockers will improve off bait performance independent of index length, independent of index sequence, and independent of how many adapter indices are present in hybridization.

Blockers may comprise moieties which increase and/or decrease affinity for a target sequencing, such as an adapter. In some instances, such specific regions can be thermodynamically tuned to specific melting temperatures to either avoid or increase the affinity for a particular targeted sequence. This combination of modifications is in some instances designed to help increase the affinity of the blocker molecule for specific and unique adapter sequence and decrease the affinity of the blocker molecule for repeated adapter sequence (e.g., Y-stem annealing portion of adapter). In some instances, blockers comprise moieties which decrease binding of a blocker to the Y-stem region of an adapter. In some instances, blockers comprise moieties which decrease binding of a blocker to the Y-stem region of an adapter, and moieties which increase binding of a blocker to non-Y-stem regions of an adapter.

Blockers (e.g., universal blockers) and adapters may form a number of different populations during hybridization. In a population ‘A’ in some instances comprises blockers correctly bound to non-index regions of the adapters. In a population ‘B’, a region of the blockers is bound to the “yoke” region of the adapter, but a remaining portion of the blocker does not bind to an adjacent region of the adapter. In a population ‘C’, two blockers unproductively dimerize. In a population ‘D’, blockers are unbound to any other nucleic acids. In some instances, when the number of DNA modifications that decrease affinity in the Y-stem annealing region of the blocker are increased, the populations ‘A’ & ‘D’ dominate and either have the desired or minimal effect. In some instances, as the number of DNA modifications that decrease affinity in the Y-stem annealing region of the blocker are decreased, the populations ‘B’ & ‘C’ dominate and have undesired effects where daisy-chaining or annealing to other adapters can occur (‘B’) or sequester blockers where they are unable to function properly (‘C’).

The index on both single or dual index adapter designs may be either partially or fully covered by universal blockers that have been extended with specifically designed DNA modifications to cover adapter index bases. In some instances, such modifications comprise moieties which decrease annealing to the index, such as universal bases. In some instances, the index of a dual index adapter is partially covered (or is overlapped) by one or more blockers. In some instances, the index of a dual index adapter is fully covered by one or more blockers. In some instances, the index of a single index adapter is partially covered by one or more blockers. In some instances, the index of a single index adapter is fully covered by one or more blockers. In some instances, a blocker overlaps an index sequence by at least 1, 2, 3, 4, 5, 6, 7, 8, 9, 10, 11, 12, 13, 14, 15, 20 or more than 20 bases. In some instances, a blocker overlaps an index sequence by no more than 1, 2, 3, 4, 5, 6, 7, 8, 9, 10, 11, 12, 13, 14, 15, 20, or no more than 25 bases. In some instances, a blocker overlaps an index sequence by about 1, 2, 3, 4, 5, 6, 7, 8, 9, 10, 11, 12, 13, 14, 15, 20 or about 30 bases. In some instances, a blocker overlaps an index sequence by 1-5, 1-3, 2-5, 2-8, 2-10, 3-6, 3-10, 4-10, 4-15, 1-4 or 5-7 bases. In some instances, a region of a blocker which overlaps an index sequences comprises at least one 2-deoxyinosine or 5-nitroindole nucleobase.

One or two blockers may overlap with an index sequence present on an adapter. In some instances, one or two blockers combined overlap with at least 1, 2, 3, 4, 5, 6, 7, 8, 9, 10, 11, 12, 13, 14, 15, 20 or more than 20 bases of the index sequence. In some instances, one or two blockers combined overlap with no more than 1, 2, 3, 4, 5, 6, 7, 8, 9, 10, 11, 12, 13, 14, 15, 20 or no more than 20 bases of the index sequence. In some instances, one or two blockers combined overlap with about 1, 2, 3, 4, 5, 6, 7, 8, 9, 10, 11, 12, 13, 14, 15, 20 or about 20 bases of the index sequence. In some instances, one or two blockers combined overlap by 1-5, 1-3, 2-5, 2-8, 2-10, 3-6, 3-10, 4-10, 4-15, 1-4 or 5-7 bases of the index sequence. In some instances, a region of a blocker which overlaps an index sequences comprises at least one 2-deoxyinosine or 5-nitroindole nucleobase.

In a first arrangement, the length of the adapter index overhang may be varied. When designed from a single side, the adapter index overhang can be altered to cover from 0 to n of the adapter index bases from either side of the index. This allows for the ability to design such adapter blockers for both single and dual index adapter systems.

In a second arrangement, the adapter index bases are covered from both sides. When adapter index bases are covered from both sides, the length of the covering region of each blocker can be chosen such that a single pair of blockers is capable of interacting with a range of adapter index lengths while still covering a significant portion of the total number of index bases. As an example, take two blockers that have been designed with 3 bp overhangs that cover the adapter index. In the context of 6 bp, 8 bp, or 10 bp adapter index lengths, these blockers will leave 0 bp, 2 bp, or 4 bp exposed during hybridization, respectively.

In a third arrangement, modified nucleobases are selected to cover index adapter bases. Examples of these modifications that are currently commercially available include degenerate bases (e.g., mixed bases of A, T, C, G), 2′-deoxyInosine, & 5-nitroindole.

In a forth arrangement, blockers with adapter index overhangs bind to either the sense (i.e., ‘top’) or anti-sense (i.e., ‘bottom’) strand of a next generation sequencing library.

In a fifth arrangement, blockers are further extended to cover other polynucleotide sequences (e.g., a poly-A tail added in a previous biochemical step in order to facilitate ligation or other method to introduce a defined adapter sequence, unique molecular identifier for bioinformatic assignment following sequencing, etc.) in addition to the standard adapter index bases of defined length and composition. These types of sequences can be placed in multiple locations of an adapter and in this case the most widely utilized case (e.g., unique molecular index next to the genomic insert) is presented. Other positions for the unique molecular identifier (e.g., next to adapter index bases) could also be addressed with similar approaches.

In a sixth arrangement, all of the previous arrangements are utilized in various combinations to meet a targeted performance metric for off-bait performance during target enrichment under specified conditions.

Blockers may comprise moieties, such as nucleobase analogues. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), inosine, 2′-deoxyInosine, 3-nitropyrrole, 5-nitroindole, xenonucleic acids (XNAs) morpholino backbone-modified bases, minor grove binders (MGBs), spermine, G-clamps, or a anthraquinone (Uaq) caps. In some instances, nucleobase analogues comprise universal bases, wherein the nucleobase has a lower Tm for binding to a cognate nucleobase. In some instances, universal bases comprise 5-nitroindole or 2′-deoxyInosine. In instances, blockers comprise spacer elements that connect two polynucleotide chains. In some instances, blockers comprise one or more nucleobase analogues. In some instances, such nucleobase analogues are added to control the Tm of a blocker. Blockers may comprise any number of nucleobase analogues (such as LNAs or BNAs), depending on the desired hybridization Tm. For example, a blocker comprises 20 to 40 nucleobase analogues. In some instances, a blocker comprises 8 to 16 nucleobase analogues. In some instances, a blocker comprises at least 1, 2, 3, 4, 5, 6, 7, 8, 9, 10, 11, 12, or at least 12 nucleobase analogues. In some instances, a blocker comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the blocker. For example, a blocker comprises at least 1%, 2%, 5%, 10%, 12%, 18%, 24%, 30%, or more than 30% nucleobase analogues. In some instances, the blocker comprising a nucleobase analogue raises the Tm in a range of about 2° C. to about 8° C. for each nucleobase analogue. In some instances, the Tm is raised by at least or about 1° C., 2° C., 3° C., 4° C., 5° C., 6° C., 7° C., 8° C., 9° C., 10° C., 12° C., 14° C., or 16° C. for each nucleobase analogue. Such blockers in some instances are configured to bind to the top or “sense” strand of an adapter. Blockers in some instances are configured to bind to the bottom or “anti-sense” strand of an adapter. In some instances a set of blockers includes sequences which are configured to bind to both top and bottom strands of an adapter. Additional blockers in some instances are configured to the complement, reverse, forward, or reverse complement of an adapter sequence. In some instances, a set of blockers targeting a top (binding to the top) or bottom strand (or both) is designed and tested, followed by optimization, such as replacing a top blocker with a bottom blocker, or a bottom blocker with a top blocker. In some instances, a blocker is configured to overlap fully or partially with bases of an index or barcode on an adapter. A set of blockers in some instances comprise at least one blocker overlapping with an adapter index sequence. A set of blockers in some instances comprise at least one blocker overlapping with an adapter index sequence, and at least one blocker which does not overlap with an adapter sequence. A set of blockers in some instances comprise at least one blocker which does not overlap with a yoke region sequence. A set of blockers in some instances comprise at least one blocker which does not overlap with a yoke region sequence and at least one blocker which overlaps with a yoke region sequence. A sets of blockers in some instances comprises 2, 3, 4, 5, 6, 7, 8, 9, 10, or more than 10 blockers.

Blockers may be any length, depending on the size of the adapter or hybridization Tm. For example, blockers are 20 to 50 bases in length. In some instances, blockers are 25 to 45 bases, 30 to 40 bases, 20 to 40 bases, or 30 to 50 bases in length. In some instances, blockers are 25 to 35 bases in length. In some instances blockers are at least 25, 26, 27, 28, 29, 30, 31, 32, 33, 34, or at least 35 bases in length. In some instances, blockers are no more than 25, 26, 27, 28, 29, 30, 31, 32, 33, 34, or no more than 35 bases in length. In some instances, blockers are about 25, 26, 27, 28, 29, 30, 31, 32, 33, 34, or about 35 bases in length. In some instances, blockers are about 50 bases in length. A set of blockers targeting an adapter-tagged genomic library fragment in some instances comprises blockers of more than one length. Two blockers are in some instances tethered together with a linker. Various linkers are well known in the art, and in some instances comprise alkyl groups, polyether groups, amine groups, amide groups, or other chemical group. In some instances, linkers comprise individual linker units, which are connected together (or attached to blocker polynucleotides) through a backbone such as phosphate, thiophosphate, amide, or other backbone. In an exemplary arrangement, a linker spans the index region between a first blocker that each targets the 5′ end of the adapter sequence and a second blocker that targets the 3′ end of the adapter sequence. In some instances, capping groups are added to the 5′ or 3′ end of the blocker to prevent downstream amplification. Capping groups variously comprise polyethers, polyalcohols, alkanes, or other non-hybridizable group that prevents amplification. Such groups are in some instances connected through phosphate, thiophosphate, amide, or other backbone. In some instances, one or more blockers are used. In some instances, at least 4 non-identical blockers are used. In some instances, a first blocker spans a first 3′ end of an adaptor sequence, a second blocker spans a first 5′ end of an adaptor sequence, a third blocker spans a second 3′ end of an adaptor sequence, and a fourth blockers spans a second 5′ end of an adaptor sequence. In some instances a first blocker is at least 20, 21, 22, 23, 24, 25, 26, 27, 28, 29, 30, 31, 32, 33, 34, or at least 35 bases in length. In some instances a second blocker is at least 20, 21, 22, 23, 24, 25, 26, 27, 28, 29, 30, 31, 32, 33, 34, or at least 35 bases in length. In some instances a third blocker is at least 20, 21, 22, 23, 24, 25, 26, 27, 28, 29, 30, 31, 32, 33, 34, or at least 35 bases in length. In some instances a fourth blocker is at least 20, 21, 22, 23, 24, 25, 26, 27, 28, 29, 30, 31, 32, 33, 34, or at least 35 bases in length. In some instances, a first blocker, second blocker, third blocker, or fourth blocker comprises a nucleobase analogue. In some instances, the nucleobase analogue is LNA.

The design of blockers may be influenced by the desired hybridization Tm to the adapter sequence. In some instances, non-canonical nucleic acids (for example locked nucleic acids, bridged nucleic acids, or other non-canonical nucleic acid or analog) are inserted into blockers to increase or decrease the blocker's Tm. In some instances, the Tm of a blocker is calculated using a tool specific to calculating Tm for polynucleotides comprising a non-canonical amino acid. In some instances, a Tm is calculated using the Exiqon™ online prediction tool. In some instances, blocker Tm described herein are calculated in-silico. In some instances, the blocker Tm is calculated in-silico, and is correlated to experimental in-vitro conditions. Without being bound by theory, an experimentally determined Tm may be further influenced by experimental parameters such as salt concentration, temperature, presence of additives, or other factor. In some instances, Tm described herein are in-silico determined Tm that are used to design or optimize blocker performance. In some instances, Tm values are predicted, estimated, or determined from melting curve analysis experiments. In some instances, blockers have a Tm of 70 degrees C. to 99 degrees C. In some instances, blockers have a Tm of 75 degrees C. to 90 degrees C. In some instances, blockers have a Tm of at least 85 degrees C. In some instances, blockers have a Tm of at least 70, 72, 75, 77, 80, 82, 85, 88, 90, or at least 92 degrees C. In some instances, blockers have a Tm of about 70, 72, 75, 77, 80, 82, 85, 88, 90, 92, or about 95 degrees C. In some instances, blockers have a Tm of 78 degrees C. to 90 degrees C. In some instances, blockers have a Tm of 79 degrees C. to 90 degrees C. In some instances, blockers have a Tm of 80 degrees C. to 90 degrees C. In some instances, blockers have a Tm of 81 degrees C. to 90 degrees C. In some instances, blockers have a Tm of 82 degrees C. to 90 degrees C. In some instances, blockers have a Tm of 83 degrees C. to 90 degrees C. In some instances, blockers have a Tm of 84 degrees C. to 90 degrees C. In some instances, a set of blockers have an average Tm of 78 degrees C. to 90 degrees C. In some instances, a set of blockers have an average Tm of 80 degrees C. to 90 degrees C. In some instances, a set of blockers have an average Tm of at least 80 degrees C. In some instances, a set of blockers have an average Tm of at least 81 degrees C. In some instances, a set of blockers have an average Tm of at least 82 degrees C. In some instances, a set of blockers have an average Tm of at least 83 degrees C. In some instances, a set of blockers have an average Tm of at least 84 degrees C. In some instances, a set of blockers have an average Tm of at least 86 degrees C. Blocker Tm are in some instances modified as a result of other components described herein, such as use of a fast hybridization buffer and/or hybridization enhancer.

The molar ratio of blockers to adapter targets may influence the off-bait (and subsequently off-target) rates during hybridization. The more efficient a blocker is at binding to the target adapter, the less blocker is required. Blockers described herein in some instances achieve sequencing outcomes of no more than 20% off-target reads with a molar ratio of less than 20:1 (blocker:target). In some instances, no more than 20% off-target reads are achieved with a molar ratio of less than 10:1 (blocker:target). In some instances, no more than 20% off-target reads are achieved with a molar ratio of less than 5:1 (blocker:target). In some instances, no more than 20% off-target reads are achieved with a molar ratio of less than 2:1 (blocker:target). In some instances, no more than 20% off-target reads are achieved with a molar ratio of less than 1.5:1 (blocker:target). In some instances, no more than 20% off-target reads are achieved with a molar ratio of less than 1.2:1 (blocker:target). In some instances, no more than 20% off-target reads are achieved with a molar ratio of less than 1.05:1 (blocker:target).

The universal blockers may be used with panel libraries of varying size. In some embodiments, the panel libraries comprises at least or about 0.01, 0.02, 0.03, 0.04, 0.05, 0.06, 0.07, 0.08, 0.09, 1.0, 2.0, 4.0, 8.0, 10.0, 12.0, 14.0, 16.0, 18.0, 20.0, 22.0, 24.0, 26.0, 28.0, 30.0, 40.0, 50.0, 60.0, or more than 60.0 megabases (Mb).

Blockers as described herein may improve on-target performance. In some embodiments, on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% for various index designs. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% is improved for various panel sizes.

### De Novo Synthesis of Small Polynucleotide Populations for Amplification Reactions

Described herein are methods of synthesis of polynucleotides from a surface, e.g., a plate. In some instances, polynucleotide libraries comprise sample polynucleotide libraries. In some instances, the polynucleotides are synthesized on a cluster of loci for polynucleotide extension, released and then subsequently subjected to an amplification reaction, e.g., PCR. A silicon plate in some instances includes multiple clusters. Within each cluster are multiple loci. Polynucleotides are synthesized de novo on a plate from the cluster. Polynucleotides are cleaved and removed from the plate to form a population of released polynucleotides. The population of released polynucleotides is then amplified to form a library of amplified polynucleotides.

Provided herein are methods where amplification of polynucleotides synthesized on a cluster provide for enhanced control over polynucleotide representation compared to amplification of polynucleotides across an entire surface of a structure without such a clustered arrangement. In some instances, amplification of polynucleotides synthesized from a surface having a clustered arrangement of loci for polynucleotides extension provides for overcoming the negative effects on representation due to repeated synthesis of large polynucleotide populations. Exemplary negative effects on representation due to repeated synthesis of large polynucleotide populations include, without limitation, amplification bias resulting from high/low GC content, repeating sequences, trailing adenines, secondary structure, affinity for target sequence binding, or modified nucleotides in the polynucleotide sequence.

Cluster amplification as opposed to amplification of polynucleotides across an entire plate without a clustered arrangement can result in a tighter distribution around the mean. For example, if 100,000 reads are randomly sampled, an average of 8 reads per sequence would yield a library with a distribution of about 1.5× from the mean. In some cases, single cluster amplification results in at most about 1.5×, 1.6×, 1.7×, 1.8×, 1.9×, or 2.0× from the mean. In some cases, single cluster amplification results in at least about 1.0×, 1.2×, 1.3×, 1.5× 1.6×, 1.7×, 1.8×, 1.9×, or 2.0× from the mean.

Cluster amplification methods described herein when compared to amplification across a plate can result in a polynucleotide library that requires less sequencing for equivalent sequence representation. In some instances at least 10%, at least 20%, at least 30%, at least 40%, at least 50%, at least 60%, at least 70%, at least 80%, at least 90%, or at least 95% less sequencing is required. In some instances up to 10%, up to 20%, up to 30%, up to 40%, up to 50%, up to 60%, up to 70%, up to 80%, up to 90%, or up to 95% less sequencing is required. Sometimes 30% less sequencing is required following cluster amplification compared to amplification across a plate. Sequencing of polynucleotides in some instances is verified by high-throughput sequencing such as by next generation sequencing. Sequencing of the sequencing library can be performed with any appropriate sequencing technology, including but not limited to single-molecule real-time (SMRT) sequencing, polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. The number of times a single nucleotide or polynucleotide is identified or “read” is defined as the sequencing depth or read depth. In some cases, the read depth is referred to as a fold coverage, for example, 55 fold (or 55×) coverage, optionally describing a percentage of bases.

In some instances, amplification from a clustered arrangement compared to amplification across a plate results in less dropouts, or sequences which are not detected after sequencing of amplification product. Dropouts can be of AT and/or GC. In some instances, a number of dropouts are at most about 1%, 2%, 3%, 4%, or 5% of a polynucleotide population. In some cases, the number of dropouts is zero.

A cluster as described herein comprises a collection of discrete, non-overlapping loci for polynucleotide synthesis. A cluster can comprise about 50-1000, 75-900, 100-800, 125-700, 150-600, 200-500, or 300-400 loci. In some instances, each cluster includes 121 loci. In some instances, each cluster includes about 50-500, 50-200, 100-150 loci. In some instances, each cluster includes at least about 50, 100, 150, 200, 500, 1000 or more loci. In some instances, a single plate includes 100, 500, 10000, 20000, 30000, 50000, 100000, 500000, 700000, 1000000 or more loci. A locus can be a spot, well, microwell, channel, or post. In some instances, each cluster has at least 1×, 2×, 3×, 4×, 5×, 6×, 7×, 8×, 9×, 10×, or more redundancy of separate features supporting extension of polynucleotides having identical sequence.

Generation of Polynucleotide Libraries with Controlled Stoichiometry of Sequence Content

In some instances, the polynucleotide library (such as a sample polynucleotide set for variant detection) is synthesized with a specified distribution of desired polynucleotide sequences. In some instances, adjusting polynucleotide libraries for enrichment of specific desired sequences results in improved downstream application outcomes.

One or more specific sequences can be selected based on their evaluation in a downstream application. In some instances, the evaluation is binding affinity to target sequences for amplification, enrichment, or detection, stability, melting temperature, biological activity, ability to assemble into larger fragments, or other property of polynucleotides. In some instances, the evaluation is empirical or predicted from prior experiments and/or computer algorithms. An exemplary application includes increasing sequences in a probe library which correspond to areas of a genomic target having less than average read depth.

Selected sequences in a polynucleotide library can be at least 10%, 20%, 30%, 40%, 50%, 60%, 70%, 80%, 90%, 95%, or more than 95% of the sequences. In some instances, selected sequences in a polynucleotide library are at most 10%, 20%, 30%, 40%, 50%, 60%, 70%, 80%, 90%, 95%, or at most 100% of the sequences. In some cases, selected sequences are in a range of about 5-95%, 10-90%, 30-80%, 40-75%, or 50-70% of the sequences.

Polynucleotide libraries can be adjusted for the frequency of each selected sequence. In some instances, polynucleotide libraries favor a higher number of selected sequences. For example, a library is designed where increased polynucleotide frequency of selected sequences is in a range of about 40% to about 90%. In some instances, polynucleotide libraries contain a low number of selected sequences. For example, a library is designed where increased polynucleotide frequency of the selected sequences is in a range of about 10% to about 60%. A library can be designed to favor a higher and lower frequency of selected sequences. In some instances, a library favors uniform sequence representation. For example, polynucleotide frequency is uniform with regard to selected sequence frequency, in a range of about 10% to about 90%. In some instances, a library comprises polynucleotides with a selected sequence frequency of about 10% to about 95% of the sequences.

Generation of polynucleotide libraries with a specified selected sequence frequency in some cases occurs by combining at least 2 polynucleotide libraries with different selected sequence frequency content. In some instances, at least 2, 3, 4, 5, 6, 7, 10, or more than 10 polynucleotide libraries are combined to generate a population of polynucleotides with a specified selected sequence frequency. In some cases, no more than 2, 3, 4, 5, 6, 7, or 10 polynucleotide libraries are combined to generate a population of non-identical polynucleotides with a specified selected sequence frequency.

In some instances, selected sequence frequency is adjusted by synthesizing fewer or more polynucleotides per cluster. For example, at least 25, 50, 100, 200, 300, 400, 500, 600, 700, 800, 900, 1000, or more than 1000 non-identical polynucleotides are synthesized on a single cluster. In some cases, no more than about 50, 100, 200, 300, 400, 500, 600, 700, 800, 900, 1000 non-identical polynucleotides are synthesized on a single cluster. In some instances, 50 to 500 non-identical polynucleotides are synthesized on a single cluster. In some instances, 100 to 200 non-identical polynucleotides are synthesized on a single cluster. In some instances, about 100, about 120, about 125, about 130, about 150, about 175, or about 200 non-identical polynucleotides are synthesized on a single cluster.

In some cases, selected sequence frequency is adjusted by synthesizing non-identical polynucleotides of varying length. For example, the length of each of the non-identical polynucleotides synthesized may be at least or about at least 10, 15, 20, 25, 30, 35, 40, 45, 50, 100, 150, 200, 300, 400, 500, 2000 nucleotides, or more. The length of the non-identical polynucleotides synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the non-identical polynucleotides synthesized may fall from 10-2000, 10-500, 9-400, 11-300, 12-200, 13-150, 14-100, 15-50, 16-45, 17-40, 18-35, and 19-25.

### Kits

Provided herein are kits comprising libraries of polynucleotides. In some instances, a kit comprises one or more of a reference standards (controls), wherein the reference standard comprises a sample polynucleotide set and a background set; instructions for use of the kit contents; and packaging to hold and describe the kit contents. In some instances, a kit comprises at least two standards selected from sample polynucleotides having a VAF of 0%, 0.1% 0.25%, 0.5%, 1%, or 2% relative to a wild-type genomic sequence. In some instances, a kit comprises five standards each having a VAF of 0%, 0.1% 0.25%, 0.5%, 1%, or 2% relative to a wild-type genomic sequence. In some instances, kits comprise instructions of use of reference standards with one or more sequencing instruments or other instrument which is configured to measure genomic variants. In some instances, the reference standard is packaged in a buffer. In some instances, the reference standard is packaged in a tube. In some instances, the reference standard is not packaged in a plasma-like format. In some instances, the reference standard comprises 500 ng to 5 micrograms of total DNA.

### Next Generation Sequencing

Downstream applications of polynucleotide libraries (such as sample polynucleotide sets or reference standards) may include next generation sequencing. For example, enrichment of target sequences with a controlled stoichiometry polynucleotide probe library results in more efficient sequencing. The performance of a polynucleotide library for capturing or hybridizing to targets may be defined by a number of different metrics describing efficiency, accuracy, and precision. For example, Picard metrics comprise variables such as HS library size (the number of unique molecules in the library that correspond to target regions, calculated from read pairs), mean target coverage (the percentage of bases reaching a specific coverage level), depth of coverage (number of reads including a given nucleotide) fold enrichment (sequence reads mapping uniquely to the target/reads mapping to the total sample, multiplied by the total sample length/target length), percent off-bait bases (percent of bases not corresponding to bases of the probes/baits), percent off-target (percent of bases not corresponding to bases of interest), usable bases on target, AT or GC dropout rate, fold 80 base penalty (fold over-coverage needed to raise 80 percent of non-zero targets to the mean coverage level), percent zero coverage targets, PF reads (the number of reads passing a quality filter), percent selected bases (the sum of on-bait bases and near-bait bases divided by the total aligned bases), percent duplication, or other variable consistent with the specification.

Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of % coverage (or coverage breadth). For example, 10 million reads of a 1 million base genome, perfectly distributed, theoretically results in 10× read depth of 100% of the sequences. In practice, a greater number of reads (higher theoretical read depth, or oversampling) may be needed to obtain the desired read depth for a percentage of the target sequences. Enrichment of target sequences with a controlled stoichiometry probe library increases the efficiency of downstream sequencing, as fewer total reads will be required to obtain an outcome with an acceptable number of reads over a desired percentage (%) of target sequences. For example, in some instances 55× theoretical read depth of target sequences results in at least 30× coverage of at least 90% of the sequences. In some instances no more than 55× theoretical read depth of target sequences results in at least 30× read depth of at least 80% of the sequences. In some instances no more than 55× theoretical read depth of target sequences results in at least 30× read depth of at least 95% of the sequences. In some instances no more than 55× theoretical read depth of target sequences results in at least 10× read depth of at least 98% of the sequences. In some instances, 55× theoretical read depth of target sequences results in at least 20× read depth of at least 98% of the sequences. In some instances no more than 55× theoretical read depth of target sequences results in at least 5× read depth of at least 98% of the sequences. Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5χ. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth. In some instances, sequencing is performed to achieve a theoretical read depth of at least 30×, 50×, 100×, 150×, 200×, 250×, 300×, 500×, or at least 1000×. In some instances, sequencing is performed to achieve a theoretical read depth of about 30×, 50×, 100×, 150×, 200×, 250×, 300×, 500×, or about 1000×. In some instances, sequencing is performed to achieve a theoretical read depth of no more than 30×, 50×, 100×, 150×, 200×, 250×, 300×, 500×, or no more than 1000×. In some instances, sequencing is performed to achieve an actual read depth of at least 30×, 50×, 100×, 150×, 200×, 250×, 300×, 500×, or at least 1000×. In some instances, sequencing is performed to achieve an actual read depth of no more than 30×, 50×, 100×, 150×, 200×, 250×, 300×, 500×, or no more than 1000×. In some instances, sequencing is performed to achieve an actual read depth of about 30×, 50×, 100×, 150×, 200×, 250×, 300×, 500×, or about 1000×.

On-target rate represents the percentage of sequencing reads that correspond with the desired target sequences. In some instances, a controlled stoichiometry polynucleotide probe library results in an on-target rate of at least 30%, or at least 35%, 40%, 45%, 50%, 55%, 60%, 65%, 70%, 75%, 80%, 85%, or at least 90%. Increasing the concentration of polynucleotide probes during contact with target nucleic acids leads to an increase in the on-target rate. In some instances, the concentration of probes is increased by at least 1.5×, 2.0×, 2.5×, 3×, 3.5×, 4×, 5×, or more than 5×. In some instances, increasing the probe concentration results in at least a 20% increase, or a 10%, 20%, 30%, 40%, 50%, 60%, 70%, 80%, 90%, 100%, 200%, 300%, or at least a 500% increase in on-target binding. In some instances, increasing the probe concentration by 3× results in a 20% increase in on-target rate.

Coverage uniformity is in some cases calculated as the read depth as a function of the target sequence identity. Higher coverage uniformity results in a lower number of sequencing reads needed to obtain the desired read depth. For example, a property of the target sequence may affect the read depth, for example, high or low GC or AT content, repeating sequences, trailing adenines, secondary structure, affinity for target sequence binding (for amplification, enrichment, or detection), stability, melting temperature, biological activity, ability to assemble into larger fragments, sequences containing modified nucleotides or nucleotide analogues, or any other property of polynucleotides. Enrichment of target sequences with controlled stoichiometry polynucleotide probe libraries results in higher coverage uniformity after sequencing. In some instances, 95% of the sequences have a read depth that is within 1× of the mean library read depth, or about 0.05, 0.1, 0.2, 0.5, 0.7, 1, 1.2, 1.5, 1.7 or about within 2× the mean library read depth. In some instances, 80%, 85%, 90%, 95%, 97%, or 99% of the sequences have a read depth that is within 1× of the mean.

Enrichment of Target Nucleic Acid Molecules with a Polynucleotide Probe Library

A probe library described herein may be used to enrich target polynucleotides present in a population of sample polynucleotides, for a variety of downstream applications. In one some instances, a sample is obtained from one or more sources, and the population of sample polynucleotides is isolated. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as T4 DNA polymerase, klenow enzyme, and T4 polynucleotide kinase in an appropriate buffer. A nucleotide overhang to facilitate ligation to adapters is added, in some instances with 3′ to 5′ exo minus klenow fragment and dATP.

Adapters (such as universal adapters) may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flowcell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters. A library of double stranded adapter-tagged polynucleotide strands is contacted with polynucleotide probes, to form hybrid pairs. Such pairs are separated from un-hybridized fragments, and isolated from probes to produce an enriched library. The enriched library may then be sequenced.

The library of double stranded sample nucleic acid fragments is then denatured in the presence of adapter blockers. Adapter blockers minimize off-target hybridization of probes to the adapter sequences (instead of target sequences) present on the adapter-tagged polynucleotide strands, and/or prevent intermolecular hybridization of adapters (e.g., “daisy chaining”). Denaturation is carried out in some instances at 96° C., or at about 85, 87, 90, 92, 95, 97, 98 or about 99° C. A polynucleotide targeting library (probe library) is denatured in a hybridization solution, in some instances at 96° C., at about 85, 87, 90, 92, 95, 97, 98 or 99° C. The denatured adapter-tagged polynucleotide library and the hybridization solution are incubated for a suitable amount of time and at a suitable temperature to allow the probes to hybridize with their complementary target sequences. In some instances, a suitable hybridization temperature is about 45 to 80° C., or at least 45, 50, 55, 60, 65, 70, 75, 80, 85, or 90° C. In some instances, the hybridization temperature is 70° C. In some instances, a suitable hybridization time is 16 hours, or at least 4, 6, 8, 10, 12, 14, 16, 18, 20, 22, or more than 22 hours, or about 12 to 20 hours. Binding buffer is then added to the hybridized adapter-tagged-polynucleotide probes, and a solid support comprising a capture moiety is used to selectively bind the hybridized adapter-tagged polynucleotide-probes. The solid support is washed with buffer to remove unbound polynucleotides before an elution buffer is added to release the enriched, tagged polynucleotide fragments from the solid support. In some instances, the solid support is washed 2 times, or 1, 2, 3, 4, 5, or 6 times. The enriched library of adapter-tagged polynucleotide fragments is amplified and the enriched library is sequenced.

A plurality of nucleic acids (e.g. genomic sequence) may be obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio nanopore sequencing, or BGI/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, e.g., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing. Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles et al., “Genome sequencing in microfabricated high-density picolitre reactors”, Nature, 2005, Vol. 437, Pages 376-380.

In some instances, high-throughput sequencing is performed using Clonal Single Molecule Array (Solexa, Inc.) or sequencing-by-synthesis (SBS) utilizing reversible terminator chemistry. See, e.g., Constans, A., The Scientist, 2003, Vol. 17, Issue 13, Page 36. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Overall such systems involve sequencing a target oligonucleotide molecule having a plurality of bases by the temporal addition of bases via a polymerization reaction that is measured on a molecule of oligonucleotide, i e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next generation sequencing technique can comprise real-time (SMRT™) technology by Pacific Biosciences. In SMRT, each of four DNA bases can be attached to one of four different fluorescent dyes. These dyes can be phospho-linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing. See, e.g., Soni et al., Clin Chem., 2007, Vol. 53, Pages 1996-2001. A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SiO2). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors. See e.g., Garaj et al., Nature, 2010, Vol. 467, Pages 190-193. A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing. See, e.g., by Complete Genomics; and Drmanac et al., Science, 2010, Vol. 327, Pages 78-81. DNA can be isolated, fragmented, and size selected. For example, DNA can be fragmented (e.g., by sonication) to a mean length of about 500 bp. Adaptors (Ad1) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Ad1 adapter. A restriction enzyme (e.g., Acul) can be applied, and the DNA can be cleaved 13 bp to the left of the Ad1 to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

A population of polynucleotides may be enriched prior to adapter ligation. In one example, a plurality of polynucleotides is obtained from a sample, fragmented, optionally end-repaired, and denatured at high temperature, preferably 90-99° C. A polynucleotide targeting library (probe library) is denatured in a hybridization solution at high temperature, preferably about 90 to 99 C, and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched polynucleotide fragments are then polyadenylated, adapters are ligated to both ends of the polynucleotide fragments to produce a library of adapter-tagged polynucleotide strands, and the adapter-tagged polynucleotide library is amplified. The adapter-tagged polynucleotide library is then sequenced.

A polynucleotide targeting library may also be used to filter undesired sequences from a plurality of polynucleotides, by hybridizing to undesired fragments. For example, a plurality of polynucleotides is obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. Alternatively, adenylation and adapter ligation steps are instead performed after enrichment of the sample polynucleotides. The adapter-tagged polynucleotide library is then denatured at high temperature, preferably 90-99° C., in the presence of adapter blockers. A polynucleotide filtering library (probe library) designed to remove undesired, non-target sequences is denatured in a hybridization solution at high temperature, preferably about 90 to 99° C., and combined with the denatured, tagged polynucleotide library in hybridization solution for about 10 to 24 hours at about 45 to 80 C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 1 and 5 times to elute unbound adapter-tagged polynucleotide fragments. The enriched library of unbound adapter-tagged polynucleotide fragments is amplified and then the amplified library is sequenced.

### Highly Parallel De Novo Nucleic Acid Molecule Synthesis

Described herein is a platform approach utilizing miniaturization, parallelization, and vertical integration of the end-to-end process from polynucleotide synthesis to gene assembly within Nano wells on silicon to create a revolutionary synthesis platform. Devices described herein provide, with the same footprint as a 96-well plate, a silicon synthesis platform is capable of increasing throughput by a factor of 100 to 1,000 compared to traditional synthesis methods, with production of up to approximately 1,000,000 polynucleotides in a single highly-parallelized run. In some instances, a single silicon plate described herein provides for synthesis of about 6,100 non-identical polynucleotides. In some instances, each of the non-identical polynucleotides is located within a cluster. A cluster may comprise 50 to 500 non-identical polynucleotides.

Methods described herein provide for synthesis of a library of polynucleotides each encoding for a predetermined variant of at least one predetermined reference nucleic acid sequence. In some cases, the predetermined reference sequence is nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. The synthesized specific alterations in the nucleic acid sequence can be introduced by incorporating nucleotide changes into overlapping or blunt ended polynucleotide primers. Alternatively, a population of polynucleotides may collectively encode for a long nucleic acid (e.g., a gene) and variants thereof. In this arrangement, the population of polynucleotides can be hybridized and subject to standard molecular biology techniques to form the long nucleic acid (e.g., a gene) and variants thereof. When the long nucleic acid (e.g., a gene) and variants thereof are expressed in cells, a variant protein library is generated. Similarly, provided here are methods for synthesis of variant libraries encoding for RNA sequences (e.g., miRNA, shRNA, and mRNA) or DNA sequences (e.g., enhancer, promoter, UTR, and terminator regions). Also provided here are downstream applications for variants selected out of the libraries synthesized using methods described here. Downstream applications include identification of variant nucleic acid or protein sequences with enhanced biologically relevant functions, e.g., biochemical affinity, enzymatic activity, changes in cellular activity, and for the treatment or prevention of a disease state.

### Substrates

Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, micro well, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some instances, a surface of a device is inclusive of one or a plurality of surfaces of a substrate.

Provided herein are structures that may comprise a surface that supports the synthesis of a plurality of polynucleotides having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence.

### Surface Materials

Provided herein is a device comprising a surface, wherein the surface is modified to support polynucleotide synthesis at predetermined locations and with a resulting low error rate, a low dropout rate, a high yield, and a high oligo representation. In some instances, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (e.g., polytetrafluoroethylene, polypropylene, polystyrene, polycarbonate, and blends thereof, and metals (e.g., gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

### Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a polynucleotide synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

### Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, e.g., providing, modifying or substituting surface functional groups, (3) defunctionalizing a surface, e.g., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

### Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, e.g., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 minutes, 1 minute, 50 seconds, 40 seconds, 30 seconds, 20 seconds and 10 seconds.

### Large Polynucleotide Libraries Having Low Error Rates

Average error rates for polynucleotides synthesized within a library using the systems and methods provided may be less than 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for polynucleotides synthesized within a library using the systems and methods provided are less than 1/1000.

In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/500, 1/600, 1/700, 1/800, 1/900, or 1/1000. In some instances, aggregate error rates for polynucleotides synthesized within a library using the systems and methods provided are less than 1/1000.

In some instances, an error correction enzyme may be used for polynucleotides synthesized within a library using the systems and methods provided can use. In some instances, aggregate error rates for polynucleotides with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for polynucleotides synthesized within a library using the systems and methods provided can be less than 1/500, 1/600, 1/700, 1/800, 1/900, or 1/1000. In some instances, aggregate error rates with error correction for polynucleotides synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from polynucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large polynucleotide and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic polynucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

The present disclosure is further described by the following non-limiting items.

Item 1. A composition comprising:
(a) a first polynucleotide adapter comprising:
   a first strand, wherein the first strand comprises a first terminal adapter region, a first non-complementary region, and a first yoke region;
   a second strand, wherein the second strand comprises a second terminal adapter region, a second non-complementary region, and a second yoke region;
   wherein the first yoke region and the second yoke region are complementary, wherein the first non-complementary region and the second non-complementary region are not complementary,
(b) a second polynucleotide adapter comprising:
   a third strand, wherein the third strand comprises a third terminal adapter region, a third non-complementary region, and a third yoke region; and
   a fourth strand, wherein the fourth strand comprises a fourth terminal adapter region, a fourth non-complementary region, and a fourth yoke region,
   wherein the third yoke region and the fourth yoke region are complementary, and wherein the third non-complementary region and the fourth non-complementary region are not complementary, and
wherein the first polynucleotide adapter and the second polynucleotide adapter are connected via linker.

Item 2. The composition of item 1, wherein the linker is attached to the first terminal adapter region and the third terminal adapter region.

Item 3. The composition of item 1, wherein the linker is attached to the second terminal adapter region and the fourth terminal adapter region.

Item 4. The composition of item 1, wherein the linker connects the 5′ termini of one or more strands.

Item 5. The composition of item 1, wherein the linker connects the 3′ termini of one or more strands.

Item 6. The composition of any one of items 1-3, wherein the linker comprises an alkene, alkyne, triazine, triazole, oxime, sulfide, amide, or cyclooctane.

Item 7. The composition of any one of items 1-4, wherein the linker comprises a PEG unit.

Item 8. The composition of item 7, wherein the linker comprises 5-50 PEG units.

Item 9. The composition of any one of items 1-8, wherein the linker comprises Spacer 18 (Sp18).

Item 10. The composition of any one of items 1-8, wherein the linker comprises 1 to 20 Sp18.

Item 11. The composition of any one of items 1-13, wherein the linker comprises a polynucleotide.

Item 12. The composition of item 11, wherein the linker comprises at least one cleavable base.

Item 13. The composition of item 12, wherein the cleavable base is a uracil base.

Item 14. The composition of item 12, wherein the linker comprises at least one restriction endonuclease site.

Item 15. The composition of item 11, wherein the linker comprises a duplex region.

Item 16. The composition of item 11, wherein the linker comprises at least one splint polynucleotide.

Item 17. The composition of item 16, wherein the at least one splint polynucleotide is at least partially complementary to a portion of the first polynucleotide adapter or the second polynucleotide adapter.

Item 18. The composition of item 17, wherein the linker comprises at least two splint polynucleotides, wherein the at least two splint polynucleotides are at least partially overlapped with each other.

Item 19. The composition of item 16, wherein the linker comprises at least two splint polynucleotides, wherein the at least two splint polynucleotides are at least partially overlapped with a portion of the first polynucleotide adapter or the second polynucleotide adapter.

Item 20. The composition of any one of items 1-19, wherein the first polynucleotide adapter and the second polynucleotide adapter are covalently attached to the linker.

Item 21. The composition of any one of items 1-19, wherein the first polynucleotide adapter and the second polynucleotide adapter are non-covalently attached to the linker.

Item 22. The composition of any one of items 1-21, wherein one or more of the first polynucleotide adapter and the second polynucleotide adapter comprises at least one barcode.

Item 23. The composition of item 15, wherein the at least one barcode comprises one or more of a cell index, sample index, or unique molecular identifier (UMI).

Item 24. The composition of any one of items 1-23, wherein one or more of the first polynucleotide adapter and the second polynucleotide adapter comprises at least one alpha thiophosphate.

Item 25. The composition of any one of items 1-24, wherein the 5′ end of at least one of the strands comprises thymidine.

Item 26. An adapter-ligated sample polynucleotide comprising the composition of any one of items 1-25, wherein the composition further comprises at least one sample polynucleotide.

Item 27. The adapter-ligated sample polynucleotide of item 26, wherein the at least one sample polynucleotide is attached to the 3′ end of the first strand and the 5′ end of the second strand.

Item 28. The adapter-ligated sample polynucleotide of item 26 or 27, wherein the at least one sample polynucleotide is attached to the 3′ end of the third strand and the 5′ end of the fourth strand.

Item 29. The adapter-ligated sample polynucleotide of item 26, wherein the at least one sample polynucleotide is attached to the 3′ end of the first strand and the 3′ end of the second strand.

Item 30. The adapter-ligated sample polynucleotide of item 26 or 29, wherein the at least one sample polynucleotide is attached to the 5′ end of the third strand and the 5′ end of the fourth strand.

Item 31. The adapter-ligated sample polynucleotide of any one of items 26-30, wherein the sample polynucleotides comprise genomic DNA.

Item 32. The adapter-ligated sample polynucleotide of any one of items 26-30, wherein the sample polynucleotides comprise cDNA.

Item 33. A library of polynucleotides comprising a plurality of adapter-ligated sample polynucleotides of any one of items 26-32.

Item 34. A plurality of sequencing libraries, wherein the plurality of sequencing libraries comprises one or more libraries of item 33.

Item 35. The plurality of sequencing libraries of item 34, wherein each library is obtained from a different sample.

Item 36. The library of any one of items 33-35, wherein no more than 70% of the sample polynucleotides are present within one standard deviation of the mean sample polynucleotide amount.

Item 37. The library of any one of items 33-35, wherein no more than 50% of the sample polynucleotides are present within one standard deviation of the mean sample polynucleotide amount.

Item 38. The library of any one of items 33-35, wherein no more than 25% of the sample polynucleotides are present within one standard deviation of the mean sample polynucleotide amount.

Item 39. A method of library preparation, comprising:
(a) providing a plurality of sample polynucleotides; and
(b) ligating at least one composition of any one of items 1-38 to at least one sample polynucleotide.

Item 40. The method of item 39, wherein the plurality of sample polynucleotides comprise genomic DNA (gDNA).

Item 41. The method of item 29, wherein the plurality of sample polynucleotides comprise circular DNA (cDNA).

Item 42. The method of any one of items 39-41, wherein the molar ratio of the at least one composition to plurality of sample polynucleotides is no more than 1:5.

Item 43. The method of any one of items 39-41, wherein the molar ratio of the at least one composition to plurality of sample polynucleotides is no more than 1:2.

Item 44. The method of any one of items 39-41, wherein the molar ratio of the at least one composition to plurality of sample polynucleotides is no more than 1:1.

Item 45. The method of any one of items 39-44, wherein ligating occurs with an efficiency of at least 25%.

Item 46. The method of any one of items 39-44, wherein ligating occurs with an efficiency of at least 50%.

Item 47. The method of any one of items 39-44, wherein ligating occurs with an efficiency of at least 75%.

Item 48. The method of any one of items 39-47, wherein the method further comprises cleaving the linker.

Item 49. The method of any one of items 39-47, wherein cleaving the linker comprises contacting the conjugate with an enzyme.

Item 50. The method of item 49, wherein cleaving the enzyme comprises USER or a site-specific restriction endonuclease.

Item 51. A conjugate comprising:
a first strand, wherein the first strand comprises a first terminal adapter region, a first non-complementary region, and a first yoke region; and
a second strand, wherein the second strand comprises a second terminal adapter region, a second non-complementary region, and a second yoke region,
wherein the first strand and the second strand are connected via linker.

Item 52. The conjugate of item 51, wherein the linker is attached to the 5′ end of the first strand and the 5′ end of the second strand.

Item 53. The conjugate of item 51, wherein the linker is attached to the 3′ end of the first strand and the 3′ end of the second strand.

Item 54. A method of generating a composition of any one of items 1-25 or a conjugate of any one of items 51-53 comprising:
contacting the first strand with the second strand, wherein the first strand comprises a first reactive group and the second strand comprises a second reactive group, wherein reaction of the first reactive group and the second reactive group generates the conjugate.

Item 55. The method of item 54, wherein the first strand further comprises a first linker.

Item 56. The method of item 54 or 55, wherein the second strand further comprises a second linker.

Item 57. The method of item 55 or 56, wherein the first linker comprises the first reactive group.

Item 58. The method of item 56 or 57, wherein the second linker comprises the second reactive group.

Item 59. A method of generating a composition of any one of items 1-25 or a conjugate of any one of items 51-53 comprising:
(a) coupling a nucleotide monomer to a growing chain on a surface;
(b) repeating step (a) to generate the first strand of any one of items 1-25;
(c) coupling a linker comprising a reactive group to a terminus of the first strand; and
(d) repeating step (a) to generate the third strand of any one of items 1-25.

Item 60. The method of item 59, wherein the method further comprises cleaving the composition from the solid support.

Item 61. The method of item 59 or 60, wherein the method comprises hybridizing the second strand to the first strand.

Item 62. The method of any one of items 59-61, wherein the method comprises hybridizing the fourth strand to the third strand.

### EXAMPLES

The following examples are given for the purpose of illustrating various embodiments of the present disclosure and are not intended to be limiting in any way.

### Example 1: Synthesis of Hybrid Circular Adapters

This example demonstrates the synthesis of exemplary adapter conjugates as described herein.

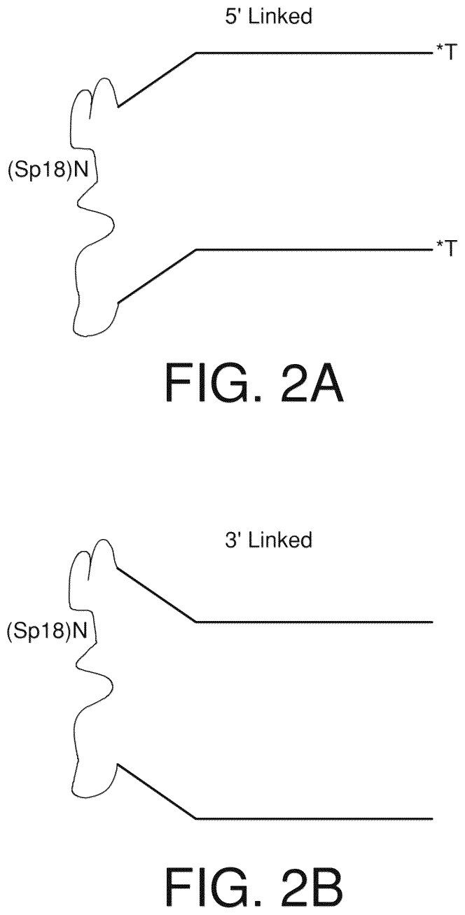

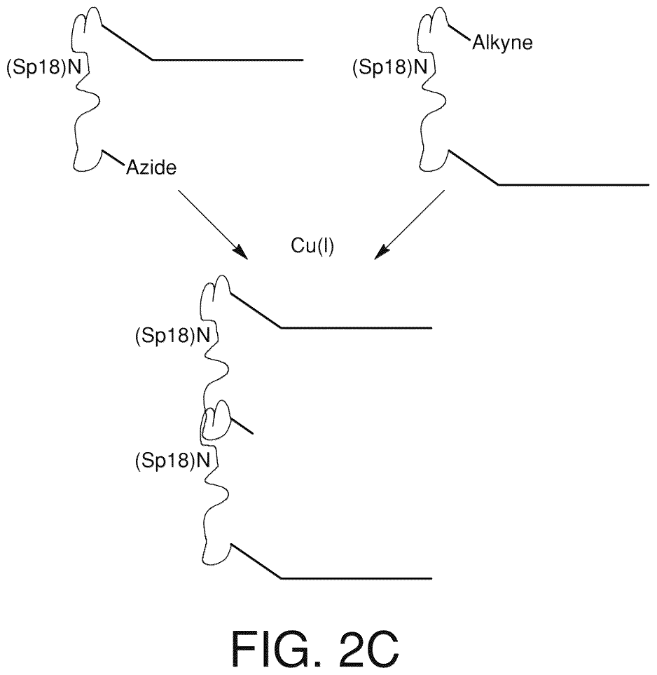

As illustrated in FIGS. 2A-2C, adapter strands were synthesized by click reaction of two stubby-Y adapters (“stub adapters”) having the sequences shown in Table 2, below. Both 500 stub adapters were allowed to react in the presence of a Cu(I) molecule catalyzed to form an exemplary 5′-linked adapter dimer.

TABLE 2
Stubby-Y Adapter Sequences

| Stub Name | Polynucleotide Sequence (5′-3′) |
|---|---|
| 500-STUB- | /5AzideN/AAAAA (SEQ ID NO: 35)/ |
| AZ_Sp18-v1 | I-Sp18/ACACTCTTTCCCTACACGACGCT |
| | CTTCCGATC*T (SEQ ID NO: 36) |
| 500-STUB- | /5Hexynl/AAAAA (SEQ ID NO: 35) |
| ALK_Sp18-v1 | /I-Sp18/ |
| | ACACTCTTTCCCTACACGACGCTCTTCCG |
| | ATC*T (SEQ ID NO: 36) |

*indicates a phosphorothioate bond.
“I-Sp18” indicates Spacer 18 (PEG) as an internal modification.

In another embodiment, adapter strands were directly synthesized using solid-phase polynucleotide synthesis. Phosphoramidites were added to generate adapter polynucleotide sequences, followed by reaction with a linker. Phosphoramidite synthesis was then continued using reverse amidites in the 5′ to 3′ direction to complete the sequence. The adapter-linker-adapter conjugates were cleaved from the surface and hybridized with 500 stub adapter and 700 stub adapter shown in Table 3A, below, to generate compositions having the sequences shown in Table 3B, below.

TABLE 3A
Stubby-Y Adapter Sequences

| Stub Name | Polynucleotide Sequence (5′-3′) |
|---|---|
| 500-STUB-v1 | ACACTCTTTCCCTACACGACGCTCTT |
| | CCGATC*T (SEQ ID NO: 36) |
| 700-STUB-v1 | /5Phos/GATCGGAAGAGCACACGTC |
| | TGAACTCCAGTCA* C (SEQ ID NO: 37) |

*indicates a phosphorothioate bond.
“5Phos” indicates phosphorylation as a 5′ end modification.

TABLE 3B
Hybrid Circular Adapter Sequences

| Adapter | |
|---|---|
| Name | Polynucleotide Sequence |
| 500-5- | 3′-T*CTAGCCTTCTCGCAGCACAT |
| linked- | CCCTTTCTCACA-5′-/Sp18/-5′- |
| 1sp18-v1 | ACACTCTTTCCCTACACGACGCTCT |
| | TCCGATC*T-3′ |
| | (SEQ ID NO: 36 in the 3′-5′ |
| | direction)-/Sp18/- |
| | (SEQ ID NO: 36 in the 5′-3′ |
| | direction) |
| 500-5- | 3′-T*CTAGCCTTCTCGCAGCACATC |
| linked- | CCTTTCTCACA-5′-/Sp18//Sp18/- |
| 2sp18-v1 | 5′-ACACTCTTTCCCTACACGACGCTCT |
| | TCCGATC*T-3′ |
| | (SEQ ID NO: 36 in the 3′-5′ |
| | direction)/Sp18//Sp18/(SEQ ID |
| | NO: 36 in the 5′-3′ direction) |
| 700-3- | 5′-/5Phos/G*ATCGGAAGAGCACACGT |
| linked- | CTGAACTCCAGTCAC-3′/Sp18/3′- |
| 1sp18-v1 | CACTGACCTCAAGTCTGCACACGAGAAG |
| | GCTA* G/5Phos/-5′ |
| | /5Phos/(SEQ ID NO: 38 in the |
| | 5′-3′ direction)/Sp18/ |
| | (SEQ ID NO: 38 in the 3′-5′ |
| | direction)/5Phos/ |
| 700-3- | 5′-/5Phos/G*ATCGGAAGAGCACAC |
| linked- | GTCTGAACTCCAGTCAC-3′-/Sp18/ |
| 2sp18-v1 | /Sp18/-3′-CACTGACCTCAAGTCT |
| | GCACACGAGAAGGCTA* |
| | G/5Phos/-5′ |
| | /5Phos/(SEQ ID NO: 38 in the |
| | 5′-3′ direction)/Sp18//Sp18/ |
| | (SEQ ID NO: 38 in the 5′-3′ |
| | direction)/5Phos/ |

*indicates a phosphorothioate bond.
“Sp18” indicates Spacer 18 (PEG).
“5Phos” indicates phosphorylation as a 5′ end modification.

### Example 2: High-Throughput Sample Balancing with Hybrid Circular Adapters

This example demonstrates the ability of exemplary adapter conjugates (hybrid circular adapters) as described herein to normalize sample amounts of gDNA during high-throughput sequencing to improve efficiency without decreasing accuracy.

gDNA libraries multiplexed on a sequencer in some instances benefit from balancing sequencing load evenly across all samples in terms of number of molecules (i.e., some samples have very high concentration of gDNA, other samples have much lower concentrations of gDNA). Generally, a large excess of adapters is used for library construction. Since two independent ligation products per molecule are used, conversion cannot be easily controlled by modifying adapter concentration. In high-throughput settings in some instances this leads to challenges where qPCR/dilution is performed per sample. Unbalanced sample loading may also decrease the accuracy of sequencing results. Existing methods to balance samples generally use substantial modification to a library preparation workflow for A-tailing (Normalase), or are limited to transposase based approaches that limit conversions (Nextera Flex, SeqWell).

Hybrid circular adapters are generally compatible with standard sequencing workflows, without substantial modification to the workflow. By linking two adapters, two independent ligation events are replaced with a slower intermolecular ligation and a fast intramolecular ligation. Moreover, steric interactions may be reduced by a flexible linker. Unlike standard adapters, ligation conversion may be controlled by the number of adapter molecules to normalize sample amounts.

Hybrid circular adapters are generated using the general methods of Example 1. In a high-throughput format, 1024 fragmented gDNA samples are ligated to an equal concentration of hybrid-circular adapters adjusted for the minimum sample concentration to generate adapter-ligated polynucleotide libraries for each sample. Samples are then optionally enriched (e.g., exome enrichment), amplified, and subjected to next generation sequencing. The extent of signal normalization (i.e., counts) is measured.

### Example 3: Hybrid Circular Adapters with Cleavable Bases

This example demonstrates the synthesis of exemplary adapter conjugates (hybrid circular adapters) having cleavable bases as described herein and their ability to normalize sample amounts of gDNA during high-throughput sequencing to improve efficiency without decreasing accuracy.

The general methods of Example 1 are used to generate a modified adapter having cleavable bases and the sequences shown in Table 4, below.

TABLE 4
Modified Adapter Sequences

| Adapter Name | Polynucleotide Sequence |
|---|---|
| HCL_2spacers_ | 3′-T*CTAGCCTTCTCGCAGC |
| single_Uracil | ACATCCCTTTCTCACAU-Sp18- |
| | Sp18-5′-5′-ACACTCTTTCC |
| | CTACACGACGCTCTTCCGATC* |
| | T-3′ |
| | (SEQ ID NO: 39 in 3′-5′ |
| | direction)-Sp18-Sp18- |
| | (SEQ ID NO: 36 in 5′- |
| | 3′ direction) |
| HCI_2spacers_ | 3′-T*CTAGCCTTCTCGCAGCA |
| double_Uracil | CATCCCTTTCTCACAUU-Sp18- |
| | Sp18-5′-5′-ACACTCTTTCC |
| | CTACACGACGCTCTTCCGATC* |
| | T-3′ |
| | (SEQ ID NO: 40 in 3′-5′ |
| | direction)-Sp18-Sp18- |
| | (SEQ ID NO: 36 in 5′- |
| | 3′ direction) |
| HCL_3′- | 5′-/5Phos/GATCGGAAGAGCA |
| 3′_linkage_ | CACGTCTGAACTCCAGTCA*C- |
| single_Uracil | Sp18-Sp18-3′-3′- |
| | UC*ACTGACCTCAAGTCTGCACA |
| | CGAGAAGGCTAG/5Phos/-5′ |
| | /5Phos/(SEQ ID NO: 37 |
| | in 5′-3′ direction)- |
| | Sp18-Sp18-(SEQ ID NO: |
| | 41 in the 3′-5′ |
| | direction)/5Phos/ |

*indicates phosphorothioate bond.
“Sp18” indicates Spacer 18.
“5Phos” indicates phosphorylation as a 5′ end modification.
Underlining indicates a cleavable base.

The general methods of Example 2 are used to then test these adapters for sample normalization at 100 mM concentrations.

### Example 4: Hybrid Circular Adapters with Duplex Linkages

This example demonstrates the synthesis of exemplary adapter conjugates (hybrid circular adapters) having temperature-sensitive duplex linkages as described herein and their ability to normalize sample amounts of gDNA during high-throughput sequencing to improve efficiency without decreasing accuracy.

The general methods of Example 1 are used to generate a modified adapter having duplex linkages and the sequences shown in Table 5, below.

TABLE 5
Modified Adapter Sequences

| Adapter | Polynucleotide | |
|---|---|---|
| Name | Sequence (5′-3′) | Length |
| HCL_Over | /5-SpC3/TCGTCGCAATGG | 85 nt |
| hang_Ver | TCTCACGAGGGCACGTAC | |
| A_Y | (SEQ ID NO: 42)/ | |
| | I-Sp18/CCAGGTATCGTGT | |
| | AAGTAGCGA (SEQ ID | |
| | NO: 43)/I-Sp18/ | |
| | ACACTCTTTCCCTACACGAC | |
| | GCTCTTCCGATC*T | |
| | (SEQ ID NO: 36) | |
| | /5-SpC3/TCGTCGCAATGG | |
| | TCTCACGAGGGCACGTAC | |
| | (SEQ ID NO: 42)/ | |
| | I-Sp18/CCAGGTATCGTGT | |
| | AAGTAGCGA (SEQ ID | |
| | NO: 43)/I-Sp18/ | |
| | ACACTCTTTCCCTACACGA | |
| | CGCTCTTCCGATC*T | |
| | (SEQ ID NO: 36) | |
| HCL_Over | /5-SpC3/GTACGTGCCCT | 70 nt |
| hang_Ver | CGTGAGACCATTGCGACGA | |
| A_duplex1 | (SEQ ID NO: 44)/ | |
| | I-Sp18/TCACAGCTGTG | |
| | GATTGGATTGCCCTCCGA | |
| | GTGAGCGTGGC | |
| | (SEQ ID NO: 45)/ | |
| | 3-SpC3/ | |
| HCL_Over | /5-SpC3/GTACGTGCCC | 70 nt |
| hang_Ver | TCGTGAGACCATTGCGAC | |
| A_duplex2 | GA (SEQ ID NO: 44)/ | |
| | I-Sp18/GCCACGCTCAC | |
| | TCGGAGGGCAATCCAATC | |
| | CACAGCTGTGA | |
| | (SEQ ID NO: 46)/ | |
| | 3-SpC3/ | |

*indicates phosphorothioate bond.
“I-Sp18” indicates Spacer 18 (PEG) as an internal modification.
“5-SpC3” indicates Spacer 3 (C3) as a 5′ end modification.
“3-SpC3” indicates Spacer 3 (C3) as a 3′ end modification.

The general methods of Example 2 are used to then test these adapters for sample normalization at 100 mM concentrations.

### Example 5: Hybrid Circular Adapters with Overhang Linkages

This example demonstrates the synthesis of exemplary adapter conjugates (hybrid circular adapters) having temperature-sensitive overhang linkages, as described herein, and their ability to normalize sample amounts of gDNA during high-throughput sequencing to improve efficiency without decreasing accuracy.

The general methods of Example 1 are used to generate a modified adapter having duplex linkages and the sequences shown in Table 6, below.

TABLE 6
Modified Adapter Sequences

| Adapter | | |
|---|---|---|
| Name | Sequence (5′-3′) | Length |
| HCL_Overhang_ | /5-SpC3/GTACGTGCCCT | 85 nt |
| VerB_Left | CGTGAGACCATTGCGACGA | |
| | (SEQ ID NO: 44)/ | |
| | I-Sp18/CCAGGTATCGT | |
| | GTAAGTAGCGA | |
| | (SEQ ID NO: 43)/ | |
| | I-Sp18/ACACTCTTTCC | |
| | CTACACGACGCTCTTCCG | |
| | ATC*T (SEQ ID | |
| | NO: 36) | |
| HCL_Overhang_ | /5-SpC3/TCGTCGCAATGG | 85 nt |
| VerB_Right | TCTCACGAGGGCACGTAC | |
| | (SEQ ID NO: 42)/ | |
| | I-Sp18/CCAGGTATCGTG | |
| | TAAGTAGCGA | |
| | (SEQ ID NO: 43)/ | |
| | I-Sp18/ACACTCTTTCCC | |
| | TACACGACGCTCTTCCGAT | |
| | C*T (SEQ ID | |
| | NO: 36) | |

*indicates phosphorothioate bond.
“I-Sp18” indicates Spacer 18 (PEG) as an internal modification.
“5-SpC3” indicates Spacer 3 (C3) as a 5′ end modification.

The general methods of Example 2 are used to then test these adapters for sample normalization at 100 mM concentrations.

### Example 6: Normalization Test with Hybrid Circular Adapters

This example demonstrates the ability of exemplary adapter conjugates (hybrid circular adapters) to be used to accurately sequence gDNA samples regardless of varying masses of the samples.

Human gDNA molecules were fragmented via mechanical shearing (COVARIS® platform) to yield DNA molecule fragments with an average size of about 200 base pairs. The DNA fragments were aliquoted into various masses ranging from 1 ng to 200 ng for fragment end repair and dA-tailing (i.e., addition of deoxyadenosine monophosphate (dAMP) nucleotides to 3′ end of DNA fragments).

Hybrid circular adapters were generated according to methods described herein (e.g., in Example 1, above) and ligated to DNA fragment molecules in gDNA samples. Reactions were cleaned up and DNA fragment molecules were amplified via PCR to generate sequencing-ready gDNA libraries. The libraries were then sequenced via next-generation sequencing (NEXTSEQ™ 550) and library size measured after demultiplexing. Results were reported as level of read representation, with increased read representation indicating greater confidence in accuracy of results.

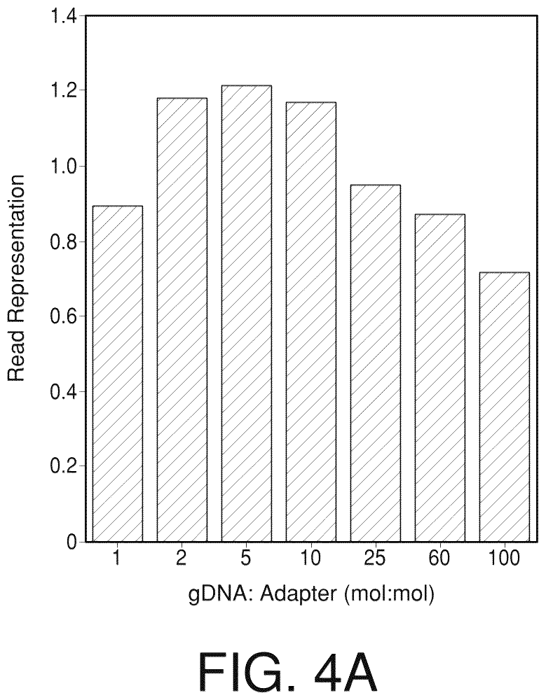

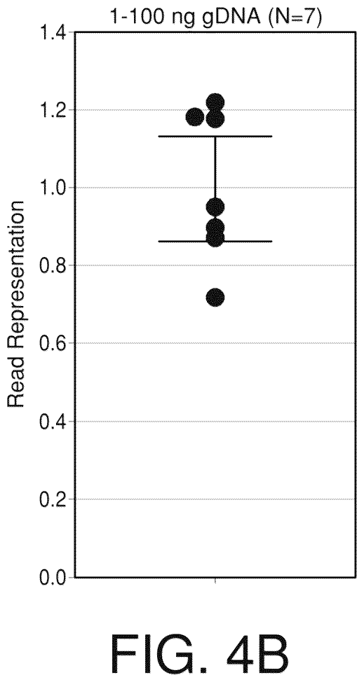

As shown in FIG. 4A and FIG. 4B, the level of read representation remained relatively uniform with low variation across seven different ratios of input gDNA to adapter ratios, even as mass input of the gDNA library is increased.

### Example 7: Normalization Test of Hybrid Circular Adapters with Cleavable Bases

This example demonstrates the ability of exemplary adapter conjugates (hybrid circular adapters) modified with cleavable bases to improve denaturation and minimize disruption of downstream PCR steps, and to be used to accurately sequence gDNA samples regardless of varying masses of the samples.

Hybrid circular adapters having cleavable uracil bases and having the sequences shown in Table 7, below, were generated according to methods described herein (e.g., in Example 3, above).

TABLE 7
Modified Adapter Sequences

| Adapter Name | Polynucleotide Sequence |
|---|---|
| 500-5-linked- | 3′-T*CTAGCCTTCTCGCAGCACA |
| 2sp18-v1-1U | TCCCTTTCTCACAU-5′-/Sp18/ |
| | /Sp18/-5′- |
| | ACACTCTTTCCCTACACGACGCTC |
| | TTCCGATC*T-3′ |
| | (SEQ ID NO: 39 in 3′-5′ |
| | direction)/Sp18//Sp18/ |
| | (SEQ ID NO: 36 in 5′-3′ |
| | direction) |
| 500-5-linked- | 3′-T*CTAGCCTTCTCGCAGCACA |
| 2sp18-v1-2U | TCCCTTTCTCACAUU-5′-/Sp18/ |
| | /Sp18/-5′- |
| | ACACTCTTTCCCTACACGACGCTCT |
| | TCCGATC*T-3′ |
| | (SEQ ID NO: 40 in the 3′-5′ |
| | direction)/Sp18//Sp18/(SEQ |
| | ID NO: 36 in the 5′-3′ |
| | direction) |
| 700-3-linked- | 5′-/5Phos/G*ATCGGAAGAGCACAC |
| 2sp18-v1-1U | GTCTGAACTCCAGTCACA-3′- |
| | /Sp18//Sp18/-3′- |
| | UCACTGACCTCAAGTCTGCACACGAGA |
| | AGGCTA* G/5Phos/-5′ |
| | /5Phos/(SEQ ID NO: 47 in the |
| | 5′-3′ direction)/Sp18//Sp18/ |
| | (SEQ ID NO: 48 in the |
| | 3′-5′ direction)/5Phos/ |

*indicates phosphorothioate bond.
“Sp18” indicates Spacer 18 (PEG).
“5Phos” indicates phosphorylation as a 5′ end modification.
Underlining indicates a cleavable base.

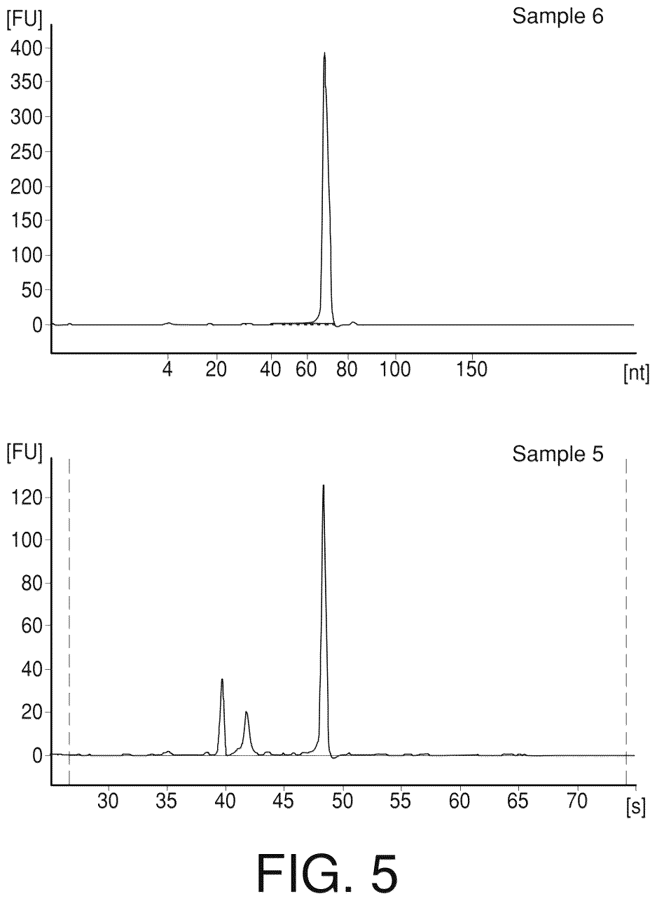

The adapters were then prepared and tested for sample normalization according to the methods described in Example 6, above, except that after ligation and before PCR, adapters were cleaved with USER enzyme, and cleavage was assessed and optimized based on bioanalyzer readout, as shown in FIG. 5.

### Example 8: Hybrid Circular Adapters with Duplex and Overhang Linkages

This example demonstrates the ability of exemplary adapter conjugates (hybrid circular adapters) modified to comprise duplex linkages and/or overhang linkages to improve denaturation, and to be used to accurately sequence gDNA samples regardless of varying masses of the samples.

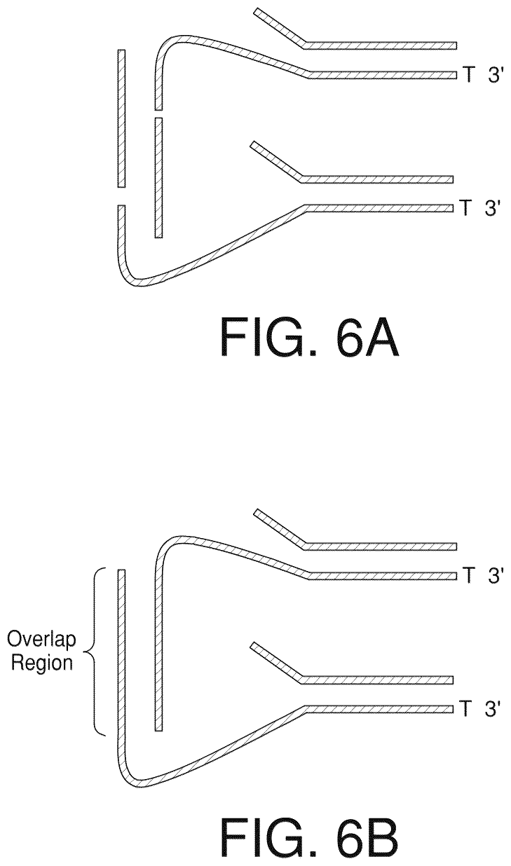

Hybrid circular adapters having duplex linkages or overhang linkages and having the sequences shown in Table 8, below, were generated according to methods described herein (e.g., Examples 4 and 5, above). The structures of the duplex and/or overhang linkages are illustrated in FIG. 6A and FIG. 6B.

TABLE 8
Modified Adapter Sequences

| | Polynucleotide |
|---|---|
| Adapter Name | Sequence (5′-3′) |
| 500-duplex- | /5-SpC3/TCGTCGCAATGG |
| linked- | TCTCACGAGGGCACGTAC |
| 2sp18-v1 | (SEQ ID NO: |
| | 42)/1-Sp18/CCAGGTATC |
| | GTGTAAGTAGCGA |
| | (SEQ ID NO: 43)/ |
| | I-Sp18/ |
| | ACACTCTTTCCCTACACGAC |
| | GCTCTTCCGATC*T (SEQ |
| | ID NO: 36) |
| 1-duplex- | /5-SpC3/GTACGTGCCCTC |
| linker-2sp18- | GTGAGACCATTGCGACGA |
| v1 | (SEQ ID NO: |
| | 44)/I-Sp18/ |
| | TCACAGCTGTGGATTGGATT |
| | GCCCTCCGAGTGAGCGTGGC |
| | (SEQ ID NO: 45)/ |
| | 3-SpC3/ |
| 2-duplex- | /5-SpC3/GTACGTGCCCTC |
| linker-2sp18- | GTGAGACCATTGCGACGA |
| v1 | (SEQ ID NO: 44)/ |
| | I-Sp18/ |
| | GCCACGCTCACTCGGAGGGC |
| | AATCCAATCCACAGCTGTGA |
| | (SEQ ID NO: 46)/ |
| | 3-SpC3/ |
| 1-500- | /5-SpC3/GTACGTGCCCTC |
| overhang- | GTGAGACCATTGCGACGA |
| linked- | (SEQ ID NO: 44)/ |
| 2sp18-v1 | I-Sp18/CCAGGTATC |
| | GTGTAAGTAGCGA (SEQ I |
| | D NO: 43)/I-Sp18/ |
| | ACACTCTTTCCCTACACGAC |
| | GCTCTTCCGATC*T (SEQ |
| | ID NO: 36) |
| 2-500- | /5-SpC3/TCGTCGCAATGG |
| overhang- | TCTCACGAGGGCACGTAC |
| linked- | (SEQ ID NO:42)/ |
| 2sp18-v1 | I-Sp18/CCAGGTATC |
| | GTGTAAGTAGCGA |
| | (SEQ ID NO: 43)/ |
| | I-Sp18/ |
| | ACACTCTTTCCCTACACGAC |
| | GCTCTTCCGATC*T (SEQ |
| | ID NO: 36) |

*indicates phosphorothioate bond.
“I-Sp18” indicates Spacer 18 (PEG) as an internal modification.
“5-SpC3” indicates Spacer 3 (C3) as a 5′ end modification.
“3-SpC3” indicates Spacer 3 (C3) as a 3′ end modification.

The adapters were blended and pooled at 10 μM final working concentration ratios and tested for sample normalization according to methods described in Example 6, above.

### Example 9: Determination of Adapter Flexibility for Intramolecular Connection

This example demonstrates the ability of flexible adapter conjugates (hybrid circular adapters) to be used to accurately sequence gDNA samples.

Flexible hybrid circular adapters minimize steric hindrance during the ligation process where adapters interact with library molecules, and thereby speed up the intramolecular ligation step for sequencing. However, it is important that the flexible adapters are ligating to library molecules on a one-on-one basis, as ligation to multiple library molecules would interfere with accuracy.

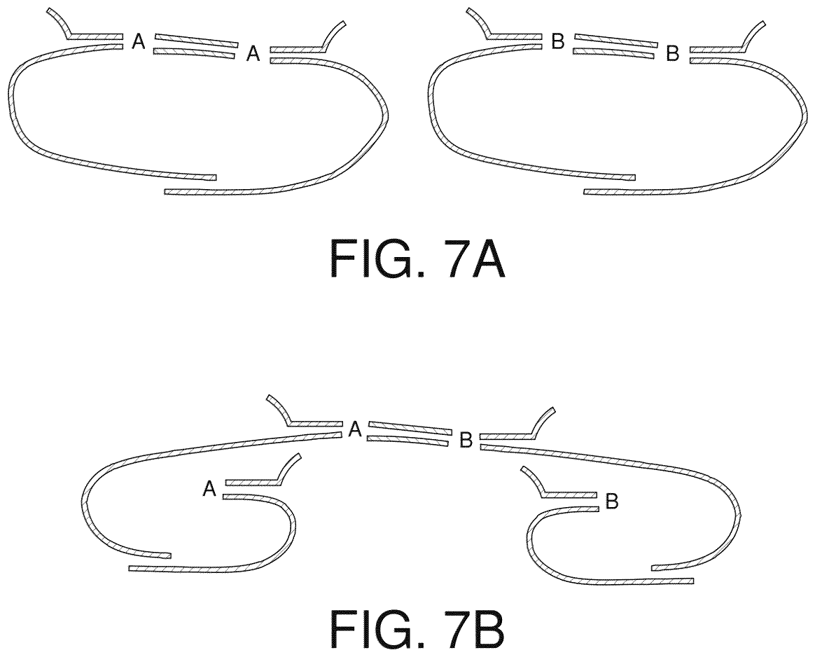

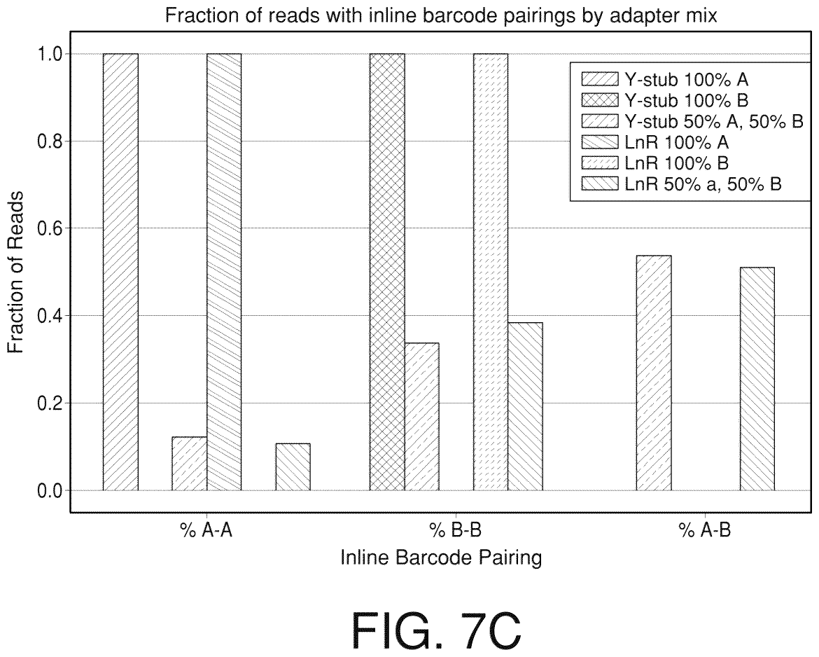

To validate the pairing of individual hybrid circular adapter molecules with library molecules on a one-to-one basis, hybrid circular adapters having internal (inline) barcodes (“barcoded adapters”) and having sequences as shown in Table 9A, below, were generated according to methods described herein (e.g., in Example 1, above). An assay was employed to assess cross-talk between the inline barcodes by quantifying the extent of converted library molecules linked to single adapters (see, e.g., illustration of inline barcode cross-talk in FIG. 7A and FIG. 7B). Barcoded adapters were then annealed and pooled for ligation with library molecules having sequences shown in Table 9B, below.

TABLE 9A
Barcoded Adapter Molecule Sequences

| | Polynucleotide Sequence |
|---|---|
| Name | (5′-3′) |
| 1-500- | /5-SpC3/GTACGTGCCCTCGTG |
| overhang- | AGACCATTGCGACGA |
| linked- | (SEQ ID NO: 44)/I-Sp18/ |
| 2sp18-GEN | CCAGGTATCGTGTAAGTAGCGA |
| | (SEQ ID NO: X)/I-Sp18/ |
| | ACACTCTTTCCCTACACGACGCT |
| | CTTCCGATCT(barcode)* T |
| | (SEQ ID NOs: 49 and 96-99) |
| 2-500- | /5-SpC3/TCGTCGCAATGGTCTC |
| overhang- | ACGAGGGCACGTAC |
| linked- | (SEQ ID NO: 42) |
| 2sp18-GEN | /I-Sp18/CCAGGTATCGTGTAAG |
| | TAGCGA (SEQ ID NO: 43)/ |
| | I-Sp18/ACACTCTTTCCCTACA |
| | CGACGCTCTTCCGATCT(barcode) |
| | * T (SEQ ID NOs: 49 and 96-99) |
| 700-STUB- | /5Phos/ |
| GEN-v1 | (barcode RC)AGATCGGAAGA |
| | GCACACGTCTGAACTCCAGTCA*C |
| | (SEQ ID NOs: 50 and 100-103) |

*indicates a phosphorothioate bond.
“5-SpC3” indicates Spacer 3 (C3) as a 5′ end modification.
“I-Sp18” indicates Spacer 18 (PEG) as an internal modification.
“/5Phos/” indicates phosphorylation as a 5′ end modification.

TABLE 9B
Library Molecule Sequences

| Name | Polynucleotide Sequence (5′-3′) |
|---|---|
| 1-500-overhang-linked- | /5-SpC3/GTACGTGCCCTCGTGAGACCATTGCGACGA (SEQ ID NO: |
| 2sp18-A | 44)/I-Sp18/CCAGGTATCGTGTAAGTAGCGA (SEQ ID NO: 43)/I-Sp18/ |
| | ACACTCTTTCCCTACACGACGCTCTTCCGATCTGTCGTA*T (SEQ |
| | ID NO: 51) |
| 2-500-overhang-linked- | /5-SpC3/TCGTCGCAATGGTCTCACGAGGGCACGTAC (SEQ ID NO: |
| 2sp18-A | 42)/I-Sp18/CCAGGTATCGTGTAAGTAGCGA (SEQ ID NO: 43)/I-Sp18/ |
| | ACACTCTTTCCCTACACGACGCTCTTCCGATCTGTCGTA*T (SEQ |
| | ID NO: 51) |
| 700-STUB-A-v1 | /5Phos/TACGACAGATCGGAAGAGCACACGTCTGAACTCCAGTCA*C |
| | (SEQ ID NO: 52) |
| 1-500-overhang-linked- | /5-SpC3/GTACGTGCCCTCGTGAGACCATTGCGACGA (SEQ ID NO: |
| 2sp18-B | 44)/I-Sp18/CCAGGTATCGTGTAAGTAGCGA (SEQ ID NO: 43)/I-Sp18/ |
| | ACACTCTTTCCCTACACGACGCTCTTCCGATCTAACGATG*T (SEQ |
| | ID NO: 53) |
| 2-500-overhang-linked- | /5-SpC3/TCGTCGCAATGGTCTCACGAGGGCACGTAC (SEQ ID NO: |
| 2sp18-B | 42)/I-Sp18/CCAGGTATCGTGTAAGTAGCGA (SEQ ID NO: 43)/I-Sp18/ |
| | ACACTCTTTCCCTACACGACGCTCTTCCGATCTAACGATG*T (SEQ |
| | ID NO: 53) |
| 700-STUB-B-v1 | /5Phos/CATCGTTAGATCGGAAGAGCACACGTCTGAACTCCAGTCA* |
| | C (SEQ ID NO: 54) |

*indicates a phosphorothioate bond.
“5-SpC3” indicates Spacer 3 (C3) as a 5′ end modification.
“I-Sp18” indicates Spacer 18 (PEG) as an internal modification.
“/5Phos/” indicates phosphorylation as a 5′ end modification.

Next, the ligated library molecules underwent next-generation sequencing and paired-end read data was parsed to identify inline barcodes and analyze amount of output cross-matching. As shown in FIG. 7C, cross-matching of inline barcodes signifies instances where library molecules were ligated to two distinct circular adapters.

### Example 10: Hybrid Circular Adapters with Increased Flexibility Linkages

This example demonstrates the effect of various factors (overhang length, base content, melting temperature, linker type, linker length, and linker amount) on adapter performance in terms of increasing ligation rate, decreasing cross-talk, and normalizing samples.

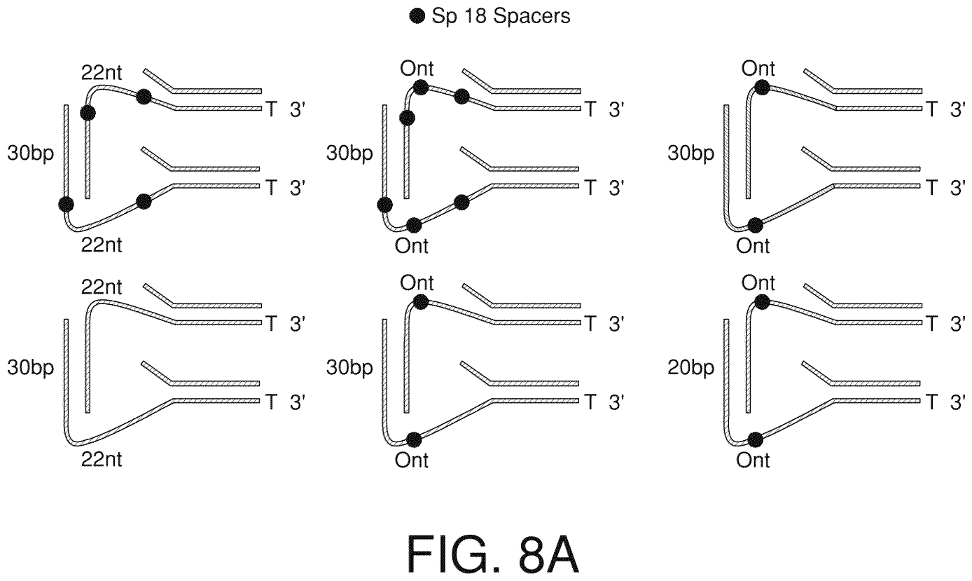

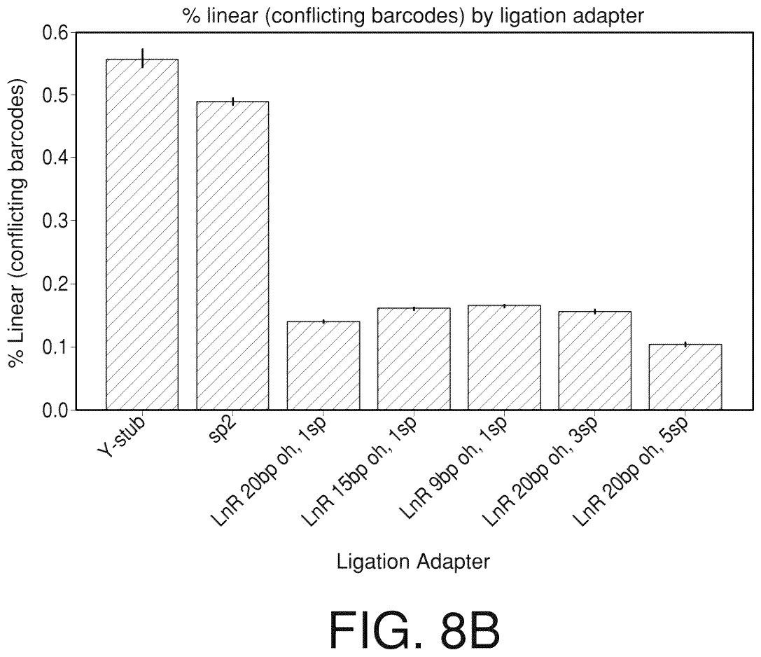

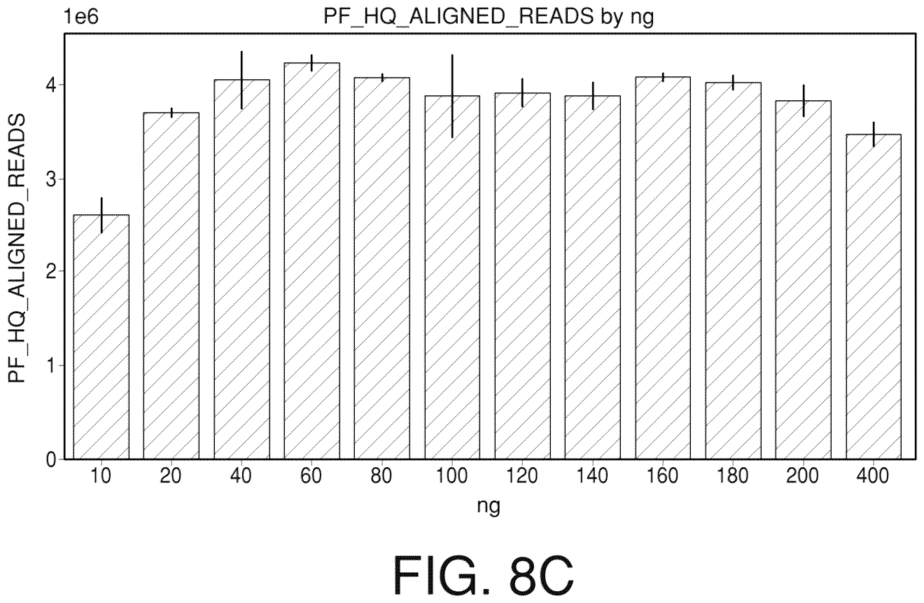

Based on the results described in Example 9, above, two adapters (SEQ ID NO: 51 and SEQ ID NO: 53) were selected for testing. These adapters were then modified to generate the adapters shown in Table 10, below. Iterations of adapter overhang length, base content, melting temperature, linker types, linker lengths, and linker amounts were evaluated. See, e.g., FIG. 8A. Criteria of the evaluation included determining adapters that resulted in the most favorable (e.g., strongest) intramolecular connection as measured using the assay described in Example 9, above. As shown in FIG. 8B, the different iterations of the adapters demonstrated different amounts of cross-talk.

Adapters which promoted the least amount of cross-talk were selected and tested for normalization according the methods described herein. As shown in FIG. 8C, normalization and similar conversion of library molecules were observed within a 10-fold range of 20-200 ng total input mass into library preparation and ligation with the selected hybrid circular adaptors.

TABLE 10
Adapter Sequences

| Adapter Name | Polynucleotide Sequence (5′-3′) |
|---|---|
| 1-500-overhang- | /5-SpC3/CAAAGTGCGATTCTACGACC (SEQ ID NO: 55)/I-Sp18/ |
| linked-A-v2 | ACACTCTTTCCCTACACGACGCTCTTCCGATCTGTCGTA*T (SEQ ID |
| | NO: 51) |
| 2-500-overhang- | /5-SpC3/GGTCGTAGAATCGCACTTTG (SEQ ID NO: 56)/I-Sp18/ |
| linked-A-v2 | ACACTCTTTCCCTACACGACGCTCTTCCGATCTGTCGTA*T (SEQ ID |
| | NO: 51) |
| 1-500-overhang- | /5-SpC3/CAAAGTGCGATTCTACGACC (SEQ ID NO: 55)/I-Sp18/ |
| linked-B-v2 | ACACTCTTTCCCTACACGACGCTCTTCCGATCTAACGATG*T (SEQ ID |
| | NO: 53) |
| 2-500-overhang- | /5-SpC3/GGTCGTAGAATCGCACTTTG (SEQ ID NO: 56)/I-Sp18/ |
| linked-B-v2 | ACACTCTTTCCCTACACGACGCTCTTCCGATCTAACGATG* T (SEQ ID |
| | NO: 53) |
| 1-500-overhang- | /5-SpC3/TGCGATTCTACGACC (SEQ ID NO: 57)/I-Sp18/ |
| linked-A-v3 | ACACTCTTTCCCTACACGACGCTCTTCCGATCTGTCGTA*T (SEQ ID |
| | NO: 51) |
| 2-500-overhang- | /5-SpC3/GGTCGTAGAATCGCA (SEQ ID NO: 58)/I-Sp18/ |
| linked-A-v3 | ACACTCTTTCCCTACACGACGCTCTTCCGATCTGTCGTA*T (SEQ ID |
| | NO: 51) |
| 1-500-overhang- | /5-SpC3/TGCGATTCTACGACC (SEQ ID NO: 57)/I-Sp18/ |
| linked-B-v3 | ACACTCTTTCCCTACACGACGCTCTTCCGATCTAACGATG*T (SEQ ID |
| | NO: 53) |
| 2-500-overhang- | /5-SpC3/GGTCGTAGAATCGCA (SEQ ID NO: 58)/I-Sp18/ |
| linked-B-v3 | ACACTCTTTCCCTACACGACGCTCTTCCGATCTAACGATG* T (SEQ ID |
| | NO: 53) |
| 1-500-overhang- | /5-SpC3/CCCGGCGACC (SEQ ID NO: 59)/I-Sp18/ |
| linked-A-v4 | ACACTCTTTCCCTACACGACGCTCTTCCGATCTGTCGTA*T (SEQ ID |
| | NO: 51) |
| 2-500-overhang- | /5-SpC3/GGTCGCCGGG (SEQ ID NO: 60)/I-Sp18/ |
| linked-A-v4 | ACACTCTTTCCCTACACGACGCTCTTCCGATCTGTCGTA*T (SEQ ID |
| | NO: 51) |
| 1-500-overhang- | /5-SpC3/CCCGGCGACC (SEQ ID NO: 59)/I-Sp18/ |
| linked-B-v4 | ACACTCTTTCCCTACACGACGCTCTTCCGATCTAACGATG*T (SEQ ID |
| | NO: 53) |
| 2-500-overhang- | /5-SpC3/GGTCGCCGGG (SEQ ID NO: 60)/I-Sp18/ |
| linked-B-v4 | ACACTCTTTCCCTACACGACGCTCTTCCGATCTAACGATG*T (SEQ ID |
| | NO: 53) |
| 1-500-overhang- | /5-SpC3/CAAAGTGCGATTCTACGACC (SEQ ID NO: 55)/I-Sp18//I-Sp18// |
| linked-A-v5 | I-Sp18/ACACTCTTTCCCTACACGACGCTCTTCCGATCTGTCGTA*T |
| | (SEQ ID NO: 51) |
| 2-500-overhang- | /5-SpC3/GGTCGTAGAATCGCACTTTG (SEQ ID NO: 56)/1-Sp18//I-Sp18// |
| linked-A-v5 | I-Sp18/ACACTCTTTCCCTACACGACGCTCTTCCGATCTGTCGTA*T |
| | (SEQ ID NO: 51) |
| 1-500-overhang- | /5-SpC3/CAAAGTGCGATTCTACGACC (SEQ ID NO: 55)/I-Sp18//I-Sp18// |
| linked-B-v5 | I-Sp18/ACACTCTTTCCCTACACGACGCTCTTCCGATCTAACGATG*T |
| | (SEQ ID NO: 53) |
| 2-500-overhang- | /5-SpC3/GGTCGTAGAATCGCACTTTG (SEQ ID NO: 56)/I-Sp18//I-Sp18// |
| linked-B-v5 | I-Sp18/ACACTCTTTCCCTACACGACGCTCTTCCGATCTAACGATG*T |
| | (SEQ ID NO: 53) |
| 1-500-overhang- | /5-SpC3/CAAAGTGCGATTCTACGACC (SEQ ID NO: 55)/I-Sp18//I-Sp18// |
| linked-A-v6 | I-Sp18//I-Sp18//I-Sp18/ |
| | ACACTCTTTCCCTACACGACGCTCTTCCGATCTGTCGTA*T (SEQ ID |
| | NO: 51) |
| 2-500-overhang- | /5-SpC3/GGTCGTAGAATCGCACTTTG (SEQ ID NO: 56)/I-Sp18//I-Sp18// |
| linked-A-v6 | I-Sp18//I-Sp18//I-Sp18/ |
| | ACACTCTTTCCCTACACGACGCTCTTCCGATCTGTCGTA*T (SEQ ID |
| | NO: 51) |
| 1-500-overhang- | /5-SpC3/CAAAGTGCGATTCTACGACC (SEQ ID NO: 55)/I-Sp18//I-Sp18// |
| linked-B-v6 | I-Sp18//I-Sp18//I-Sp18/ |
| | ACACTCTTTCCCTACACGACGCTCTTCCGATCTAACGATG* T (SEQ ID |
| | NO: 53) |
| 2-500-overhang- | /5-SpC3/GGTCGTAGAATCGCACTTTG (SEQ ID NO: 56)/I-Sp18//I-Sp18// |
| linked-B-v6 | I-Sp18//I-Sp18//I-Sp18/ |
| | ACACTCTTTCCCTACACGACGCTCTTCCGATCTAACGATG*T (SEQ ID |
| | NO: 53) |
| 1-500-overhang- | /5-SpC3/CAAAGTGCGATTCTACGACC (SEQ ID NO: 55)/I-Sp18/ |
| linked-A-v7 | TCTCACAGTGACACTCTTTCCCTACACGACGCTCTTCCGATCTGTCGT |
| | A*T (SEQ ID NO: 61) |
| 2-500-overhang- | /5-SpC3/GGTCGTAGAATCGCACTTTG (SEQ ID NO: 56)/I-Sp18/ |
| linked-A-v7 | TCTCACAGTGACACTCTTTCCCTACACGACGCTCTTCCGATCTGTCGT |
| | A*T (SEQ ID NO: 61) |
| 1-500-overhang- | /5-SpC3/CAAAGTGCGATTCTACGACC (SEQ ID NO: 55)/I-Sp18/ |
| linked-B-v7 | TCTCACAGTGACACTCTTTCCCTACACGACGCTCTTCCGATCTAACGA |
| | TG*T (SEQ ID NO: 62) |
| 2-500-overhang- | /5-SpC3/GGTCGTAGAATCGCACTTTG (SEQ ID NO: 56)/I-Sp18/ |
| linked-B-v7 | TCTCACAGTGACACTCTTTCCCTACACGACGCTCTTCCGATCTAACGA |
| | TG*T (SEQ ID NO: 62) |
| 1-500-overhang- | /5-SpC3/CAAAGTGCGATTCTACGACC (SEQ ID NO: 55)/I-Sp18/ |
| linked-A-v8 | TCTCACAGTGAACTACGCCTACACTCTTTCCCTACACGACGCTCTTCC |
| | GATCTGTCGTA* T (SEQ ID NO: 63) |
| 2-500-overhang- | /5-SpC3/GGTCGTAGAATCGCACTTTG (SEQ ID NO: 56)/I-Sp18/ |
| linked-A-v8 | TCTCACAGTGAACTACGCCTACACTCTTTCCCTACACGACGCTCTTCC |
| | GATCTGTCGTA*T (SEQ ID NO: 63) |
| 1-500-overhang- | /5-SpC3/CAAAGTGCGATTCTACGACC (SEQ ID NO: 55)/I-Sp18/ |
| linked-B-v8 | TCTCACAGTGAACTACGCCTACACTCTTTCCCTACACGACGCTCTTCC |
| | GATCTAACGATG*T (SEQ ID NO: 64) |
| 2-500-overhang- | /5-SpC3/GGTCGTAGAATCGCACTTTG (SEQ ID NO: 56)/I-Sp18/ |
| linked-B-v8 | TCTCACAGTGAACTACGCCTACACTCTTTCCCTACACGACGCTCTTCC |
| | GATCTAACGATG*T (SEQ ID NO: 64) |
| 1-500-overhang- | /5-SpC3/CAAAGTGCGATTCTACGACCAACTACGCCT (SEQ ID NO: 65)/ |
| linked-A-v9 | I-Sp18/ |
| | TCTCACAGTGACACTCTTTCCCTACACGACGCTCTTCCGATCTGTCGT |
| | A*T (SEQ ID NO: 61) |
| 2-500-overhang- | /5-SpC3/GGTCGTAGAATCGCACTTTGAACTACGCCT (SEQ ID NO: 66)/ |
| linked-A-v9 | I-Sp18/ |
| | TCTCACAGTGACACTCTTTCCCTACACGACGCTCTTCCGATCTGTCGT |
| | A*T (SEQ ID NO: 61) |
| 1-500-overhang- | /5-SpC3/CAAAGTGCGATTCTACGACCAACTACGCCT (SEQ ID NO: 65)/ |
| linked-B-v9 | I-Sp18/ |
| | TCTCACAGTGACACTCTTTCCCTACACGACGCTCTTCCGATCTAACGA |
| | TG* T (SEQ ID NO: 62) |
| 2-500-overhang- | /5-SpC3/GGTCGTAGAATCGCACTTTGAACTACGCCT (SEQ ID NO: 66)/ |
| linked-B-v9 | I-Sp18/ |
| | TCTCACAGTGACACTCTTTCCCTACACGACGCTCTTCCGATCTAACGA |
| | TG*T (SEQ ID NO: 62) |
| 1-500-overhang- | /5-SpC3/CAAAGTGCGATTCTACGACC (SEQ ID NO: 55)/I-Sp18/ |
| linked-A-v10 | TCTCACAGTGAACTACGCCTAGGTTTATGGAATACAGCTAACACTCTT |
| | TCCCTACACGACGCTCTTCCGATCTGTCGTA*T (SEQ ID NO: 67) |
| 2-500-overhang- | /5-SpC3/GGTCGTAGAATCGCACTTTG (SEQ ID NO: 56)/I-Sp18/ |
| linked-A-v10 | TCTCACAGTGAACTACGCCTAGGTTTATGGAATACAGCTAACACTCTT |
| | TCCCTACACGACGCTCTTCCGATCTGTCGTA* T (SEQ ID NO: 67) |
| 1-500-overhang- | /5-SpC3/CAAAGTGCGATTCTACGACC (SEQ ID NO: 55)/I-Sp18/ |
| linked-B-v10 | TCTCACAGTGAACTACGCCTAGGTTTATGGAATACAGCTACACTCTTT |
| | CCCTACACGACGCTCTTCCGATCTAACGATG*T (SEQ ID NO: 68) |
| 2-500-overhang- | /5-SpC3/GGTCGTAGAATCGCACTTTG (SEQ ID NO: 56)/I-Sp18/ |
| linked-B-v10 | TCTCACAGTGAACTACGCCTAGGTTTATGGAATACAGCTACACTCTTT |
| | CCCTACACGACGCTCTTCCGATCTAACGATG*T (SEQ ID NO: 68) |

*indicates a phosphorothioate bond.
“5-SpC3” indicates Spacer 3 (C3) as a 5′ end modification.
“I-Sp18” indicates Spacer 18 (PEG) as an internal modification.

### Example 11: Hybrid Circular Adapters with Inline Barcodes

This example demonstrates the ability of exemplary adapter conjugates (hybrid circular adapters) to incorporate inline barcodes for use in sample normalization during next-generation sequencing without affecting accuracy.

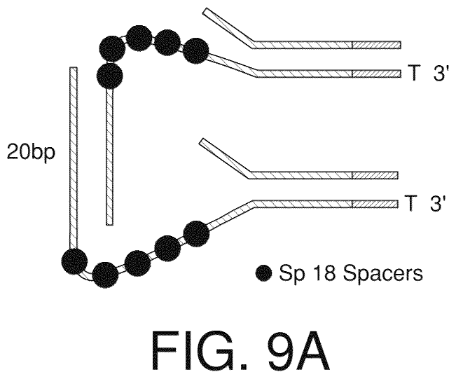

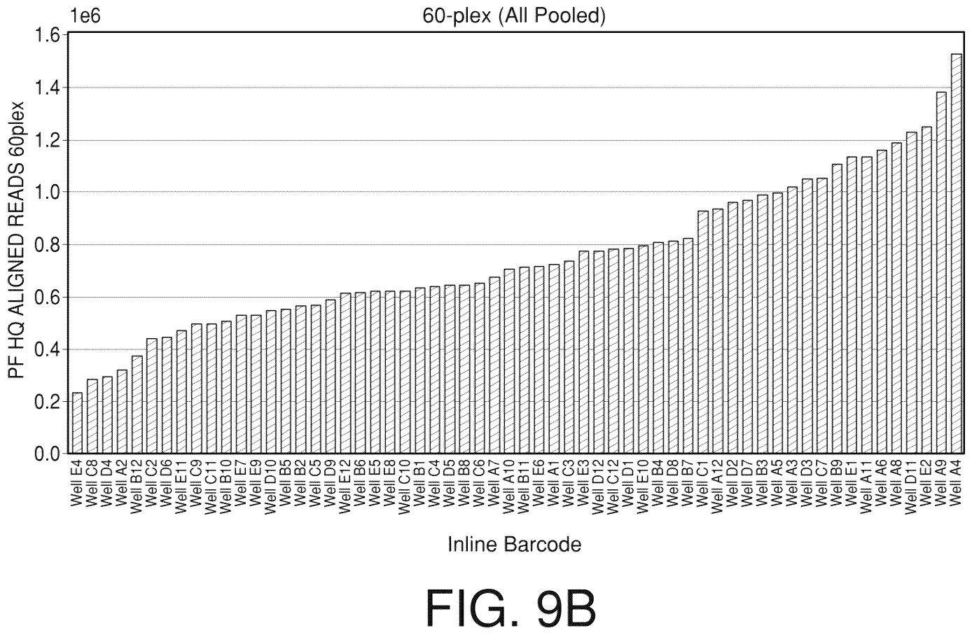

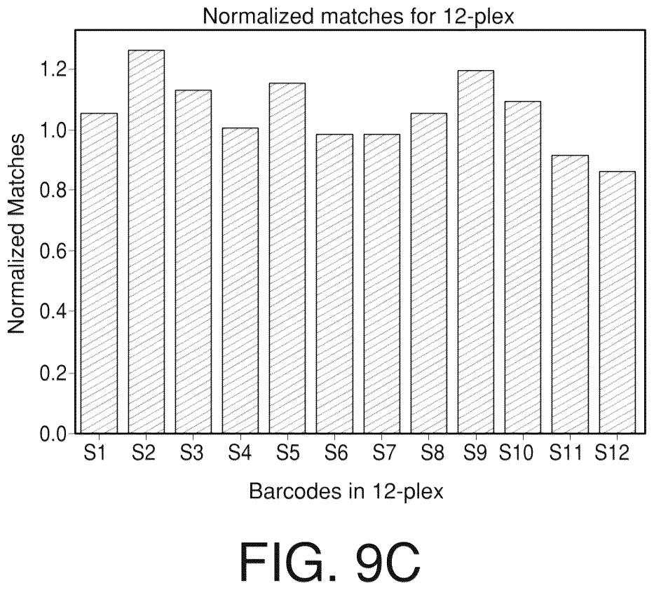

Sixty inline barcode sequences were designed to have intermediate GC content, to have a Hamming distance of at least 2 away from each other barcode, and to have a length of 6-7 bases. As shown in FIG. 9B, accuracy of sequencing results (measured via read representation) varied across the sixty barcodes. Of these sixty inline barcode sequences, a set of twelve sequences were selected and incorporated into each of three different adapters, as shown in Table 11, below. An illustration of the structure of the adapters with inline barcodes is shown in FIG. 9A. The adapters were then blended in a single pool at a final concentration of 10 μM, ligated with gDNA samples, and tested for sample normalization according to the methods described herein (e.g., in Example 6, above). In the testing of the selected barcodes, each sample had a fixed mass input to assess barcode uniformity.

TABLE 11
Barcode and Adapter Sequences

| | | 1-500-overhang- | 2-500-overhang- | |
|---|---|---|---|---|
| Barcode | Barcode | linked Adapter | linked Adapter | 700-overhang-linked |
| ID | Sequence | Sequence | Sequence | Adapter Sequence |
| S1 | ACATCA | /5-SpC3/ | /5-SpC3/ | /5Phos/ |
| | (SEQ ID | CAAAGTGCGATTCT | GGTCGTAGAATCGC | TGATGTAGATCGGA |
| | NO: 69) | ACGACC (SEQ ID NO: | ACTTTG (SEQ ID NO: | AGAGCACACGTCTG |
| | | 55)/ | 56)/ | AACTCCAGTCA*C |
| | | I-Sp18// | I-Sp18// | (SEQ ID NO: 101) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTACATCA*T | GATCTACATCA* T | |
| | | (SEQ ID NO: 89) | (SEQ ID NO: 96) | |
| S2 | ACGACT | /5-SpC3/ | /5-SpC3/ | /5Phos/ |
| | (SEQ ID | CAAAGTGCGATTCT | GGTCGTAGAATCGC | AGTCGTAGATCGGA |
| | NO: 70) | ACGACC (SEQ ID NO: | ACTTTG (SEQ ID NO: | AGAGCACACGTCTG |
| | | 55)/ | 56)/ | AACTCCAGTCA*C |
| | | I-Sp18// | I-Sp18// | (SEQ ID NO: 102) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTACGACT*T | GATCTACGACT*T | |
| | | (SEQ ID NO: 90) | (SEQ ID NO: 90) | |
| S3 | AGGATAA | /5-SpC3/ | /5-SpC3/ | /5Phos/ |
| | (SEQ ID | CAAAGTGCGATTCT | GGTCGTAGAATCGC | TTATCCTAGATCGG |
| | NO: 71) | ACGACC (SEQ ID NO: | ACTTTG (SEQ ID NO: | AAGAGCACACGTCT |
| | | 55)/ | 56)/ | GAACTCCAGTCA*C |
| | | I-Sp18// | I-Sp18// | (SEQ ID NO: 103) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTAGGATAA*T | GATCTAGGATAA* T | |
| | | (SEQ ID NO: 91) | (SEQ ID NO: 91) | |
| S4 | ATGGTGA | /5-SpC3/ | /5-SpC3/ | /5Phos/ |
| | (SEQ ID | CAAAGTGCGATTCT | GGTCGTAGAATCGC | TCACCATAGATCGG |
| | NO: 72) | ACGACC | ACTTTG | AAGAGCACACGTCT |
| | | (SEQ ID NO: | (SEQ ID NO: | GAACTCCAGTCA*C |
| | | 55)/ | 56)/ | (SEQ ID NO: 104) |
| | | I-Sp18// | I-Sp18// | |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTATGGTGA*T | GATCTATGGTGA*T | |
| | | (SEQ ID NO: 92) | (SEQ ID NO: 92) | |
| S5 | CAACTG | /5-SpC3/ | /5-SpC3/ | /5Phos/ |
| | (SEQ ID | CAAAGTGCGATTCT | GGTCGTAGAATCGC | CAGTTGAGATCGGA |
| | NO: 73) | ACGACC | ACTTTG | AGAGCACACGTCTG |
| | | (SEQ ID NO: 55)/ | (SEQ ID NO: | AACTCCAGTCA*C |
| | | I-Sp18// | 56)/ | (SEQ ID NO: 105) |
| | | I-Sp18// | I-Sp18// | |
| | | I-Sp18// | I-Sp18// | |
| | | I-Sp18// | I-Sp18// | |
| | | I-Sp18/ | I-Sp18// | |
| | | ACACTCTTTCCCTA | I-Sp18/ | |
| | | CACGACGCTCTTCC | ACACTCTTTCCCTA | |
| | | GATCTCAACTG*T | CACGACGCTCTTCC | |
| | | (SEQ ID NO: 93) | GATCTCAACTG*T | |
| | | | (SEQ ID NO: 93) | |
| S6 | CCGATG | /5-SpC3/ | /5-SpC3/ | /5Phos/ |
| | (SEQ ID | CAAAGTGCGATTCT | GGTCGTAGAATCGC | CATCGGAGATCGGA |
| | NO: 74) | ACGACC (SEQ ID NO: | ACTTTG (SEQ ID NO: | AGAGCACACGTCTG |
| | | 55)/ | 56)/ | AACTCCAGTCA*C |
| | | I-Sp18// | I-Sp18// | (SEQ ID NO: 106) |
| | | I-Sp18// | I-Sp18// | |
| | | I-Sp18// | I-Sp18// | |
| | | I-Sp18// | I-Sp18// | |
| | | I-Sp18// | I-Sp18/ | |
| | | ACACTCTTTCCCTA | ACACTCTTTCCCTA | |
| | | CACGACGCTCTTCC | CACGACGCTCTTCC | |
| | | GATCTCCGATG*T | GATCTCCGATG*T | |
| | | (SEQ ID NO: 94) | (SEQ ID NO: 94) | |
| S7 | CTGGTT | /5-SpC3/ | /5-SpC3/ | /5Phos/ |
| | (SEQ ID | CAAAGTGCGATTCT | GGTCGTAGAATCGC | AACCAGAGATCGGA |
| | NO: 75) | ACGACC (SEQ ID NO: | ACTTTG (SEQ ID | AGAGCACACGTCTG |
| | | 55)/ | NO: 56)/ | AACTCCAGTCA*C |
| | | I-Sp18// | I-Sp18// | (SEQ ID NO: 107) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTCTGGTT*T | GATCTCTGGTT*T | |
| | | (SEQ ID NO: 95) | (SEQ ID NO: 95) | |
| S8 | GAAGCA | /5-SpC3/ | /5-SpC3/ | /5Phos/ |
| | (SEQ ID | CAAAGTGCGATTCT | GGTCGTAGAATCGC | TGCTTCAGATCGGA |
| | NO: 76) | ACGACC (SEQ ID NO: | ACTTTG (SEQ ID | AGAGCACACGTCTG |
| | | 55)/ | NO: 56)/ | AACTCCAGTCA*C |
| | | I-Sp18// | I-Sp18// | (SEQ ID NO: 108) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTGAAGCA* T | GATCTGAAGCA*T | |
| | | (SEQ ID NO: 96) | (SEQ ID NO: 96) | |
| S9 | GTAGCCA | /5-SpC3/ | /5-SpC3/ | /5Phos/ |
| | (SEQ ID | CAAAGTGCGATTCT | GGTCGTAGAATCGC | TGGCTACAGATCGG |
| | NO: 77) | ACGACC (SEQ ID NO: | ACTTTG (SEQ ID NO: | AAGAGCACACGTCT |
| | | 55)/ | 56)/ | GAACTCCAGTCA*C |
| | | I-Sp18// | I-Sp18// | (SEQ ID NO: 109) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTGTAGCCA*T | GATCTGTAGCCA*T | |
| | | (SEQ ID NO: 97) | (SEQ ID NO: 97) | |
| S10 | TCCTGAC | /5-SpC3/ | /5-SpC3/ | /5Phos/ |
| | (SEQ ID | CAAAGTGCGATTCT | GGTCGTAGAATCGC | GTCAGGAAGATCGG |
| | NO: 78) | ACGACC (SEQ ID NO: | ACTTTG (SEQ ID NO: | AAGAGCACACGTCT |
| | | 55)/ | 56)/ | GAACTCCAGTCA*C |
| | | I-Sp18// | I-Sp18// | (SEQ ID NO: 110) |
| | | I-Sp18// | -Sp18// | |
| | | I-Sp18// | -Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTTCCTGAC*T | GATCTTCCTGAC*T | |
| | | (SEQ ID NO: 98) | (SEQ ID NO: 98) | |
| S11 | TCTTGG | /5-SpC3/ | /5-SpC3/ | /5Phos/ |
| | (SEQ ID | CAAAGTGCGATTCT | GGTCGTAGAATCGC | CCAAGAAGATCGGA |
| | NO: 79) | ACGACC (SEQ ID NO: | ACTTTG (SEQ ID NO: | AGAGCACACGTCTG |
| | | 55)/ | 56)/ | AACTCCAGTCA*C |
| | | I-Sp18// | I-Sp18// | (SEQ ID NO: 111) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTTCTTGG*T | GATCTTCTTGG*T | |
| | | (SEQ ID NO: 99) | (SEQ ID NO: 99) | |
| S12 | TGGCTT | /5-SpC3/ | /5-SpC3/ | /5Phos/ |
| | (SEQ ID | CAAAGTGCGATTCT | GGTCGTAGAATCGC | AAGCCAAGATCGGA |
| | NO: 80) | ACGACC (SEQ ID NO: | ACTTTG (SEQ ID NO: | AGAGCACACGTCTG |
| | | 55)/ | 56)/ | AACTCCAGTCA*C |
| | | I-Sp18// | I-Sp18// | (SEQ ID NO: 112) |
| | | I-Sp18// | I-Sp18// | |
| | | I-Sp18// | I-Sp18// | |
| | | I-Sp18// | I-Sp18// | |
| | | I-Sp18/ | I-Sp18/ | |
| | | ACACTCTTTCCCTA | ACACTCTTTCCCTA | |
| | | CACGACGCTCTTCC | CACGACGCTCTTCC | |
| | | GATCTTGGCTT*T | GATCTTGGCTT*T | |
| | | (SEQ ID NO: 100) | (SEQ ID NO: 100) | |

*indicates a phosphorothioate bond.
“5-Sp3C” indicates Spacer 3 (C3) as a 5′ end modification.
“I-Sp18” indicates Spacer 18 (PEG) as an internal modification.

As shown in FIG. 9C, conversion rates of molecules were similar across the twelve selected barcodes.

### Example 12: Inline Barcodes for PCR Artifact Removals

This example demonstrates the use of exemplary inline barcodes as described herein to assess the presence of artifacts during the PCR step of next generation sequencing.

An initial screening of 32 inline barcode sequences was performed using Y-adaptor structures where individual barcodes were ligated to DNA library samples in individual isolated wells. Post pooling, solid phase immobilization (SPRI), and PCR amplification into sequencing, single barcodes were expected for every sample or read sequenced. Any molecules determined to contain mismatched (cross-talked) inline barcodes may be attributed to artifacts generated in the processing steps downstream of ligation. Such artifacts may be removed in silico for greater downstream analysis accuracy.

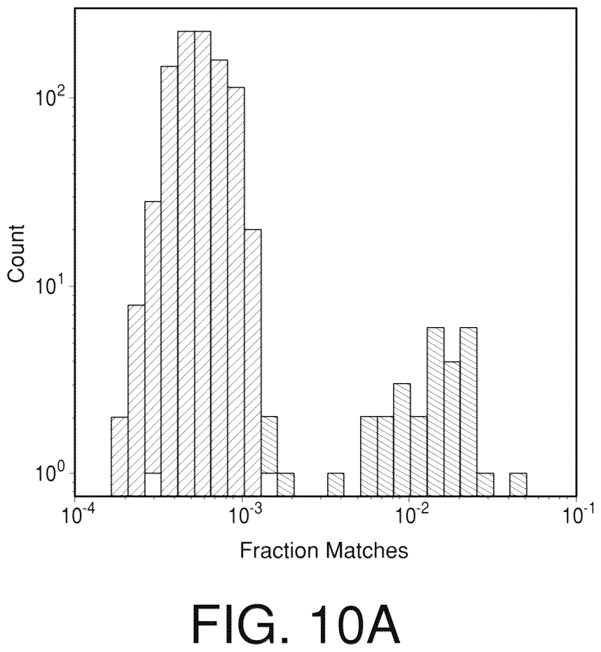

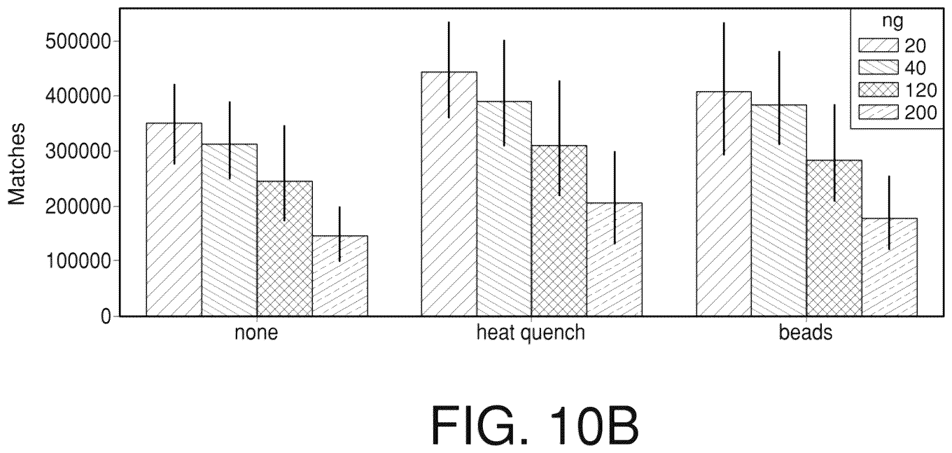

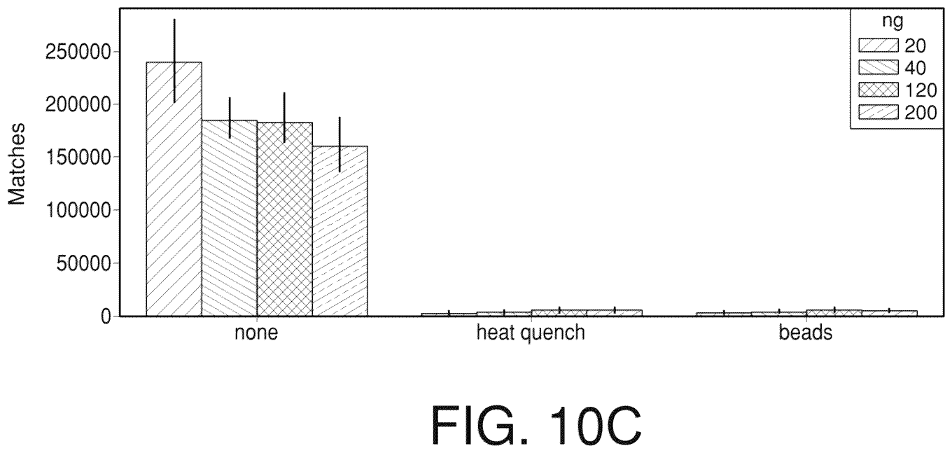

All possible barcodes (1024) were split into matched barcodes (32) and mismatched (cross-talked) barcodes (992), as shown in the histogram of FIG. 10A. As shown in FIG. 10B, initial testing of the pooling ligation reaction yielded high cross-talked barcodes. After individual bead purification or a heat denaturation step after ligation, the cross-talked barcodes were reduced or eliminated, as shown in FIG. 10C. Thus, the inline barcodes were used to both identify and eliminate artifacts during the PCR process.

### Example 13: High-throughput Library Preparation Workflow with Adapters

This example demonstrates the ability of exemplary barcoded adapters as described herein to pool samples directly after normalization and handle multiple samples sequenced in parallel.

Barcoded adapters described in Example 12, above, were used to execute a library workflow as described herein. In the workflow, samples of human gDNA and maize gDNA were enzymatically fragmented, normalized, and tagged with the adapters. The mass inputs of the gDNA samples were varied across a range of 40 ng to 200 ng. Fragmented gDNA were end repaired and dA-tailed for ligation with the barcoded adaptors. Ligation reactions were heat-denatured and pooled together for a SPRI cleanup at a 0.8× ratio. The eluted pool was subjected to twelve cycles of PCR reaction and then next-generation sequencing.

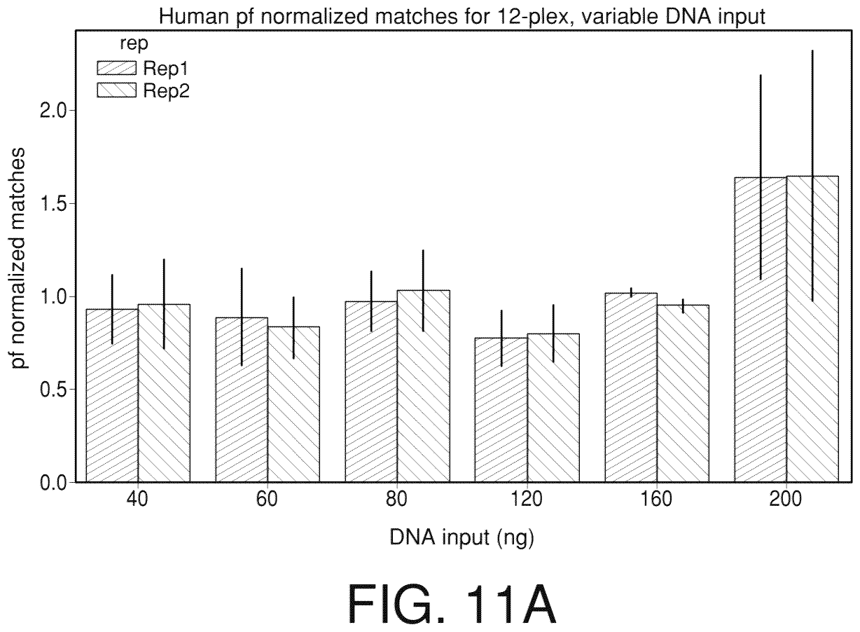

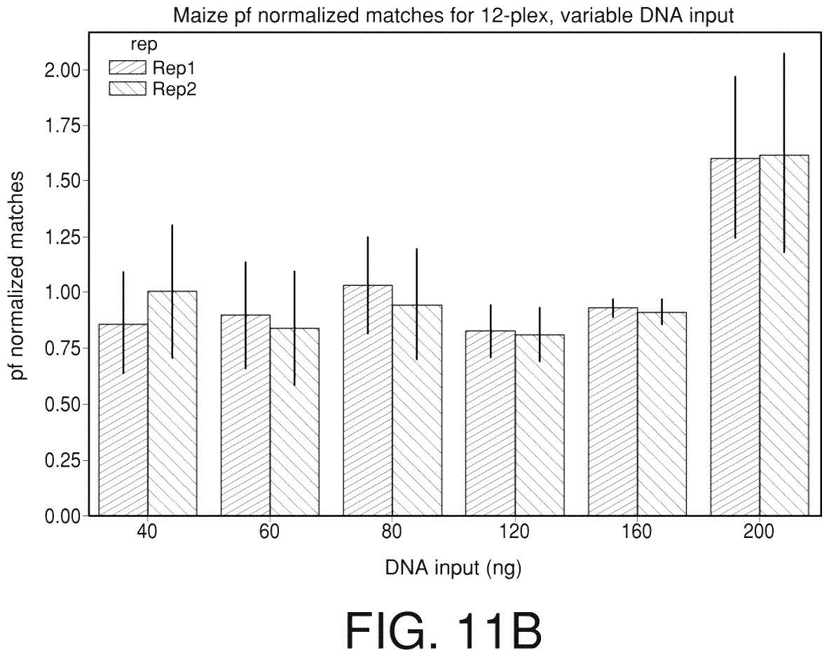

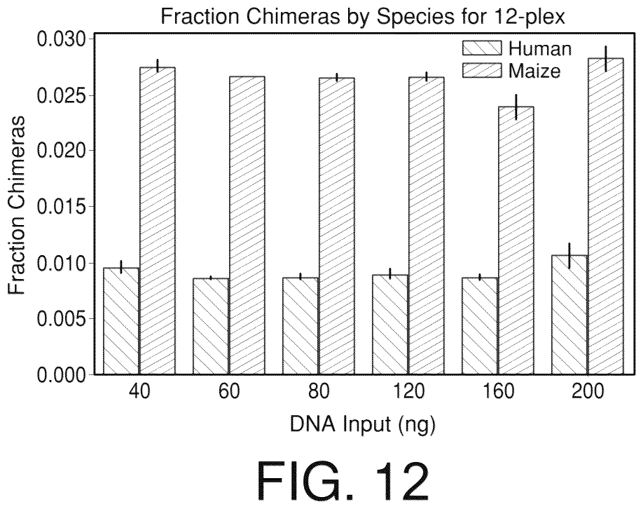

As shown in FIG. 11A (human samples) and FIG. 11B (maize samples), for both human and maize gDNA samples, passing filter normalized matches were relatively uniform across the range of mass inputs. As shown in FIG. 12, fraction chimera remained relatively uniform for both species, with maize samples demonstrating consistently greater fraction chimera than human samples.

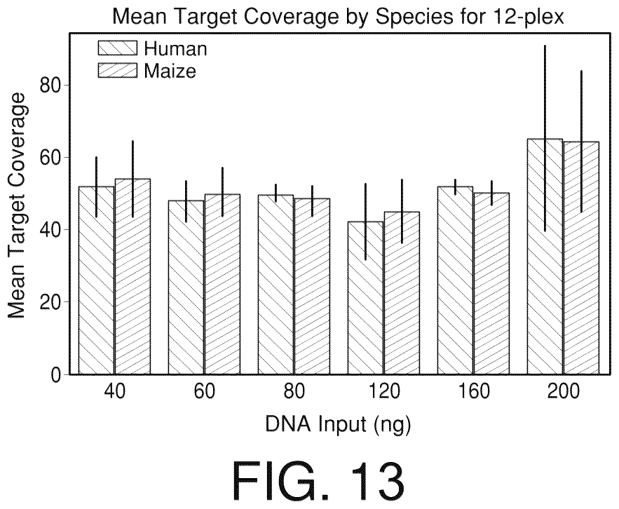

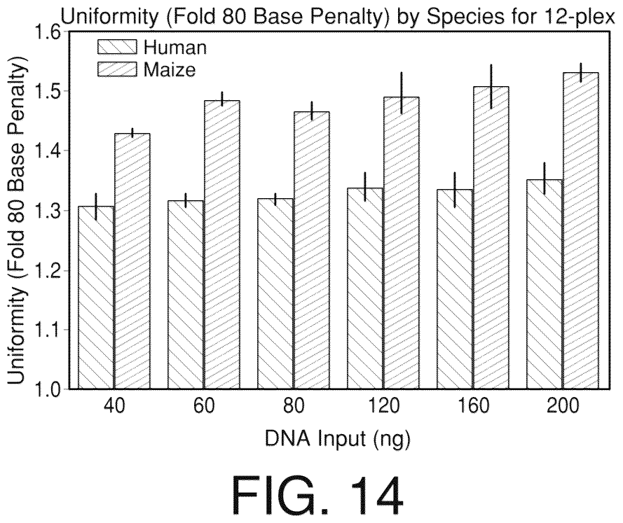

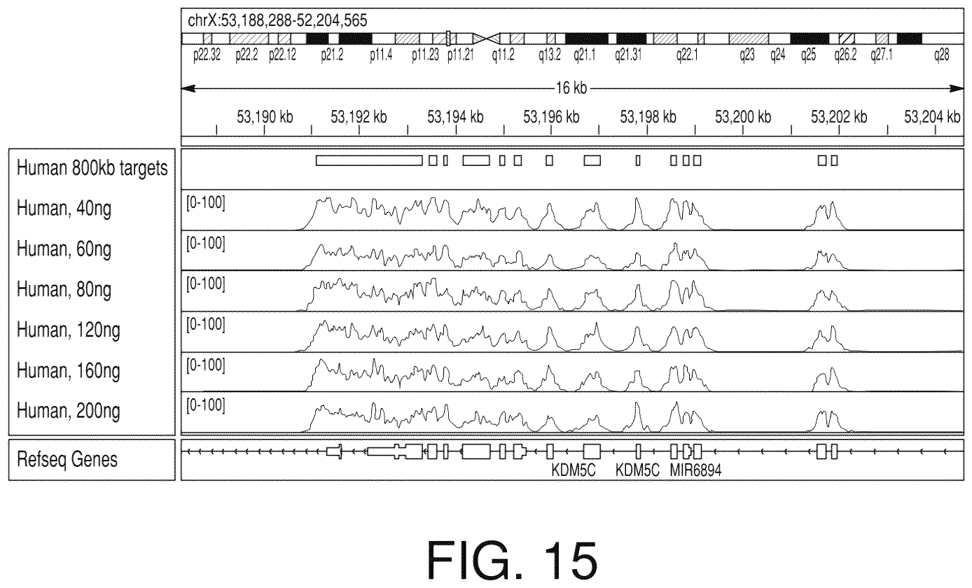

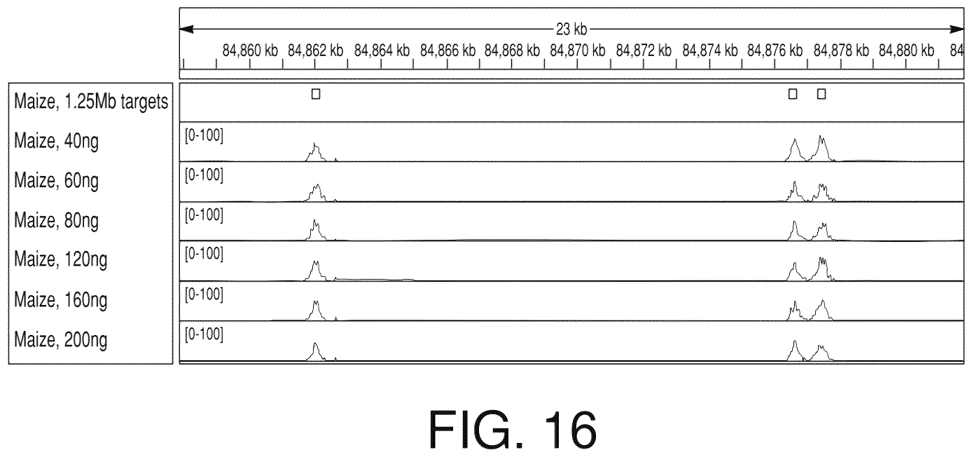

Mean target coverage, uniformity, and genome coverage after target enrichment (TE) were captured for both human samples (800 kb panel) and maize samples (1.25 Mb panel), and read depth downsampled to 150× per library in pool. The duplication rate for human samples was about 15%, while the duplication rate for maize samples was about 32%. As shown in FIG. 13 and FIG. 14, mean target coverage and uniformity after TE were relatively uniform across the range of mass inputs. FIG. 15 demonstrates genome coverage for human KDM5C locus and FIG. 16 displays genome coverage for maize Chr5 locus, both graphs displaying one replicate for each mass input from the twelve-plex.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising:
a first polynucleotide adapter comprising:
a first strand, wherein the first strand comprises a first terminal adapter region, a first non-complementary region, and a first yoke region;
a second strand, wherein the second strand comprises a second terminal adapter region, a second non-complementary region, and a second yoke region;
wherein the first yoke region and the second yoke region are complementary, wherein the first non-complementary region and the second non-complementary region are not complementary,
a second polynucleotide adapter comprising:
a third strand, wherein the third strand comprises a third terminal adapter region, a third non-complementary region, and a third yoke region; and
a fourth strand, wherein the fourth strand comprises a fourth terminal adapter region, a fourth non-complementary region, and a fourth yoke region,
wherein the third yoke region and the fourth yoke region are complementary, and wherein the third non-complementary region and the fourth non-complementary region are not complementary, and
wherein the first polynucleotide adapter and the second polynucleotide adapter are connected via a linker.

2. The composition of claim 1, wherein the linker is attached to the first terminal adapter region and the third terminal adapter region.

3. The composition of claim 1, wherein the linker is attached to the second terminal adapter region and the fourth terminal adapter region.

4. The composition of claim 1, wherein the linker comprises:
an alkene, alkyne, triazine, triazole, oxime, sulfide, amide, and/or cyclooctane; and/or
at least one PEG unit; and/or
at least one Spacer 18 (Sp18).

5. The composition of claim 1, wherein the linker comprises a polynucleotide.

6. The composition of claim 5, wherein the polynucleotide comprises:
at least one cleavable base; and/or
a duplex region; and/or
at least one splint polynucleotide.

7. The composition of claim 6, wherein the polynucleotide comprises at least one splint polynucleotide, wherein the at least one splint polynucleotide is at least partially complementary to a portion of the first polynucleotide adapter or the second polynucleotide adapter.

8. The composition of claim 7, wherein the polynucleotide comprises at least two splint polynucleotides, wherein the at least two splint polynucleotides are at least partially overlapped with each other.

9. The composition of claim 7, wherein the polynucleotide comprises at least two splint polynucleotides, wherein the at least two splint polynucleotides are at least partially overlapped with a portion of the first polynucleotide adapter or the second polynucleotide adapter.

10. The composition of claim 1, wherein one or more of the first polynucleotide adapter and the second polynucleotide adapter comprises at least one barcode.

11. The composition of claim 10, wherein the at least one barcode comprises one or more of a cell index, a sample index, or a unique molecular identifier.

12. (canceled)

13. The composition of claim 1, wherein a sample polynucleotide is attached to the 3′ end of the first strand and the 3′ end or the 5′ end of the second strand.

14. The composition of claim 1, wherein a sample polynucleotide is attached to the 3′ end or the 5′ end of the third strand and the 5′ end of the fourth strand.

15. A method of generating the composition of claim 1, the method comprising:
hybridizing the first strand to the second strand to form a first polynucleotide adapter;
hybridizing the third strand to the fourth strand to form the second polynucleotide adapter; and
contacting the first strand with the third strand, wherein the first strand comprises the linker, wherein the linker comprises a first reactive group and the third strand comprises a second reactive group, and wherein reaction of the first reactive group and the second reactive group generates the composition.

16-18. (canceled)

19. The method of claim 15, wherein the linker comprises at least one splint polynucleotide.

20. The method of claim 19, wherein the at least one splint polynucleotide is at least partially complementary to a portion of the first polynucleotide adapter or the second polynucleotide adapter.

21. The method of claim 19, wherein the linker comprises at least two splint polynucleotides, wherein the at least two splint polynucleotides are at least partially overlapped with each other.

22. The method of claim 15, wherein one or more of the first polynucleotide adapter and the second polynucleotide adapter comprises at least one barcode.

23. The method of claim 15, further comprising attaching a sample polynucleotide to a 3′ end of the first strand and a 3′ end or a 5′ end of the second strand.

24. The method of claim 15, further comprising attaching a sample polynucleotide is attached to the 3′ end or the 5′ end of the third strand and the 5′ end of the fourth strand.


SEQUENCE LISTING (112 sequences)

SEQ ID NO: 1-32 - skipped (32 entries)

SEQ ID NO: 33 - DNA, 20 bp, synthetic construct
  source 1..20: mol_type = other DNA
aatgatacggcgaccaccga

SEQ ID NO: 34 - DNA, 24 bp, synthetic construct
  source 1..24: mol_type = other DNA
caagcagaagacggcatacgagat

SEQ ID NO: 35 - skipped

SEQ ID NO: 36 - DNA, 33 bp, synthetic construct
  source 1..33: mol_type = other DNA
  modified_base 32^33: mod_base = OTHER; note = phosphorothioate bond
acactctttccctacacgacgctcttccgatct

SEQ ID NO: 37 - DNA, 33 bp, synthetic construct
  source 1..33: mol_type = other DNA
  modified_base 32^33: mod_base = OTHER; note = phosphorothioate bond
gatcggaagagcacacgtctgaactccagtcac

SEQ ID NO: 38 - DNA, 33 bp, synthetic construct; identical to SEQ ID NO: 37
  source 1..33: mol_type = other DNA
  modified_base 1^2: mod_base = OTHER; note = phosphorothioate bond

SEQ ID NO: 39 - DNA, 34 bp, synthetic construct
  source 1..34: mol_type = other DNA
  modified_base 1: mod_base = OTHER; note = uracil
  modified_base 33^34: mod_base = OTHER; note = phosphorothioate bond
tacactctttccctacacgacgctcttccgatct

SEQ ID NO: 40 - DNA, 35 bp, synthetic construct
  source 1..35: mol_type = other DNA
  modified_base 1: mod_base = OTHER; note = uracil
  modified_base 2: mod_base = OTHER; note = uracil
  modified_base 34^35: mod_base = OTHER; note = phosphorothioate bond
ttacactctttccctacacgacgctcttccgatct

SEQ ID NO: 41 - DNA, 34 bp, synthetic construct
  source 1..34: mol_type = other DNA
  modified_base 34: mod_base = OTHER; note = uracil
  modified_base 32^33: mod_base = OTHER; note = phosphorothioate bond
gatcggaagagcacacgtctgaactccagtcact

SEQ ID NO: 42 - DNA, 30 bp, synthetic construct
  source 1..30: mol_type = other DNA
tcgtcgcaatggtctcacgagggcacgtac

SEQ ID NO: 43 - DNA, 22 bp, synthetic construct
  source 1..22: mol_type = other DNA
ccaggtatcgtgtaagtagcga

SEQ ID NO: 44 - DNA, 30 bp, synthetic construct
  source 1..30: mol_type = other DNA
gtacgtgccctcgtgagaccattgcgacga

SEQ ID NO: 45 - DNA, 40 bp, synthetic construct
  source 1..40: mol_type = other DNA
tcacagctgtggattggattgccctccgagtgagcgtggc

SEQ ID NO: 46 - DNA, 40 bp, synthetic construct
  source 1..40: mol_type = other DNA
gccacgctcactcggagggcaatccaatccacagctgtga

SEQ ID NO: 47 - DNA, 34 bp, synthetic construct
  source 1..34: mol_type = other DNA
  modified_base 1^2: mod_base = OTHER; note = phosphorothioate bond
gatcggaagagcacacgtctgaactccagtcaca

SEQ ID NO: 48 - DNA, 34 bp, synthetic construct; identical to SEQ ID NO: 41
  source 1..34: mol_type = other DNA
  modified_base 34: mod_base = OTHER; note = uracil
  modified_base 1^2: mod_base = OTHER; note = phosphorothioate bond

SEQ ID NO: 49 - DNA, 37 bp, synthetic construct
  source 1..37: mol_type = other DNA
  modified_base 36^37: mod_base = OTHER; note = phosphorothioate bond
acactctttccctacacgacgctcttccgatctnnnt

SEQ ID NO: 50 - DNA, 37 bp, synthetic construct
  source 1..37: mol_type = other DNA
  modified_base 36^37: mod_base = OTHER; note = phosphorothioate bond
nnnagatcggaagagcacacgtctgaactccagtcac

SEQ ID NO: 51 - DNA, 40 bp, synthetic construct
  source 1..40: mol_type = other DNA
  modified_base 39^40: mod_base = OTHER; note = phosphorothioate bond
acactctttccctacacgacgctcttccgatctgtcgtat

SEQ ID NO: 52 - DNA, 40 bp, synthetic construct
  source 1..40: mol_type = other DNA
  modified_base 39^40: mod_base = OTHER; note = phosphorothioate bond
tacgacagatcggaagagcacacgtctgaactccagtcac

SEQ ID NO: 53 - DNA, 41 bp, synthetic construct
  source 1..41: mol_type = other DNA
  modified_base 40^41: mod_base = OTHER; note = phosphorothioate bond
acactctttccctacacgacgctcttccgatctaacgatgt

SEQ ID NO: 54 - DNA, 41 bp, synthetic construct
  source 1..41: mol_type = other DNA
  modified_base 40^41: mod_base = OTHER; note = phosphorothioate bond
catcgttagatcggaagagcacacgtctgaactccagtcac

SEQ ID NO: 55 - DNA, 20 bp, synthetic construct
  source 1..20: mol_type = other DNA
caaagtgcgattctacgacc

SEQ ID NO: 56 - DNA, 20 bp, synthetic construct
  source 1..20: mol_type = other DNA
ggtcgtagaatcgcactttg

SEQ ID NO: 57 - DNA, 15 bp, synthetic construct
  source 1..15: mol_type = other DNA
tgcgattctacgacc

SEQ ID NO: 58 - DNA, 15 bp, synthetic construct
  source 1..15: mol_type = other DNA
ggtcgtagaatcgca

SEQ ID NO: 59 - DNA, 10 bp, synthetic construct
  source 1..10: mol_type = other DNA
cccggcgacc

SEQ ID NO: 60 - DNA, 10 bp, synthetic construct
  source 1..10: mol_type = other DNA
ggtcgccggg

SEQ ID NO: 61 - DNA, 50 bp, synthetic construct
  source 1..50: mol_type = other DNA
  modified_base 49^50: mod_base = OTHER; note = phosphorothioate bond
tctcacagtgacactctttccctacacgacgctcttccgatctgtcgtat

SEQ ID NO: 62 - DNA, 51 bp, synthetic construct
  source 1..51: mol_type = other DNA
  modified_base 50^51: mod_base = OTHER; note = phosphorothioate bond
tctcacagtgacactctttccctacacgacgctcttccgatctaacgatgt

SEQ ID NO: 63 - DNA, 60 bp, synthetic construct
  source 1..60: mol_type = other DNA
  modified_base 59^60: mod_base = OTHER; note = phosphorothioate bond
tctcacagtgaactacgcctacactctttccctacacgacgctcttccgatctgtcgtat

SEQ ID NO: 64 - DNA, 61 bp, synthetic construct
  source 1..61: mol_type = other DNA
  modified_base 60^61: mod_base = OTHER; note = phosphorothioate bond
tctcacagtgaactacgcctacactctttccctacacgacgctcttccgatctaacgatgt

SEQ ID NO: 65 - DNA, 30 bp, synthetic construct
  source 1..30: mol_type = other DNA
caaagtgcgattctacgaccaactacgcct

SEQ ID NO: 66 - DNA, 30 bp, synthetic construct
  source 1..30: mol_type = other DNA
ggtcgtagaatcgcactttgaactacgcct

SEQ ID NO: 67 - DNA, 80 bp, synthetic construct
  source 1..80: mol_type = other DNA
  modified_base 79^80: mod_base = OTHER; note = phosphorothioate bond
tctcacagtgaactacgcctaggtttatggaatacagctaacactctttccctacacgacgctcttccgatctgtcgtat

SEQ ID NO: 68 - DNA, 80 bp, synthetic construct
  source 1..80: mol_type = other DNA
  modified_base 79^80: mod_base = OTHER; note = phosphorothioate bond
tctcacagtgaactacgcctaggtttatggaatacagctacactctttccctacacgacgctcttccgatctaacgatgt

SEQ ID NO: 69-80 - skipped (12 entries)

SEQ ID NO: 81 - DNA, 37 bp, synthetic construct; identical to SEQ ID NO: 49
  source 1..37: mol_type = other DNA
  modified_base 36^37: mod_base = OTHER; note = phosphorothioate bond
  misc_difference 34^35: replace = n

SEQ ID NO: 82 - DNA, 37 bp, synthetic construct; identical to SEQ ID NO: 49
  source 1..37: mol_type = other DNA
  modified_base 36^37: mod_base = OTHER; note = phosphorothioate bond
  misc_difference 34^35: replace = nn

SEQ ID NO: 83 - DNA, 37 bp, synthetic construct; identical to SEQ ID NO: 49
  source 1..37: mol_type = other DNA
  modified_base 36^37: mod_base = OTHER; note = phosphorothioate bond
  misc_difference 34^35: replace = nnn

SEQ ID NO: 84 - DNA, 37 bp, synthetic construct; identical to SEQ ID NO: 49
  source 1..37: mol_type = other DNA
  modified_base 36^37: mod_base = OTHER; note = phosphorothioate bond
  misc_difference 34^35: replace = nnnn

SEQ ID NO: 85 - DNA, 37 bp, synthetic construct; identical to SEQ ID NO: 50
  source 1..37: mol_type = other DNA
  misc_difference 34^35: replace = n
  modified_base 36^37: mod_base = OTHER; note = phosphorothioate bond

SEQ ID NO: 86 - DNA, 37 bp, synthetic construct; identical to SEQ ID NO: 50
  source 1..37: mol_type = other DNA
  misc_difference 34^35: replace = nn
  modified_base 36^37: mod_base = OTHER; note = phosphorothioate bond

SEQ ID NO: 87 - DNA, 37 bp, synthetic construct; identical to SEQ ID NO: 50
  source 1..37: mol_type = other DNA
  misc_difference 34^35: replace = nnn
  modified_base 36^37: mod_base = OTHER; note = phosphorothioate bond

SEQ ID NO: 88 - DNA, 40 bp, synthetic construct
  source 1..40: mol_type = other DNA
  modified_base 39^40: mod_base = OTHER; note = phosphorothioate bond
acactctttccctacacgacgctcttccgatctacatcat

SEQ ID NO: 89 - DNA, 40 bp, synthetic construct
  source 1..40: mol_type = other DNA
  modified_base 39^40: mod_base = OTHER; note = phosphorothioate bond
acactctttccctacacgacgctcttccgatctacgactt

SEQ ID NO: 90 - DNA, 41 bp, synthetic construct
  source 1..41: mol_type = other DNA
  modified_base 40^41: mod_base = OTHER; note = phosphorothioate bond
acactctttccctacacgacgctcttccgatctaggataat

SEQ ID NO: 91 - DNA, 41 bp, synthetic construct
  source 1..41: mol_type = other DNA
  modified_base 40^41: mod_base = OTHER; note = phosphorothioate bond
acactctttccctacacgacgctcttccgatctatggtgat

SEQ ID NO: 92 - DNA, 40 bp, synthetic construct
  source 1..40: mol_type = other DNA
  modified_base 39^40: mod_base = OTHER; note = phosphorothioate bond
acactctttccctacacgacgctcttccgatctcaactgt

SEQ ID NO: 93 - DNA, 40 bp, synthetic construct
  source 1..40: mol_type = other DNA
  modified_base 39^40: mod_base = OTHER; note = phosphorothioate bond
acactctttccctacacgacgctcttccgatctccgatgt

SEQ ID NO: 94 - DNA, 40 bp, synthetic construct
  source 1..40: mol_type = other DNA
  modified_base 39^40: mod_base = OTHER; note = phosphorothioate bond
acactctttccctacacgacgctcttccgatctctggttt

SEQ ID NO: 95 - DNA, 40 bp, synthetic construct
  source 1..40: mol_type = other DNA
  modified_base 39^40: mod_base = OTHER; note = phosphorothioate bond
acactctttccctacacgacgctcttccgatctgaagcat

SEQ ID NO: 96 - DNA, 41 bp, synthetic construct
  source 1..41: mol_type = other DNA
  modified_base 40^41: mod_base = OTHER; note = phosphorothioate bond
acactctttccctacacgacgctcttccgatctgtagccat

SEQ ID NO: 97 - DNA, 41 bp, synthetic construct
  source 1..41: mol_type = other DNA
  modified_base 40^41: mod_base = OTHER; note = phosphorothioate bond
acactctttccctacacgacgctcttccgatcttcctgact

SEQ ID NO: 98 - DNA, 40 bp, synthetic construct
  source 1..40: mol_type = other DNA
  modified_base 39^40: mod_base = OTHER; note = phosphorothioate bond
acactctttccctacacgacgctcttccgatcttcttggt

SEQ ID NO: 99 - DNA, 40 bp, synthetic construct
  source 1..40: mol_type = other DNA
  modified_base 39^40: mod_base = OTHER; note = phosphorothioate bond
acactctttccctacacgacgctcttccgatcttggcttt

SEQ ID NO: 100 - DNA, 40 bp, synthetic construct
  source 1..40: mol_type = other DNA
  modified_base 39^40: mod_base = OTHER; note = phosphorothioate bond
tgatgtagatcggaagagcacacgtctgaactccagtcac

SEQ ID NO: 101 - DNA, 40 bp, synthetic construct
  source 1..40: mol_type = other DNA
  modified_base 39^40: mod_base = OTHER; note = phosphorothioate bond
agtcgtagatcggaagagcacacgtctgaactccagtcac

SEQ ID NO: 102 - DNA, 41 bp, synthetic construct
  source 1..41: mol_type = other DNA
  modified_base 40^41: mod_base = OTHER; note = phosphorothioate bond
ttatcctagatcggaagagcacacgtctgaactccagtcac

SEQ ID NO: 103 - DNA, 41 bp, synthetic construct
  source 1..41: mol_type = other DNA
  modified_base 40^41: mod_base = OTHER; note = phosphorothioate bond
tcaccatagatcggaagagcacacgtctgaactccagtcac

SEQ ID NO: 104 - DNA, 41 bp, synthetic construct; identical to SEQ ID NO: 103
  source 1..41: mol_type = other DNA
  modified_base 40^41: mod_base = OTHER; note = phosphorothioate bond

SEQ ID NO: 105 - DNA, 40 bp, synthetic construct
  source 1..40: mol_type = other DNA
  modified_base 39^40: mod_base = OTHER; note = phosphorothioate bond
cagttgagatcggaagagcacacgtctgaactccagtcac

SEQ ID NO: 106 - DNA, 40 bp, synthetic construct
  source 1..40: mol_type = other DNA
  modified_base 39^40: mod_base = OTHER; note = phosphorothioate bond
catcggagatcggaagagcacacgtctgaactccagtcac

SEQ ID NO: 107 - DNA, 40 bp, synthetic construct
  source 1..40: mol_type = other DNA
  modified_base 39^40: mod_base = OTHER; note = phosphorothioate bond
aaccagagatcggaagagcacacgtctgaactccagtcac

SEQ ID NO: 108 - DNA, 40 bp, synthetic construct
  source 1..40: mol_type = other DNA
  modified_base 39^40: mod_base = OTHER; note = phosphorothioate bond
tgcttcagatcggaagagcacacgtctgaactccagtcac

SEQ ID NO: 109 - DNA, 41 bp, synthetic construct
  source 1..41: mol_type = other DNA
  modified_base 40^41: mod_base = OTHER; note = phosphorothioate bond
tggctacagatcggaagagcacacgtctgaactccagtcac

SEQ ID NO: 110 - DNA, 41 bp, synthetic construct
  source 1..41: mol_type = other DNA
  modified_base 40^41: mod_base = OTHER; note = phosphorothioate bond
gtcaggaagatcggaagagcacacgtctgaactccagtcac

SEQ ID NO: 111 - DNA, 40 bp, synthetic construct
  source 1..40: mol_type = other DNA
  modified_base 39^40: mod_base = OTHER; note = phosphorothioate bond
ccaagaagatcggaagagcacacgtctgaactccagtcac

SEQ ID NO: 112 - DNA, 40 bp, synthetic construct
  source 1..40: mol_type = other DNA
  modified_base 39^40: mod_base = OTHER; note = phosphorothioate bond
aagccaagatcggaagagcacacgtctgaactccagtcac